(19) United States
(12) Patent Application Publication
(10) Pub. No.: US 2026/0240913 A1
(43) Pub. Date: Aug. 20, 2026

(54) METHODS FOR PRODUCING STABLE HUMAN CHONDROCTYES AND THEIR USE FOR PROMOTING CARTILLAGE GROWTH AND REPAIR

(71) Applicants: Cambridge Enterprise Limited, Cambridge (GB); The United States of America, as represented by the Secretary, Department of Health and Human Servic, Bethesda, MD (US)

(72) Inventors: Pamela Gehron Robey, Bethesda, MD (US); Stephen Joseph Gathesda, Bethesda, MD (US); Andrew McCaskie, Cambridge (GB)

(73) Assignees: Cambridge Enterprise Limited, Cambridge (GB); The United States of America, as represented by the Secretary, Department of Health and Human Servic, Bethesda, MD (US)

(21) Appl. No.: 19/165,187

(22) PCT Filed: Mar. 15, 2024
(86) PCT No.: PCT/US2024/020102

§ 371 Date: Sep. 15, 2025

Related U.S. Application Data
(60) Provisional application No. 63/490,759, filed on Mar. 16, 2023.

(51) Int. Cl. A61K 35/32; A61K 47/64; C12N 5/077

(52) U.S. Cl. CPC A61K 35/32; A61K 47/6435; C12N 5/0655; C12N 2501/155

(57) ABSTRACT

Isolated non-natural human chondrocytes are disclosed that have decreased expression of Type X Collagen (COL10A1) as compared with hypertrophic chondrocytes. The non-natural human chondrocytes are derived from an induced pluripotent stem cell (iPSC). In some aspects, the non-natural human chondrocytes do not undergo hypertrophy for at least 42 days in vitro. In more aspects, the non-natural human chondrocytes do not undergo hypertrophy for at least 5 months when transplanted in vivo. In some aspects, disclosed are methods of producing these non-natural human chondrocytes. In further aspects, disclosed am methods of using these non-natural human chondrocytes, such as for promoting cartilage growth and/or repair.

### CROSS REFERENCE TO RELATED APPLICATIONS

This claims the benefit of U.S. Provisional Application No. 63/490,759, filed Mar. 16, 2023, which is incorporated by reference herein.

### STATEMENT OF GOVERNMENT SUPPORT

This invention was made with Government support under project number Z01 #: Z1A-DE000380 by the National Institutes of Health, National Institute of Dental and Craniofacial Research. The United States Government has certain rights in the invention.

### SEQUENCE LISTING

The Sequence Listing is submitted as an XML file in the form of the file named “Sequence.xml” (74,829 bytes), which was created on Mar. 13, 2024, which is incorporated by reference herein.

### FIELD OF THE DISCLOSURE

This relates to the field of the differentiation of human induced pluripotent stem cells (hiPSCs), specifically to the production of non-natural chondrocytes and their use for treatment.

### BACKGROUND

Osteoarthritis (OA), a disease characterized by the permanent loss of articular cartilage that lines joint surfaces, is among the most common disabling diseases worldwide, affecting 40% of people over the age of 70. Although surgical joint replacements provide pain relief and restore quality of life, this option is typically indicated for more advanced stages of OA. Joint replacement in younger patients is associated with an increased risk of revision surgery and therefore OA in this group, particularly the earlier stages of disease, represents an area of unmet clinical need.

Since articular cartilage is avascular and damaged cartilage in OA has limited ability to repair, cell therapy has the potential to effectively treat OA, yet current strategies are largely ineffective. For instance, microfracture—a technique by which small holes are drilled in the subchondral bone to recruit resident BMSCs/SSCs to the affected area—is limited by the overproduction of fibrocartilage, which is dense in Type I collagen and less suited to reduce friction with joint movement compared with normal hyaline cartilage. Similarly, direct implantation of hBMSCs/SSCs into arthritic joints in several human trials had limited benefits in restoring the architecture of hyaline cartilage and relieving pain. Autologous chondrocyte implantation—a process by which healthy chondrocytes from less weight-bearing areas are removed, expanded ex vivo, and transplanted into the affected joint—is limited by its ex vivo expansion methods, whereby chondrocytes dedifferentiate and begin to acquire a fibroblastic phenotype. A similar procedure, osteochondral graft surgery, whereby the subchondral bone and overlying cartilage is removed and transplanted into a more severe articular damage or injury, is limited by donor site morbidity and incomplete coverage of the damaged area (referred to as mosaicplasty), leading to suboptimal healing. Therefore, current cell therapy is limited by an inadequate development and stability of articular chondrocytes that maintain hyaline cartilage. A need remains for a method that produces chondrocytes that are stable when transplanted in vivo.

### SUMMARY OF THE DISCLOSURE

Isolated non-natural human chondrocytes are disclosed that have decreased expression of Type X Collagen (COL10A1) as compared with hypertrophic chondrocytes. The non-natural human chondrocytes are derived from an induced pluripotent stem cell (iPSC). In some aspects, the non-natural human chondrocytes do not undergo hypertrophy for at least 42 days in vitro. In more aspects, the non-natural human chondrocytes do not undergo hypertrophy for at least 5 months when transplanted in vivo.

In some aspects, disclosed are methods of producing these non-natural human chondrocytes. In further aspects, disclosed are methods of using these non-natural human chondrocytes, such as for promoting cartilage growth and/or repair. These methods include administering locally to a site in a subject in need thereof, a therapeutically effective amount of a conjugate comprising the non-natural human chondrocytes and a solid carrier, thereby producing stable cartilage locally at the site in the subject. In some aspects, the subject has, or is at risk of having, cartilage damage or degradation.

The foregoing and other features and advantages of the disclosure will become more apparent from the following detailed description of several aspects which proceeds with reference to the accompanying figures.

### BRIEF DESCRIPTION OF THE FIGURES

FIGS. 1A-1D. HyA-FMBs promote early expression of extracellular matrix genes. (A) Schematic of hBMSC/SSC culture±HyA-FMBs and digestion of organoids at days 1, 3, 5, and 10 for scRNA-seq. (B) Toluidine Blue staining of control and HyA-FMB organoids during chondrogenic differentiation. Scale bars, 300 μm. (C) Expression of collagen genes ordered by pseudotime from days 1, 3, 5, and 10 of chondrogenic differentiation in control and HyA-FMB organoids. Each dot represents a single cell. (D) Expression of non-collagenous genes ordered by pseudotime from days 1, 3, 5, and 10 of chondrogenic differentiation in control and HyA-FMB organoids. Each dot represents a single cell.

FIGS. 2A-2L. HyA-FMBs suppress BMP signaling early in chondrogenic differentiation. (A-B) Gene enrichment analysis of KEGG signal transduction pathways from differentially expressed genes among control and HyA-FMB organoids (log fc>0.25) at day 1 (A) and day 3 (B). TGFβ signaling pathway was consistently enriched in control organoids. (C) Violin plots depicting gene expression from control and HyA-FMB organoids after 1 day of chondrogenic differentiation (Donor #1: 7,541 control and 9,979 HyA-FMB cells; Donor #2: 9,169 control and 8,024 HyA-FMB cells). Asterisks indicate trends across two donors. Genes associated with TGFβ signaling, BMP signaling, and chondro-osteogenesis are shown. (D) Feature plots highlighting expression of selected genes from control and HyA-FMB organoids after 1 day of chondrogenic differentiation. (E) Violin plots depicting gene expression from control and HyA-FMB organoids after 3 days of chondrogenic differentiation (Donor #1: 3,832 control and 7,895 HyA-FMB cells; Donor #2: 5,765 control and 4,992 HyA-FMB cells). Asterisks indicate trends across two donors. Genes associated with TGFβ signaling, BMP signaling, and chondro-osteogenesis are shown. (F) Feature plots highlighting expression of selected genes from control and HyA-FMB organoids after 3 days of chondrogenic differentiation. Dashed lines indicate early partitioning of IGFBP5⁺ cells in HyA-FMB organoids. (G, I, K) Immunofluorescence of non-collagenous proteins in day 3 control and HyA-FMB organoids. High magnification insets shown to the right of their corresponding images. Nuclei counterstained with DAPI. Scale bars, 200 μm. (H, J, L) Area quantification of non-collagenous protein expression in day 3 control and HyA-FMB organoids. Each dot represents a biological replicate. Data are mean±SEM; *p<0.05, unpaired two-tailed t test.

FIGS. 3A-3H. HyA-FMBs restore and activate BMP signaling in MGP/IGFBP5-enriched chondrogenic cells. (A) UMAP representation of combined control and HyA-FMB datasets after 5 days of chondrogenic differentiation with annotated clusters. A total of 6,110 control and 6,749 HyA-FMB cells were included following quality control measures. (B) Violin plots depicting differential gene expression from combined control and HyA-FMB datasets after 5 days of chondrogenic differentiation. (C) Bar chart depicting proportion of cell clusters in control and HyA-FMB datasets after 5 days of chondrogenic differentiation. (D) Violin plots depicting gene expression split by experimental condition from combined datasets after 5 days of chondrogenic differentiation. The boxes highlight similarities in MGP^{hi}/IGFBP5^{hi} cluster, and differences in SPP1⁺/IBSP⁺ cluster. Asterisks indicate trends in genes associated with TGFβ signaling, BMP signaling, and chondro-osteogenesis. (E) UMAP representation of combined control and HyA-FMB datasets after 10 days of chondrogenic differentiation with annotated clusters. A total of 12,988 control and 10,582 HyA-FMB cells were included following quality control measures. (F) Violin plots depicting differential gene expression from combined control and HyA-FMB datasets after 10 days of chondrogenic differentiation. (G) Bar chart depicting proportion of cell clusters in control and HyA-FMB datasets after 10 days of chondrogenic differentiation. (H) Violin plots depicting gene expression split by experimental condition from combined datasets after 10 days of chondrogenic differentiation. The box highlights differences in MGP^{hi}/IGFBP5^{hi} cluster. Asterisks indicate trends in genes associated with TGFβ signaling, BMP signaling, and chondro-osteogenesis.

FIGS. 4A-4H. Rat chondral transplantation of hBMSC/SSC/HyA-FMB constructs yields suboptimal chondrogenesis. (A) Schematic of ectopic transplant of hBMSCs/SSCs attached to HyA-FMBs, followed by digestion and scRNA-seq of transplanted tissue 8 weeks-post-transplant. (B) Toluidine Blue (left) and H&E (right) staining of ectopic transplant of hBMSCs/SSCs attached to HyA-FMBs at 8 weeks-post-transplant. (C). Immunostaining of transplants for pSMAD5, indicative of BMP signaling in chondrocytes at 8 weeks-post transplant. Scale bars, 500 μm. (D) Dot plot demonstrating global gene expression of integrated in vitro HyA-FMB datasets (Day 0: 7425 cells, Day 1: 9979 cells, Day 3: 7898 cells, Day 5: 6028 cells, Day 10: 10,582 cells) and in vivo transplant of hBMSCs/SSCs attached to HyA-FMBs analyzed 8 weeks-post-transplant (29,220 cells). Genes associated with BMP signaling and chondro-osteogenesis are shown. (E) Schematic of chondral transplants: passage 2 hBMSCs/SSCs were detached and incubated with HyA-FMBs for 2 hours, followed by transplantation of cell mixture into a 2-mm defect at the rat femoral trochlear groove. (F) Toluidine Blue staining of defect areas (dashed lines) from chondral transplants (without cells—left; with cells—right) at 1, 2, and 4 months-post transplant in SRG rats. Scale bars, 500 μm. (G) Schematic of organoid chondral transplants: hBMSCs/SSCs were cultured with HyA-FMBs in chondrogenic medium for 10 days, followed by transplantation of HyA-FMB organoids in a 2-mm defect at the rat femoral trochlear groove. (H) Toluidine Blue staining of defect areas (dashed lines) from sham operated (left) and organoid chondral transplants (right) at 1, 2, and 4 months-post transplant in SRG rats. Scale bars, 500 μm.

FIGS. 5A-5E. BMP activation in SOX9⁺ purified pre-chondrogenic cells promotes stable chondrogenesis in vitro. (A) Schematic of sclerotome and chondrogenic differentiation strategy from hiPSCs: pathway activators, inhibitors, and recombinant growth factors are shown; these were added across 6 days of adherent culture, after which chondrogenic medium was added supplemented with TGFβ1, BMP2, and GDF5. (B) Area quantification from confocal analyses of tissues derived from three chondrogenic differentiation strategies. N=2-4 replicates. Data are mean±SEM. (C) SOX9-mCherry culture area and fluorescence intensity of SOX9-mCherry hiPSCs across 16 days of differentiation on monolayer cultures (third strategy), measured by Incucyte analysis software. Sum of three independent experiments. (D) Representative brightfield and mCherry fluorescence images depicting cell morphology across differentiation, and appearance of SOX9-mCherry^{bright} chondrospheroids by 16 days of adherent culture. (E) Toluidine Blue staining and immunofluorescence analyses of chondrospheroids across time. Nuclei counterstained with DAPI. Scale bars, 300 μm.

FIGS. 6A-6H. Chondrospheroid transcriptomes reveal a fetal-like chondrogenic identity. (A) PCA of hiPSC, sclerotome, and chondrospheroid datasets. Each dot represents a technical replicate. (B-E) GO analysis representing top 5 pathways from sclerotome (B), day 14 (C), day 28 (D), and day 42 chondrospheroids (E). (F) Heatmap depicting differential gene expression: top 100 differentially expressed genes from each dataset were assessed against GO pathways listed in B-E, reducing the list to 103 relevant genes. (G) PCA of hiPSC, sclerotome, and chondrospheroid datasets, which were batch-corrected and normalized to previously published human 5-6-week-old embryonic, 17-week-old fetal, adolescent, and adult primary chondrocytes. (H) Hierarchical clustering analysis of sclerotome, chondrospheroid, and primary human chondrocyte populations.

FIGS. 7A-7G. Chondral transplantation of hiPSC/HyA-FMB constructs yields stable chondrogenesis. (A) Schematic of chondral transplantation of day 35 chondrospheroid cells attached to HyA-FMBs, followed by histology and immunofluorescence analysis in NSG mice (B-F) and SRG rats (G). (B) Toluidine Blue staining of defect areas from transplanted with HyA-FMBS only (top left), and day 35 chondrospheroid cells attached to HyA-FMBs at 1-5 months-post-transplant in NSG mice (pictures in dashed line box). High magnification insets shown to the right of their corresponding images. White arrows highlight the formation of bone in control HyA-FMB only transplants at 1 month. Scale bars, 500 μm. (C-F) Immunofluorescence analyses of femoral defects from NSG mice 1 month-post-transplant. High magnification insets depicting transplanted cells, surface articular cartilage, and growth plate cartilage shown to the right of their corresponding images. Nuclei counterstained with DAPI. Scale bars, 200 μm. (G) Toluidine Blue staining of defect areas from transplanted day 35 chondrospheroid cells attached to HyA-FMBs at 2 and 5 months-post-transplant in SRG rats (right). Left images depict control transplants, including HyA-FMBs only and hBMSCs/SSCs attached to HyA-FMBs at 2 months. High magnification insets shown to the right of their corresponding images. Scale bars, 500 μm.

FIGS. 8A-8D. Related to FIG. 1. TGFβ1 required for proper chondrogenic differentiation in control and HyA-FMB organoids. (A-B) Toluidine Blue staining of control and HyA-FMB organoids during chondrogenic differentiation in the absence of TGFβ1 (A) or supplemented with TGFβ1 (B). Scale bars, 300 μm. (C) Expression of primitive BMSC/SSC genes ordered by pseudotime from days 1, 3, 5, and 10 of chondrogenic differentiation in control and HyA-FMB organoids. Each dot represents a single cell. (D) Expression of chondrogenic and osteogenic transcription factors ordered by pseudotime from days 1, 3, 5, and 10 of chondrogenic differentiation in control and HyA-FMB organoids. Each dot represents a single cell.

FIGS. 9A-9B. Related to FIG. 2. HyA-FMBs suppress BMP signaling early in chondrogenic differentiation. (A-B) Gene enrichment analysis of KEGG TGFβ signaling pathway from differentially expressed genes (log fc>0.25) in HyA-FMB organoids compared with controls at day 1 (A) and day 3 (B) of chondrogenic differentiation.

FIGS. 10A-10P. Related to FIG. 3. Chronic suppression of BMP-ID signaling promotes osteogenic gene expression. (A) Schematic depicting treatment of hBMSC/SSC organoids with AGX51 (pan-ID inhibitor) or vehicle every 2-3 days, followed by digestion of organoids at days 3, 7, 14, 21, 28 of chondrogenic differentiation for qRT-PCR analysis of chondro-osteogenic genes. (B-H) qRT-PCR analysis of chondro-osteogenic genes from hBMSC/SSC organoids treated with AGX51 or vehicle across 28 days of chondrogenic differentiation. Each shape represents a biological replicate. Dotted line represents gene expression of day 21 osteogenic cultures derived from hBMSCs/SSCs (positive osteogenic control). Data are mean±SEM; *p<0.05, **p<0.01, unpaired two-tailed t test. (I) Schematic depicting formation of organoids derived from hBMSCs/SSCs transduced with lentiviral vectors containing shRNA against ID1 (siID1) or control shRNA (siGL), followed by digestion of organoids at day 28 of chondrogenic differentiation for qRT-PCR analysis of chondro-osteogenic genes. (J-P) qRT-PCR analysis of chondro-osteogenic genes from day 28 organoids derived from hBMSCs/SSCs transduced with siID1 and control (siGL) vectors, with positive osteogenic control. Each dot represents a technical replicate. Data are mean±SEM; *p<0.05, **p<0.01, unpaired two-tailed t test.

FIGS. 11A-11E. Related to FIG. 4. Rat chondral transplantation of hBMSC/SSC/HyA-FMB constructs yields suboptimal chondrogenesis. (A) Toluidine Blue staining of defect areas from HyA-FMB chondral transplants at 1 week- and 1 month-post transplant in SRG rats, showing dissolution of HyA-FMBs over time. Scale bars, 500 μm. (B) Immunofluorescence analysis of human VIMENTIN to confirm human origin of transplanted hBMSCs/SSCs attached to HyA-FMBs at 2 months-post-transplant. Dashed line indicates boundary separating transplanted cells from rat bone marrow (BM). Nuclei counterstained with DAPI. Scale bars, 500 μm. (C) H&E staining of defect areas (dashed lines) from chondral transplants at 1, 2, and 4 months-post transplant in SRG rats. Scale bars, 500 μm. (D) H&E staining of defect areas (dashed lines) from human BMSC organoid chondral transplants at 1, 2, and 4 months-post transplant in SRG rats. Scale bars, 500 μm. (E) Second harmonic generation imaging of defect area following chondral and organoid chondral transplants (middle and right images), compared with an unoperated control (left image). Scale bars, 500 μm.

FIGS. 12A-12D. Related to FIG. 5. Efficient anterior primitive streak, paraxial mesoderm, and sclerotome Induction In hiPSCs. (A) Schematic of sclerotome differentiation strategy from hiPSCs: pathway activators, inhibitors, and recombinant growth factors were added across 6 days of adherent culture. (B) Heatmap depicting mRNA expression of hiPSC differentiation to anterior primitive streak (APS), paraxial mesoderm (PM), early somite/somitomere (ES), and sclerotome (SCL) from qRT-PCR experiments. Lines are shown that indicate primitive streak markers, paraxial mesoderm markers, somite/sclerotome markers, and lateral and cardiac mesoderm markers. N=3 technical replicates. (C) Schematic of anterior primitive streak differentiation strategy of MIXL1-GFP hiPSCs: pathway activators and inhibitors and recombinant growth factors were added across 24 hours of adherent culture, followed by FACS analysis. (D) Representative FACS plot (left) and quantification (right) of MIXL1-GFP expression in MIXL1-GFP hiPSCs differentiated to anterior primitive streak for 24 hours. Each shape represents a biological replicate. Data are mean±SEM; ***p<0.001, unpaired two-tailed t test.

FIGS. 13A-13B. Related to FIG. 5. Pellet cultures from all sclerotome cells yield Inefficient chondrogenesis and hypertrophy. (A) Toluidine Blue staining and immunofluorescence analyses of pellet cultures constructed from all sclerotome cells, supplemented with TGFβ1 across time (first method). Nuclei counterstained with DAPI. Scale bars, 300 μm. (B) Toluidine Blue staining and immunofluorescence analyses of pellet cultures constructed from all sclerotome cells, supplemented with TGFβ1, GDF-5, and BMP-2 across time (second method). Nuclei counterstained with DAPI. Scale bars, 300 μm.

FIGS. 14A-14D. Related to FIG. 5. hiPSC-derived limb mesoderm cells form aggregates with low SOX9 expression and chondrogenic potential. (A) Schematic of limb mesoderm and chondrogenic differentiation strategy from hiPSCs: pathway activators, inhibitors, and recombinant growth factors were added across 4 days of adherent culture, after which two chondrogenic differentiation strategies were tested. (B) Heatmap depicting mRNA expression of hiPSC differentiation to mid primitive streak (MPS), lateral plate mesoderm (LPM), and limb mesoderm (LM) from qRT-PCR experiments. Primitive streak markers, lateral plate mesoderm markers, limb mesoderm markers, paraxial mesoderm and sclerotome markers are shown. N=3 technical replicates. (C) Incucyte images depicting SOX9-mCherry expression in SOX9-mCherry hiPSC-derived limb mesoderm (left) and sclerotome (right) monolayer cultures after 8 days of treatment with TGFβ, BMP-2, and GDF-5 in chondrogenic medium. (D) Toluidine Blue staining of pellet cultures and aggregates from hiPSC-derived limb mesoderm cells supplemented with TGFβ1, GDF-5, and BMP-2 in chondrogenic medium. Scale bars, 300 μm.

FIGS. 15A-15C. Related to FIG. 6. Chondrospheroid transcriptomes reveal a fetal-Ike chondrogenic identity. (A) Hierarchical clustering analysis of hiPSC, sclerotome, and chondrospheroid datasets. (B) Heatmap depicting genes of interest from hiPSC, sclerotome, and chondrospheroid datasets. (C) Smear Plots of hiPSC, sclerotome, and chondrospheroid datasets, which were batch-corrected and normalized to previously published human 5-6-week-old embryonic, 17-week-old fetal, adolescent, and adult primary chondrocytes. Each plot depicts comparison between two groups of interest with number of differentially expressed genes shown in red (log FC>2, log FC>−2). Each dot represents one gene.

FIGS. 16A-16H. Related to FIG. 7. HyA-FMBs promote stable chondrogenesis in subcutaneous chondrospheroid cell transplants. (A) Schematic of hiPSC differentiation to sclerotome and chondrospheroids, which were transplanted at day 35 using three methods. (B-C) H&E (B) and Toluidine Blue (C) staining of subcutaneous transplants of undigested day 35 chondrospheroids at 1 and 2 months-post-transplant in NSG mice. High magnification insets shown below their corresponding images. (D-E) H&E (D) and Toluidine Blue (E) staining of subcutaneous transplants of digested day 35 chondrospheroid cells embedded in Matrigel at 1 and 2 months-post-transplant in NSG mice. High magnification insets shown below their corresponding images. (F-G) H&E (F) and Toluidine Blue (G) staining of subcutaneous transplants of digested day 35 chondrospheroid cells attached to HyA-FMBs at 1 and 2 months-post-transplant in NSG mice. High magnification insets shown below their corresponding images. (H) Confocal images of pSMAD5 with nuclei counterstained with DAPI in transplants of cells derived from chondrospheroids in either MATRIGEL™ or with HyA-FMBs. High magnification image shown to the right outlined by dashed lines. Scale bars, 300 μm. Quantitation of pSMAD5 shows that BMP signaling was largely absent in the MATRIGEL™ transplants, but robust in HyA-FMB transplants.

FIGS. 17A-17C. Related to FIG. 7. Chondral transplantation of hiPSC/HyA-FMB constructs yields stable chondrogenesis. (A) H&E staining of defect areas from transplanted HyA FMBs only (upper left) and from transplanted day 35 chondrospheroid cells attached to HyA-FMBs at 1-5 months-post-transplant in NSG mice (in dashed box). High magnification insets shown to the right of their corresponding images. Arrows depicting the formation of bone in control HyA FMB only transplants at 1 month (upper left). (B) Immunofluorescence analysis of human VIMENTIN to confirm human origin of transplanted day 35 chondrospheroid cells attached to HyA-FMBs at 1 month-post-transplant. Dashed line indicates boundary separating transplanted cells from mouse bone marrow (BM). Nuclei counterstained with DAPI. (C) H&E staining of defect areas from transplanted day 35 chondrospheroid cells attached to HyA-FMBs at 2 months-post-transplant in SRG rats (right). Left images depict control transplants, including HyA-FMBs only and hBMSCs/SSCs attached to HyA-FMBs at 2 months. High magnification insets shown to the right of their corresponding images.

### SEQUENCES

The nucleic and amino acid sequences listed herein are shown using standard letter abbreviations for nucleotide bases and amino acids. Only one strand of each nucleic acid sequence is shown, but the complementary strand is understood as included by any reference to the displayed strand.

SEQ ID NOs: 1-70 are oligodeoxynucleotide sequences used for polymerase chain reaction (PCR).

SEQ ID NOs: 71-80 are oligodeoxynucleotide sequences used in CRISR/Cas9 engineering.

SEQ ID NO: 81-84 are the nucleic acid sequences of PCR primers.

### DETAILED DESCRIPTION OF SEVERAL ASPECTS

Differentiated chondrocytes from human bone marrow stromal cells including skeletal stem cells (Bone marrow stromal cells (BMSCs)/skeletal stem cells (SSCs)), and from human induced pluripotent stem cells (hiPSCs) have the potential to permanently restore damaged cartilage in arthritic joints, yet chondrocyte hypertrophy is a major barrier for translational therapy. With chondrogenic differentiation, BMSCs/SSCs undergo hypertrophy in vitro and mineralization in vivo, leading to inferior fibrocartilage and bone formation. However, BMSCs/SSCs attached to a fibrin microbead scaffold coated with hyaluronic acid (HyA-FMBs) produce hyaline-like cartilage for up to 28 weeks in vivo. It is disclosed herein, the signaling pathways that govern the development of hypertrophic-resistant chondrocytes using conjugates on an exemplary solid carrier, the HyA-FMB model system, and the differentiation of hiPSCs to stable chondrocyte-like cells using this mechanistic knowledge.

After one day of chondrogenic differentiation in vitro, BMSCs/SSCs attached to HyA-FMBs exhibited higher expression of extracellular matrix proteins—including Insulin-like Growth Factor Binding Protein-5 (IGFBP5) and Matrix Gla Protein (MGP)—and decreased Bone Morphogenic Protein (BMP) signaling, evidenced by pathway analysis. However, BMP signaling was restored by day 5, and increased by day 10, in a chondrogenic subpopulation enriched for IGFBP5 and MGP, accompanied by diminished expression of COL10A1, ALPL, IBSP, and SPP1 exclusively in BMSCs/SSCs attached to HyA-FMBs. Transcriptomic measurements confirmed increased BMP signaling in stable hyaline-like cartilage produced by ectopic transplantation of BMSCs/SSCs attached to HyA-FMBs.

A serum-free hiPSC differentiation strategy was developed that inhibited, then activated BMP signaling in a purified SOX9′ subpopulation that naturally detaches from monolayer cultures (termed “chondrospheroids”). Treatment of SOX9+ chondrospheroids with BMP-2 and GDF-5 produced uniform and stable expression of COL2A1, ACAN, and PRG4 and minimal expression of COL10A1 in vitro and in vivo.

Chondral transplantation of the disclosed unnatural chondrocytes, which mimicked the transcriptional identity of a fetal chondrocyte, produced stable hyaline-like cartilage for up to 5 months in NSG mice and SRG rats when attached to HyA-FMBs. Overall, hypertrophic-resistant chondrocytes were developed. These cells can be attached to a solid carrier and used to promote cartilage repair and growth.

### Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “a cell” includes singular or plural cells and can be considered equivalent to the phrase “at least one cell.” As used herein, the term “comprises” means “includes.” The term “about” indicates within five percent, unless the specific context states otherwise. It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Administration: To give a subject a therapeutic intervention, such as a therapeutic composition, procedure, or protocol (e.g., for a subject with a cartilage disease or injury). Routes of administration for a therapeutic composition include, but are not limited to injection (such as intra-articular injection).

Aggrecan: A molecule also referred to as cartilage-specific proteoglycan core protein. Aggrecan can be identified by a specific antibody as a marker for the presence of cartilage. UniProt Databank identified for human aggrecan is 4MD4. Aggrecan sequences are publicly available. For example, GENBANK® Accession Nos. NM_001135.3, NM_022190.1, NM_007424.2 disclose exemplary human, rat, and mouse aggrecan nucleotide sequences, respectively, and GENBANK® Accession Nos. NP_001126.3, NP_071526.1, NP_031450.2 disclose exemplary human, rat, and mouse aggrecan protein sequences, respectively. These GENBANK® entries are incorporated by reference as available on May 24, 2017. One of ordinary skill in the art can identify additional aggrecan nucleic acid and protein sequences, including isoform and transcript variants, peptide fragments, and peptides containing phosphorylation sites.

Alter: A change in an amount of a substance or parameter of interest, such as a polynucleotide, polypeptide or a property of a cell. An alteration in polypeptide or polynucleotide or activity can affect a physiological property of a cell, such as the differentiation, proliferation or survival of a cell. The amount of the substance can be changed by a difference in the amount of the substance produced, by a difference in the amount of the substance that has a desired function, or by a difference in the activation of the substance. The change can be an increase or a decrease. The alteration can be in vivo or in vitro. In several aspects, altering is at least about a 50%, 60%, 70%, 80%, 90%, 95%, 96%, 97%, 98%, 99%, or 100% increase or decrease in the amount (level) of differentiation, proliferation and/or survival of a cells, or in the amount of a specific protein or mRNA.

Animal: Living multi-cellular vertebrate organisms, a category that includes, for example, mammals and birds. The term mammal includes both human and non-human mammals. Similarly, the term “subject” includes both human and veterinary subjects, for example, non-human primates, dogs, cats, horses, rabbits, pigs, mice, rats, and cows.

Antagonist or Inhibitor: An agent that blocks or dampens a biochemical or biological response when bound to a receptor or a ligand of the receptor. Antagonists mediate their effects through receptor interactions by preventing agonist-induced responses. In one aspect, a Frizzled (Fzd) antagonist binds to a Fzd receptor or to a Fzd ligand (such as Wnt) and reduces or inhibits the Wnt/beta-catenin signaling pathway, for example a reduction of at least 50%, at least 60%, at least 70%, at least 80%, at least 90%, at least 95%, at least 96%, at least 97%, at least 98%, at least 99%, or 100%.

Attached: A term that indicates entities that are joined, conjugated, or combined, and includes covalent, ionic, polar or hydrogen bonds as well as the combination of materials, particles or molecules mixed together; e.g., fibrin microbeads mixed with hyaluronic acid.

Bone defect: Includes any disease, defect, or disorder which affects bone strength, function, and/or integrity, such as those resulting from injury, or a defect brought about during the course of surgery, infection, malignancy, or developmental malformation. Examples of bone defects include, but are not limited to, fractures (such as a microtrauma, a microfracture, or a subchondral fracture), dental or facial defects (such as cleft palate or facial, skull, or dental injuries or malformations). Other examples of bone defects include damage to bones resulting from diseases of bone fragility, such as osteoporosis, and malignancies and/or cancers of the bone such as a sarcoma, such as osteosarcoma.

Bone disease: Includes any disease or disorder which affects bone strength, function, and/or integrity, such as decreasing bone tensile strength and modulus. Examples of bone diseases include, but are not limited to, diseases of bone fragility and genetic diseases which result in abnormal bone formation. Bone diseases include, but are not limited to, osteogenesis imperfecta, osteoporosis, or a metabolic bone disease. Other examples of bone diseases include malignancies and/or cancers of the bone such as a sarcoma, such as osteosarcoma.

Bone Healing and Fracture Healing: Bone heals (fuses) in a unique way compared with other connective tissues. Rather than develop scar tissue, it has the innate ability to regenerate itself completely. Without being bound by theory, it is generally believed that the fracture healing sequence involves five discrete stages of healing. This includes an initial stage in which a hematoma is formed and inflammation occurs; a subsequent stage in which cartilage begins to form and angiogenesis proceeds, and then three successive stages of cartilage calcification, cartilage resorption and bone deposition, and ultimately a more chronic stage of bone remodeling. Generally, committed osteoprogenitor cells and uncommitted, undifferentiated skeletal stem cells contribute to the process of fracture healing. Generally, two weeks after fracture, cell proliferation declines and hypertrophic chondrocytes become the dominant cell type in the chondroid callus, and undergo further matrix mineralization, followed by infiltration of bone-forming cells. The resulting endochondral bone is formed adjacent to the fracture site.

Bone Marrow Stromal Cells (BMSCs), also referred to as Bone Marrow-Derived Mesenchymal “Stem Cells” or naïve BMSCs: A small fraction of cells in bone marrow, that occur in nature, and that are stem cell-like precursors for skeletal lineage including osteocytes, chondrocytes, and adipocytes and hematopoiesis supportive stroma. Bone marrow stromal cells have been studied extensively (Castro-Malaspina et al., 1980, Blood 56:289-30125; Piersma et al., 1985, Exp. Hematol 13:237-243; Simmons et al., 1991, Blood 78:55-62; Beresford et al., 1992, J. Cell. Sci. 102:341-351; Liesveld et al., 1989, Blood 73:1794-1800; Liesveld et al., Exp. Hematol 19:63-70; Bennett et al., 1991, J. Cell. Sci. 99:131-139). Bone marrow stromal cells can be derived from any animal. In some aspects, stromal cells are derived from primates, preferably humans. Human BMSCs are also referred to as hBMSCs. Naïve BMSCs refers to BMSCs that have not received a treatment to differentiate them, particularly to induce the BMSCs to form cartilage-forming cells, bone-forming cells, or an adipocyte-forming cells, see Satoma et al., J Cell Biochem. 2000 Jun. 6; 78(3):391-403, incorporated herein by reference. Naïve BMSCs are grown in standard culture conditions that do not enhance one cell phenotype over another (e.g. to promote differentiation into a cartilage-forming cell, a bone-forming cell, or an adipocyte-forming cell.)

BMSCs can be identified by expression of one or more of CD29, CD73, CD90, CD140b, and CD146, for example by using fluorescence activated cell sorting (FACS). In some aspects, BMSCs are autologous. In other aspects, BMSCs are allogeneic, as they are from a different animal of the same species. In aspects, BMSCs are naïve BMSCs.

Bone Morphogenetic Proteins (BMPs): A family of proteins, identified originally in extracts of demineralized bone that were capable of inducing bone formation at ectopic sites. BMPs are found in minute amounts in bone material (approximately 1 microgram/kg dry weight of bone). Most members of this family (with the exception of BMP-1) belong to the transforming growth factor-β family of proteins.

BMPs can be isolated from demineralized bones and osteosarcoma cells. They have been shown also to be expressed in a variety of epithelial and mesenchymal tissues in the embryo. BMPs are proteins which act to induce the differentiation of mesenchymal-type cells into chondrocytes and/or osteoblasts before initiating bone formation. They promote the differentiation of cartilage- and bone-forming cells near sites of fractures but also at ectopic locations. Some of the BMPs induce the synthesis of alkaline phosphatase and collagen in osteoblasts. Some BMPs act directly on osteoblasts and promote their maturation while at the same time suppressing myogenic differentiation. Other BMPs promote the conversion of mesenchymal cells into chondrocytes, and are also capable of inducing the expression of an osteoblast phenotype in non-osteogenic cell types. Among the BMPs, BMP-2 and BMP-4 and BMP-7 have been shown to promote bone and cartilage formation.

Bone Morphogenic Protein Receptor (BMPR): BMP receptors are a family of transmembrane serine/threonine kinases that include the type I receptors (BMPR1A and BMPR1B) and the type II receptor (BMPR2). These receptors are also closely related to the activin receptors, ACVR1 and ACVR2. The ligands of these receptors are members of the TGF beta superfamily. TGF-betas and activins transduce their signals through the formation of heterodimeric complexes with 2 different types of serine (threonine) kinase receptors: type I receptors of about 50-55 kD and type II receptors of about 70-80 kD. Type II receptors bind ligands in the absence of type I receptors, but they require their respective type I receptors for signaling, whereas type I receptors require their respective type H receptors for ligand binding. BMPs repress Wnt signaling to maintain stable stem cell populations. Exemplary RNA and protein sequences for a human BMPR1A receptor are disclosed in GENBANK® Accession No. NM_004329.3, Dec. 27, 2022, incorporated herein by reference.

Cartilage: A smooth, elastic tissue covering and protecting the ends of bones (e.g., at joints). Cartilage is composed of chondrocytes that produce a large amount of collagenous extracellular matrix, and is rich in proteoglycan and elastin fibers. The presence of cartilage can be determined by the expression of Type II Collagen, or Aggrecan. Hyaline cartilage, also referred to as hyaline-like cartilage, is a translucent or white cartilage containing little to no nerves or blood vessels, thereby limiting its repair capabilities. Articular cartilage is a type of hyaline cartilage that covers the ends of long bones in the joint region. The expression of the protein, Type X Collagen, indicates hypertrophic cartilage, which is present in developing long bones and metaphyseal bone marrow and in settings of fracture. Hypertrophic cartilage oftentimes serves as a template for endochondral bone formation and ossification. The methods of the present disclosure produce cartilage that persists in its cartilage state in vivo without vascularization or ossification. In aspects, the cartilage is stable for more than about 5 weeks, 6 weeks, 7 weeks, 8 weeks, 9 weeks, 10 weeks, 3 months, 6 months, 9 months, one year or more, after in vivo administration. Cartilage is radiolucent, however, clinically physicians can measure the distance between the honey epiphyses (joint space) as a surrogate to measure cartilage thickness.

Cartilage Injury: Any injury or damage to the cartilage tissue. Cartilage injuries include tears, rips and ruptures. Cartilage injury typically can affect the articular cartilage of joints e.g., knee, hip, wrist, elbow, shoulder, ankle, etc. Examples of other cartilage injuries to joint structures include meniscal tears, labral tears of the hip or shoulder and talar dome lesions, etc. Cartilage injury can result from chronic degenerative disease (e.g., osteoarthritis). Cartilage injuries can occur due to accident, athletic injury, or others.

Cluster of Differentiation (CD)5: A cluster of differentiation receptor that is typically associated with immune cells. Exemplary mRNA and amino acid sequences are disclosed in GENBANK® Accession Nos. NM_001346456.2, NP_001333385.1, Mar. 16, 2023, incorporated herein by reference.

CD27: A receptor, also referred to as TNFRSF7, associated with the Tumor Necrosis Factor (TNF)-receptor superfamily that typically binds the ligand CD70 to initiate an intracellular response. Exemplary mRNA and amino acid sequences are disclosed in GENBANK® Accession Nos. NM_001242.5, NP_001233.2, Mar. 16, 2023, incorporated herein by reference.

CD53: A cluster of differentiation receptor associated with the tetraspanin family that is typically involved in regulating cell development, growth, and motility, among others. Exemplary mRNA and amino acid sequences are disclosed in GENBANK® Accession Nos. NM_000560.4, NP_000551.1. Mar. 16, 2023, incorporated herein by reference.

Cell: A structural and functional unit of an organism that can replicate independently, is enclosed by a membrane, and contains biomolecules and genetic material. Cells used herein may be naturally-occurring cells or artificially modified cells (e.g., fusion cells, genetically modified cells, etc.).

The term “eel population” refers to a group of cells, typically of a common type. The cell population can be derived from a common progenitor or may comprise more than one cell type. An “enriched” cell population refers to a cell population derived from a starting cell population (e.g., an unfractionated, heterogeneous cell population) that contains a greater percentage of a specific cell type, such as chondrocytes, than the percentage of that cell type in the starting population. The cell populations may be enriched for one or more cell types and depleted of one or more cell types.

Cellular Adhesion Protein: A protein involved in the binding of a cell to other cells or in the extracellular matrix in the process called cellular adhesion. Cell adhesion proteins include vitronectin, fibrin and laminin, but also include the integrins, which mediates cell-ECM interactions with collagen, fibrinogen, fibronectin, and vitronectin, cadherins, which are homophilic calcium-dependent glycoproteins, and the selectins, which are a family of heterophilic proteins (E-selectin, L-selectin, and P-selectin) that are dependent on fucosylated carbohydrate.

Chondrocyte: A cell found in cartilage that produces and maintains the cartilaginous matrix, which consists of collagen and proteoglycans. In articular cartilage, the structure, density, and synthetic activity of an adult chondrocyte can vary according to the chondrocyte's position. Flattened cells are oriented parallel to the surface, along with the collagen fibers, in the superficial zone, the region of highest cell density. In the middle zone, chondrocytes are larger and more rounded and display a random distribution, in which the collagen fibers also are more randomly arranged. In the deeper zones, chondrocytes form columns that are oriented perpendicular to the cartilage surface, along with the collagen fibers. Different behaviors may be exhibited by chondrocytes depending on their position within the different layers. In primary chondrocyte cultures, these zonal differences in synthetic properties may persist. Endochondral ossification is the process by which most vertebrate axial skeletons form into hardened bones from chondrocytes. A “hypertrophic chondrocyte is about 10-20 fold larger in size than an articular chondrocyte and expresses Type X Collagen. A hypertrophic chondrocyte is committed to form bone or undergo apoptosis.

Chondrospheroid: A three-dimensional aggregate of cells differentiated from iPSCs that become loosely adherent or naturally detach from monolayer cultures after treatment with chondrogenic medium supplemented with TGFβ-superfamily ligand(s). Chondrospheroids can range from 1,000-1,000,000 cells per chondrospheroid, depending on the day of detachment from the monolayer (days 5-15 of chondrogenic induction). Chondrospheroids have higher expression of the master chondrogenic regulator, SRY-box transcription factor 9 (SOX9), compared with iPSCs. In some cases, entire monolayer cultures derived from iPSCs can detach and produce chondrospheroids that can be composed of up to 10,000,000 cells or more.

Collagen: The main structural protein in connective tissues of animals. Collagen may vary in its degree in formation of various tissues, for example, bone, tendon, and cartilage. There are several types of collagen which are present in varying degrees in different tissues. Collagen Types I-V are most common. Collagen Type II is the main collagenous component of cartilage.

Collagen type II: A collagen encoded by the COL2A1 gene. The UniProt identifier for human Collagen type II is P02458. Collagen type II sequences are publicly available. For example, GENBANK® Accession Nos. NM_001844.4, NM_012929.1, NM_031163.3 disclose exemplary human, rat, and mouse Collagen type II nucleotide sequences, respectively, and GENBANK® Accession Nos. NP_001835.3, NP_001835.3, NP_112440.2 disclose exemplary human, rat, and mouse Collagen type II protein sequences, respectively. These GENBANK® entries are incorporated by reference as available on May 24, 2017. One of ordinary skill in the art can identify additional Collagen Type II nucleic acid and protein sequences, including isoform and transcript variants, peptide fragments, and peptides containing phosphorylation sites.

Collagen Type X: A collagen encoded by the COL10A1 gene. The UniProt identifier for human Collagen type X is Q03692. Collagen type X sequences are publicly available. For example, GENBANK® Accession Nos. NM_000493.3, XM_001053056.7, NM_009925.4 disclose exemplary human, rat, and mouse Collagen type X nucleotide sequences, respectively, and GENBANK® Accession Nos. NP_000484.2, XP_001053056.5, NP_034055.1 disclose exemplary human, rat, and mouse Collagen type X protein sequences, respectively. These GENBANK® entries are incorporated by reference as available on May 24, 2017. One of ordinary skill in the art can identify additional Collagen Type X nucleic acid and protein sequences, including isoform and transcript variants, peptide fragments, and peptides containing phosphorylation sites.

Crosslinking: A process that is also referred to as bioconjugation, and is the process of covalently joining two or more molecules. Crosslinking reagents, or crosslinkers, contain two or more reactive ends capable of forming a covalent bond with a molecule of interest. Many chemical crosslinkers are available. Crosslinkers include, for example, 1-ethyl-3-(3-(dimethylamino) propyl) carbodiimide (EDC), or other cross-linker which can attach the HyA covalently to fibrin matrix without covalently with clearance of its residues after the reaction such as divinyl sulfone (DVS), glutaraldehyde (GTA), and/or poly(ethylene glycol) diglycidyl ether (EX 810).

Defined or Fully Defined: When used in relation to a medium, an extracellular matrix, or a culture condition, refers to a medium, an extracellular matrix, or a culture condition in which the chemical composition and amounts of approximately all the components are known. For example, a defined medium does not contain undefined factors such as in fetal bovine serum, bovine serum albumin or human serum albumin. Generally, a defined medium comprises a basal media (e.g., Dulbecco's Modified Eagle's Medium (DMEM), F12, or Roswell Park Memorial Institute Medium (RPMI) 1640, containing amino acids, vitamins, inorganic salts, buffers, antioxidants and energy sources) which is supplemented with recombinant albumin, chemically defined lipids, and recombinant insulin. An exemplary fully defined medium is ESSENTIAL 8™ medium.

Differentiation: The process by which an unspecialized cell becomes a more specialized type with changes in structural and/or functional properties. The mature cell typically has altered cellular structure and tissue-specific proteins. More specifically, in the context of the present methods indicates the process of a stem cell acquiring the cell characteristics of a chondrocyte.

Effective Amount: A quantity of a specific substance, such as a cells, for example chondrocytes derived from a hiPSC, sufficient to achieve a desired effect in a subject being treated. For instance, this can be the amount necessary to produce cartilage. When administered to a subject, a dosage will generally be used that will achieve target tissue concentrations that has been shown to achieve a desired in vitro effect, such as repair.

Essentially Free: In terms of a specified component, essentially free is used herein to mean that none of the specified component has been purposefully formulated into a composition and/or is present only as a contaminant or in trace amounts. The total amount of the specified component resulting from any unintended contamination of a composition is therefore well below 0.05%, such as below 0.01%. In some aspects, no amount of the specified component can be detected with standard analytical methods.

Expand: A process by which the number or amount of cells in a cell culture is increased due to cell division. Similarly, the terms “expansion” or “expanded” refer to this process. The terms “proliferate,” “proliferation” or “proliferated” may be used interchangeably with the words “expand,” “expansion”, or “expanded.” Typically, during an expansion phase, the cells do not differentiate to form mature cells, but divide to form more cells.

Expression: The production of mRNA encoding a specific protein, or the production of the specific protein, in a cell. In one embodiment, “expression” indicates the production of mRNA.

Fibrin: A fibrous, non-globular protein, which is the proteolytic product of fibrinogen. The UniProt identifier for human fibrinogen is P02671. Fibrinogen sequences are publicly available. For example, GENBANK® Accession Nos. NM_021871.3, NM_001008724.1, NM_001111048.2 disclose exemplary human, rat, and mouse fibrinogen nucleotide sequences, respectively, and GENBANK® Accession Nos. NP_068657.1, NP_001008724.1, NP_001104518.1 disclose exemplary human, rat, and mouse fibrinogen protein sequences, respectively. These GENBANK® entries are incorporated by reference as available on May 24, 2017. One of ordinary skill in the art can identify additional fibrinogen nucleic acid and protein sequences, including isoform and transcript variants, peptide fragments, and peptides containing phosphorylation sites.

Fibrin Microbeads (FMBs): Microparticles primarily composed of fibrin. Fibrin microbeads and methods of making the same are included in U.S. Pat. Nos. 6,552,172; 6,503,731; and 6,150,505; incorporated by reference herein in their entireties.

Fibrin microbeads can be produced from dense fibrin gels, which are vigorously mixed in heated oil to temperature of 60-85° C. to form a suspension. This suspension is further mixed vigorously in the oil for 4-10 hrs to form the dense dehydrated fibrin microbeads. The microbeads can be further collected washed and dried to yield the basic fibrin microbeads structure. The microbeads can be further condensed into the shape of separate beads by dehydrothermal crosslinking. Fibrin microbeads can have a density of greater than greater than 1.15 g/mL, for example from about 1.1-1.4 g/mL, 1.2-1.4 g/mL, or 1.25-1.35 g/mL. In aspects, fibrin microbeads contain 70% or more fibrin. The resultant solid fibrin microbeads can then be coated with Hyaluronic Acid.

Fibroblast growth factor (FGF): Any suitable fibroblast growth factor, derived from any animal, and functional fragments thereof, such as those that bind the receptor and induce biological effects related to activation of the receptor. Exemplary FGFs include, but are not limited to, FGF-1 (acidic fibroblast growth factor), FGF-2 (basic fibroblast growth factor, bFGF), FGF-3 (int-2), FGF-4 (hst/K-FGF), FGF-5, FGF-6, FGF-7, FGF-8, FGF-9 and FGF-98. “FGF” refers to a fibroblast growth factor protein such as FGF-1, FGF-2, FGF-4, FGF-6, FGF-8, FGF-9 or FGF-98, or a biologically active fragment or mutant thereof. The FGF can be from any animal species. In one aspect, the FGF is mammalian FGF, including but not limited to, rodent, avian, canine, bovine, porcine, equine and human. The amino acid sequences and method for making many of the FGFs are known.

The amino acid sequence of human bFGF and methods for its recombinant expression are disclosed in U.S. Pat. No. 5,439,818, herein incorporated by reference in its entirety. The amino acid sequence of bovine bFGF and various methods for its recombinant expression are disclosed in U.S. Pat. No. 5,155,214, herein incorporated by reference in its entirety. When the 146 residue forms are compared, their amino acid sequences are nearly identical, with only two residues that differ. Recombinant bFGF-2, and other FGFs, can be purified to pharmaceutical quality (98% or greater purity) using the techniques described in detail in U.S. Pat. No. 4,956,455.

An FGF inducer includes an active fragment of FGF. In its simplest form, the active fragment is made by the removal of the N-terminal methionine, using well-known techniques for N-terminal methionine removal, such as a treatment with a methionine aminopeptidase. A second desirable truncation includes an FGF without its leader sequence. Those skilled in the art recognize the leader sequence as the series of hydrophobic residues at the N-terminus of a protein that facilitate its passage through a cell membrane but that are not necessary for activity and that are not found on the mature protein. Human and murine bFGF are commercially available.

Fracture: A medical condition in which a bone is cracked or broken; a break in the continuity of a bone. Fractures may be classified as closed or open. A closed fracture is one in which the skin is intact; an open (or compound) fracture is one in which the bone is in contact with the air (such as piercing the skin or due to severe tissue injury). Fractures are also classified as simple or multi-fragmentary. A simple fracture occurs along only one line (such as splitting a bone into two pieces), while a multi-fragmentary fracture splits a bone into multiple pieces (such as three or more pieces). Other types of fracture include complete, incomplete, linear, transverse, oblique, compression, spiral, comminuted, and compacted fractures. Additional fractures include a critical defect (such as when part of a bone is lost or removed) and a non-union fracture (such as when the ends of the fracture are not in contact with each other). “Microfracture” (MFx) is a marrow stimulation technique achieved by subchondral bone perforation to recruit autologous bone marrow cells into a cartilage defect. The recruited cells differentiate into fibrochondrocytes, which fill and remodel the injured area to form a fibrocartilage clot. The clot is composed primarily of type I collagen and is different from the native hyaline cartilage, which contains a large amount of type H collagen.

Growth Differentiation Factor (GDF)-5: A protein encoded by the GDF5 gene, closely related to bone morphogenetic proteins (BMP) and is a member of the TGF-β superfamily. An amino acid sequence and mRNA sequence encoding human GDF5 is provided in GENBANK® Accession No. NP_001306067.1, Dec. 27, 2022, incorporated by reference herein.

Growth: An increase in the amount of a tissue, for example cartilage. Growth can be assessed by expansion in size, weight, or progression of tissue development.

Growth factor: A substance that promotes cell growth, survival, and/or differentiation. Growth factors include molecules that function as growth stimulators (mitogens), factors that stimulate cell migration, factors that function as chemotactic agents or inhibit cell migration or invasion of tumor cells, factors that modulate differentiated functions of cells, factors involved in apoptosis, or factors that promote survival of cells without influencing growth and differentiation. Examples of growth factors are a fibroblast growth factor (such as FGF-2), epidermal growth factor (EGF), and activin-A.

Hyaluronic Acid (HyA), also referred to as hyaluronan or HA, is an anionic, nonsulfated glycosaminoglycan distributed widely throughout connective, epithelial, and neural tissues. HyA has a monomeric structure as shown below:

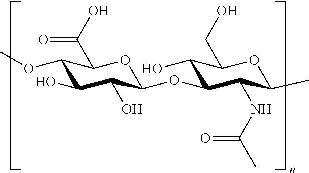

HyA can be high molecular weight hyaluronic acid, for example with an estimated size range of about 50,000-200,000 Da.

Hypertrophy: An increase in the size of cells. Type X collagen is a marker for chondrocyte hypertrophy. Another marker of hypertrophy is collagenase-3 or MMP13. Chondrocyte hypertrophy oftentimes precedes endochondral bone formation and ossification. Hypertrophic chondrocytes are an unwanted type of cell in adult humans, formed in cartilage as a result of aging or due to a disease process, such as osteoarthritis. Hypertrophic chondrocytes ultimately lead to the replacement of cartilage by bone, and loss of joint function. Hypertrophic chondrocytes can be detected by histological methods, such as toluidine blue staining.

Intra-articular or Intraarticular: Within a joint; e.g., an intra-articular procedure or injection. In aspects, an intra-articular procedure is within a hip, wrist, elbow, knee, shoulder, ankle joint, or others. In aspects, intra-articular injection can be ultrasound guided. Intra-articular can also refer to a minimally invasive surgical procedure; e.g., arthroscopic surgery, which can address intra-articular problems.

Isolated: An “isolated” cell has been substantially separated or purified from other cells in an organism or culture. Isolated cells can be, for example, at least 99%, at least 98% pure, at least 95% pure or at least 90% pure.

Mammal: This term includes both human and non-human mammals. Examples of mammals include but are not limited to: humans and veterinary and laboratory animals, such as pigs, cows, goats, cats, dogs, rabbits and mice.

Medium: A synthetic set of culture conditions with the nutrients necessary to support the growth (cell proliferation/expansion) and/or differentiation of a specific population of cells. In one aspect, the cells are stem cells, such as iPSCs. In another aspect, the cells are chondrocytes. Media generally include a carbon source, a nitrogen source and a buffer to maintain pH. In one aspect, growth medium contains a minimal essential media, such as DMEM, supplemented with various nutrients to enhance stem cell growth.

Additionally, the minimal essential media may be supplemented with additives such as horse, calf or fetal bovine serum.

Microbead: A solid support in the form of a bead that is generally spherical, is generally biocompatible, and has a diameter of about 40-300 μm.

Myogenin (MYOG): A helix-loop-helix transcription factor that is typically associated in regulating muscle development. Exemplary mRNA and amino acid sequences are disclosed in GENBANK® Accession Nos. NM_002479.6, NP_002470.2, Mar. 16, 2023, incorporated herein by reference.

Non-natural: A cell or component that has a phenotype that does not occur in nature, and is not a wild-type cell. In some aspects, a non-natural cell can express a recombinant protein or include a nucleic acid molecule that is produced by genetic engineering. Non-natural cell, such as a chondrocyte, can be produced from iPSC, and have at least one marker that is expressed significantly differently from a cell, such as a chondrocyte or hypertrophic chondrocyte. The expression of the marker can be increased or decreased, as compared to the same type of natural cells, or can be newly expressed, as compared to the same type of natural cells.

Osteoarthitis: A type of joint disease resultant from the breakdown of cartilage and bone within the joints. Osteoarthritis causes joint pain and stiffness, swelling and decreased range of motion. The cartilage covering bones (articular cartilage—a subset of hyaline cartilage) is thinned, eventually completely wearing away, resulting in a “bone against bone” within the joint, leading to reduced motion, and pain. In some cases, aberrant bone formation can occur (e.g., osteophytes).

Pharmaceutically acceptable carriers: Conventional pharmaceutically acceptable carriers are useful for practicing the methods and forming the compositions disclosed herein. Remington's Pharmaceutical Sciences, by E. W. Martin, Mack Publishing Co., Easton, PA, 15th Edition, 1975, describes examples of compositions and formulations suitable for pharmaceutical delivery of the compounds herein disclosed.

In general, the nature of the carrier will depend on the particular mode of administration being employed. For example, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Repair: The regrowth of tissue, for example, the repair of torn or degraded cartilage following injury or disease. Repair does not necessarily indicate full restoration to a pre-injury, or pre-disease state. Repair can include partial repair, for example about 10%, 20%, 30%, 40%, 50%, 60%, 70%, 80%, 90%, 95%, 99%, or more regrowth in injured tissue. Repair can be measured as a percentage extension in length, height, width, diameter, or weight. Repair can further be measured as an increase in joint mobility, or subjectively, as in increase in joint comfort.

Sclerotome: Monolayer cells derived from iPSCs or embryonic stem cells that have similar expression patterns (e.g., PAX1, PAX9, NKX3.2/BAPX1, and FOXC2A) to cells of developing human mesoderm cell populations mostly located in the ventromedial region of the somite.

Secondary Ossification Center Associated Regulator Of Chondrocyte Maturation (SNORC): A small membrane proteoglycan that is enriched in chondrocytes. Exemplary mRNA and amino acid sequences are disclosed in GENBANK® Accession Nos. NM_001346120.3, NP_001333049.2, Mar. 16, 2023, incorporated herein by reference.

Stem Cell: A cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent stem cell, multipotent stem cell, precursor cell and progenitor cell. Exemplary human stem cells can be obtained from hematopoietic or skeletal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPSCs.” A human iPSC is denoted “hiPSC.”

An “embryonic stem cell (ESC)” is an undifferentiated pluripotent cell which is obtained from an embryo in an early stage, such as the inner cell mass at the blastocyst stage, or produced by artificial means (e.g., nuclear transfer) and can give rise to any differentiated cell type in an embryo or an adult, including germ cells (e.g., sperm and eggs). The term includes cells isolated from one or more blastomeres of an embryo, preferably without destroying the remainder of the embryo. The term also includes cells produced by somatic cell nuclear transfer. “Human embryonic stem cells” (hESCs) includes embryonic cells derived from the inner cell mass of human blastocysts or morulae, optionally that have been serially passaged as cell lines. ESCs may be derived from fertilization of an egg cell with sperm or DNA, nuclear transfer, parthenogenesis, or by means to generate hESCs with homozygosity in the HLA region. hESCs can be produced or derived from a zygote, blastomeres, or blastocyst-staged mammalian embryo produced by the fusion of a sperm and egg cell, nuclear transfer, parthenogenesis, or the reprogramming of chromatin and subsequent incorporation of the reprogrammed chromatin into a plasma membrane to produce an embryonic cell. hESCs include, but are not limited to, MAO1, MAO9, ACT-4, No. 3, H1, H7, H9, H14 and ACT30 embryonic stem cells. hESCs, regardless of their source or the particular method used to produce them, can be identified based on (i) the ability to differentiate into cells of all three germ layers, (ii) expression of at least Oct-4 and alkaline phosphatase, and (iii) ability to produce teratomas when transplanted into immunocompromised animals.

“Induced pluripotent stem cells (iPSCs)” are cells generated by reprogramming a somatic cell by expressing or inducing expression of a combination of factors (herein referred to as reprogramming factors). iPSCs can be generated using fetal, postnatal, newborn, juvenile, or adult somatic cells. In certain aspects, factors that can be used to reprogram somatic cells to pluripotent stem cells include, for example, Oct4 (sometimes referred to as Oct 3/4), Sox2, c-Myc, and Klf4, Nanog, and Lin28. In some aspects, somatic cells are reprogrammed by expressing at least two reprogramming factors, at least three reprogramming factors, or four reprogramming factors to reprogram a somatic cell to a pluripotent stem cell.

The term “pluripotent” refers to the property of a cell to differentiate into all other cell types in an organism, with the exception of extraembryonic, or placental, cells. Pluripotent stem cells are capable of differentiating to cell types of all three germ layers (e.g., ectodermal, mesodermal, and endodermal cell types) even after prolonged culture. A pluripotent stem cell is an embryonic stem cell derived from the inner cell mass of a blastocyst. In other aspects, the pluripotent stem cell is an induced pluripotent stem cell derived by reprogramming somatic cells.

Subject: Includes both human and veterinary subjects, such as humans, non-human primates, pigs, sheep, cows, rodents, birds, and the like, which can be the recipient of the disclosed methods. An “animal” is a living, multi-cellular vertebrate organisms, a category that includes, for example, mammals and birds (e.g., chickens). The term mammal includes both human and non-human mammals. In two non-limiting examples, a subject is a human subject or a murine subject.

Tumor necrosis factor receptor superfamily member 1B (TNFRSF1B): A receptor, also referred to as CD120b, associated with the Tumor Necrosis Factor (TNF)-receptor superfamily that has implications in regulating apoptosis. Exemplary mRNA and amino acid sequences are disclosed in GENBANK® Accession Nos. NM_001066.3, NP_001057.1, Mar. 16, 2023, incorporated herein by reference.

Transforming Growth Factor (TGF)-β: A molecule that interacts with the TGFβ receptor. TGF-β is a multifunctional set of peptides that controls proliferation, differentiation, and other functions in many cell types. TGF-β acts synergistically with transforming growth factor-alpha (TGF-α) in inducing transformation. It also acts as a negative autocrine growth factor. Dysregulation of TGF-β activation and signaling may result in apoptosis. Many cells synthesize TGF-β and almost all of them have specific receptors for this peptide. TGF-β1, TGF-β2, and TGF-β3 all function through the same receptor signaling systems.

TGF-β1 is a peptide of 112 amino acid residues derived by proteolytic cleavage from the C-terminal of a precursor protein. TGFs interact with a conserved family of cell surface serine/threonine-specific protein kinase receptors, and generate intracellular signals using SMADs. Proteins from the TGF-beta superfamily are only active as homo- or heterodimer; the two chains being linked by a single disulfide bond. Exemplary amino acid and mRNA sequences are disclosed in GENBANK® Accession No. NM_000660.7, Feb. 19, 2023, incorporated herein by reference.

TGF-β3 is a peptide formed as a preproprotein of 412 amino acids in length. Exemplary amino acid and mRNA sequences are disclosed in GENBANK® Accession No. NC_000014.9, Feb. 25 2022, incorporated herein by reference.

Treatment: Therapeutic measures that cure, slow down, lessen symptoms of, inhibit and/or halt progression of a diagnosed pathologic condition or disorder. A “therapeutically effective” amount of a compound or a cell, such as a chondrocyte, that, when administered to a subject for treatment of a disease or condition, is sufficient to affect such treatment, or to reduce a symptom of the disease or condition, such as a fracture.

Undifferentiated: Cells that display characteristic markers and morphological characteristics of undifferentiated cells, distinguishing them from differentiated cells of embryo or adult origin. Thus, in some aspects, undifferentiated cells do not express cell lineage specific markers, including, but no limited to, chondrocytes.

Wnt: A family of highly conserved secreted signaling molecules that regulate cell-to-cell interactions and are related to the Drosophila segment polarity gene, wingless. In humans, the Wnt family of genes encodes 38 to 43 kDa cysteine rich glycoproteins. The Wnt proteins have a hydrophobic signal sequence, a conserved asparagine-linked oligosaccharide consensus sequence (see e.g., Shimizu et al Cell Growth Differ 8:1349-1358 (1997)) and 22 conserved cysteine residues. Because of their ability to promote stabilization of cytoplasmic beta-catenin, Wnt proteins can act as transcriptional activators and inhibit apoptosis. Overexpression of particular Wnt proteins is associated with certain cancers.

The Wnt family contains at least 19 mammalian members. Exemplary Wnt proteins include Wnt-1, Wnt-2, Wnt2b, Wnt-3, Wnt-3a, Wnt-4, Wnt-5a, Wnt5b, Wnt-6, Wnt-7a, Wnt-7b, Wnt-8a, Wnt-8b, Wnt9a, Wnt9b, Wnt10a, Wnt-10b, Wnt-11, and Wnt 16. These secreted ligands activate at least three different signaling pathways. In the canonical (or Wnt/beta-catenin) Wnt signaling pathway, Wnt activates a receptor complex consisting of a Frizzled (Fzd) receptor family member and low-density lipoprotein (LDL) receptor-related protein 5 or 6 (LRP5/6). To form the receptor complex that binds the Fzd ligands, Fzd receptors interact with LRP5/6, single pass transmembrane proteins with four extracellular EGF-like domains separated by six YWTD amino acid repeats (Johnson et al., 2004, J. Bone Mineral Res. 19:1749). The canonical Wnt signaling pathway activated upon receptor binding is mediated by the cytoplasmic protein Disheveled (Dvl) interacting directly with the Fzd receptor and results in the cytoplasmic stabilization and accumulation of beta-catenin. In the absence of a Wnt signal, beta-catenin is localized to a cytoplasmic destruction complex that includes the tumor suppressor proteins adenomatous polyposis coli (APC) and Axin. These proteins function as critical scaffolds to allow glycogen synthase kinase (GSK)-3beta to bind and phosphorylate beta-catenin, marking it for degradation via the ubiquitin/proteasome pathway. Activation of Dvl results in the dissociation of the destruction complex. Accumulated cytoplasmic beta-catenin is then transported into the nucleus where it interacts with the DNA-binding proteins of the TCF/LEF family to activate transcription.

The non-canonical WNT pathway is regulated by three of these WNT ligands—WNT4, WNT5a, and WNT11. These ligands bind to the WNT receptor Frizzled in the absence of the co-receptors (LRP5/6). This leads to the activation of the RHO GTPase and ROCK kinase without activating cytoplasmic beta-catenin. ROCK regulates cytoskeleton to regulate apical-basal polarity of the cell. Because of competition for the same receptor, non-canonical WNT ligands also lead to inhibition of canonical WNT signaling.

All publications, patent applications, patents, and other references mentioned herein are incorporated by reference in their entirety for all purposes. All sequences associated with the GENBANK® Accession numbers mentioned herein are incorporated by reference in their entirety as were present on Mar. 17, 2023, to the extent permissible by applicable rules and/or law.

Chondrocytes Derived from hiPSC

Isolated non-natural human chondrocytes are disclosed herein that are derived from hiPSC. These non-natural human chondrocytes do not undergo hypertrophy for at least 42 days in vitro. In some aspects, the non-natural chondrocytes do not undergo hypertrophy for a at least about 42, 45, 50, 55, 60, 65, 70 or 75 days in vitro. In more aspects, the non-natural chondrocytes do not undergo hypertrophy for about 42, 45, 50, 55, 60, 65, 70 or 75 days in vitro. In some aspect, the non-natural human chondrocyte does not undergo hypertrophy for at least 5 months when transplanted in vivo. In some aspects, the non-natural chondrocytes do not undergo hypertrophy for a at least about 5, 6, 7, 8, 9, 10, 11 or 12 months in vivo. In more aspects, the non-natural chondrocytes do not undergo hypertrophy for a at least 5, 6, 7, 8, 9, 10, 11 or 12 months in vivo.

In some aspects, the non-natural human chondrocyte has decreased expression of Type X Collagen (COL10A1) as compared to a hypertrophic chondrocyte. In some aspects, the non-natural human chondrocyte that has similar expression of type II collagen (COL2A1), Aggrecan (ACAN), and Proteoglycan (PRG)4 as compared to wild-type human adult and fetal articular chondrocytes. In some aspects, COL10A1 mRNA is decreased I the non-natural human chondrocyte as compared to a hypertrophic chondrocyte.

In some aspects, expression of at least one of SNORC, LUZP2, and STMN2 are increased as compared to adult and fetal human chondrocytes. In more aspects, expression of at least two of SNORC, LUZP2, and STMN2 are increased as compared to adult and fetal human chondrocytes. In a non-limiting example, expression of all of SNORC, LUZP2, and STMN2 are increased in the non-natural human chondrocytes as compared to adult and fetal human chondrocytes. In some aspects, SNORC, LUZP2, and/or STMN2 mRNA is increased in the non-natural human chondrocyte as compared to adult and fetal human chondrocytes.

In some aspects, expression of at least one of CD5, CD27, CD53, TNFRSF1B, and MYOG are decreased in the non-natural human chondrocytes as compared to adult and fetal human chondrocytes. In more aspects, at least two of CD5, CD27, CD53, TNFRSF1B, and MYOG are decreased in the non-natural human chondrocytes as compared to adult and fetal human chondrocytes. In further aspects, at least three of CD5, CD27, CD53, TNFRSF1B, and MYOG are decreased as compared to adult and fetal human chondrocytes. In other aspects, expression of at least four of CD5, CD27, CD53, TNFRSF1B, and MYOG are decreased in the non-natural human chondrocytes as compared to adult and fetal human chondrocytes. In a non-limiting example, all of CD5, CD27, CD53, TNFRSF1B, and MYOG are decreased as compared to adult and fetal human chondrocytes. In some aspects, CD5, CD27, CD53, TNFRSF1B, and/or MYOG mRNA is decreased in the non-natural human chondrocyte as compared to adult and fetal human chondrocytes.

In some aspects, in the non-natural human chondrocytes, expression of at least one of SNORC, LUZP2, and STMN2 are increased as compared to adult and fetal human chondrocytes, and expression of at least one of CD5, CD27, CD53, TNFRSF1B, and MYOG are decreased as compared to adult and fetal human chondrocytes. In other aspects, in the non-natural human chondrocytes, expression of all of SNORC, LUZP2, and STMN2 are increased as compared to adult and fetal human chondrocytes, and expression of all of CD5, CD27, CD53, TNFRSF1B, and MYOG are decreased as compared to adult and fetal human chondrocytes. In some aspects, SNORC, LUZP2, and/or STMN2 mRNA is increased in the non-natural human chondrocyte as compared to adult and fetal human chondrocytes. In more aspects, CD5, CD27, CD53, TNFRSF1B, and/or MYOG mRNA is decreased in the non-natural human chondrocyte as compared to adult and fetal human chondrocytes.

Chondrospheroids are also provided that include non-natural human chondrocytes derived from an iPSC, as disclosed herein. A chondrospheroid can include for example, about 1,000 to about 1,000,000 chondrocytes, such as about 1,000, about 10,000, about 100,000 or about 1,000,000 chondrocytes. A chondrosphere can include for example, about 10,000, about 20,000, about 30,000, about 40,000, about 50,000, about 60,000, about 70,000, about 80,000, or about 90,000 chondrocytes. A chondrosphere can include for example, about 100,000, about 200,000, about 300,000, about 400,000, about 500,000, about 600,000, about 700,000, about 800,000, or about 900,000 chondrocytes. Chondrospheroids can be digested with an enzyme to release single chondrocytes. Suitable enzymes include, but are not limited to, collagenase, dispase, liberase, or trypsin. Exemplary methods are provided in the examples section.

Non-natural chondrocytes can be derived from hiPSCs, and then can be cryopreserved, see for example, PCT Publication No. 2012/149484 A2, which is incorporated by reference herein in its entirety, and discloses cryopreservation methods. The cells can be cryopreserved with or without a substrate. In several aspects, the storage temperature ranges from about −50° C. to about −60° C., about −60° C. to about −70° C., about −70° C. to about −80° C., about −80° C. to about −90° C., about −90° C. to about −100° C., and overlapping ranges thereof. In some aspects, lower temperatures are used for the storage (e.g., maintenance) of the cryopreserved cells. In several aspects, liquid nitrogen (or other similar liquid coolant) is used to store the cells. In further aspects, the cells are stored for greater than about 6 hours. In additional aspects, the cells are stored about 72 hours. In several aspects, the cells are stored 48 hours to about one week. In yet other aspects, the cells are stored for about 1, 2, 3, 4, 5, 6, 7, or 8 weeks. In further aspects, the cells are stored for 1, 2, 3, 4, 5, 67, 8, 9, 10, 11 or 12 months. The cells can also be stored for longer times. The cells can be cryopreserved separately or on a substrate, such as any of the substrates disclosed herein.

In some aspects, additional cryoprotectants can be used. For example, the cells can be cryopreserved in a cryopreservation solution comprising one or more cryoprotectants, such as DM80, serum albumin, such as human or bovine serum albumin. The cryoprotectant can intercalate into the cell membrane and change the properties of the cells so that it survives freezing. Chondrocytes can be cryopreserved as isolated populations, or can be mixed with other cells of interest prior to cryopreservation.

In certain aspects, the solution includes about 1%, about 1.5%, about 2%, about 2.5%, about 3%, about 4%, about 5%, about 6%, about 7%, about 8%, about 9%, or about 10% DMSO. In other aspects, the solution includes about 1% to about 3%, about 2% to about 4%, about 3% to about 5%, about 4% to about 6%, about 5% to about 7%, about 6% to about 8%, about 7% to about 9%, or about 8%. to about 10% dimethylsulfoxide (DMSO) or albumin. In a specific aspect, the solution includes 2.5% DMSO. In another specific aspect, the solution includes 10% DMSO.

Cells may be cooled, for example, at about 1° C. minute during cryopreservation. In some aspects, the cryopreservation temperature is about −80° C. to about −180° C., or about −125° C. to about −140° C. In some aspects, the cells are cooled to 4° C. prior to cooling at about 1° C./minute. Cryopreserved cells can be transferred to vapor phase of liquid nitrogen prior to thawing for use. In some aspects, for example, once the cells have reached about −80° C., they are transferred to a liquid nitrogen storage area. Cryopreservation can also be done using a controlled-rate freezer. Cryopreserved cells may be thawed, e.g., at a temperature of about 25° C. to about 40° C., such as at a temperature of about 37° C.

Pharmaceutical compositions including a therapeutically effective amount of the chondrocytes derived from a hiPSC are also of use in the method disclosed herein. The composition can also contain additional components. such as osteoinductive factors. Such osteoinductive factors include, for example, dexamethasone, ascorbic acid-2-phosphate, β-glycerophosphate and/or transforming growth factor (TGF) superfamily proteins, such as the bone morphogenetic proteins (BMPs). The composition can also contain antibiotic, antimycotic, anti-inflammatory, immunosuppressive and other types of therapeutic, preservative and excipient agents.

### Conjugates

The disclosed non-natural chondrocytes derived from hiPSC can be conjugated to a solid carrier. Solid carriers include, but are not limited to collagen, fibrin, gelatin, hyaluronic acid and hydroxyapatite.

A variety of biological or synthetic solid matrix materials (i.e., adhesives or dressings, biological/medical scaffolds, microbeads, etc.) are suitable for use with the present cells. The material can be biodegradable or non-biodegradable. Any transplantable solid surface can be utilized.

The material is generally physiologically acceptable and suitable for use in vivo applications. Non-limiting examples of such physiologically acceptable materials include, but are not limited to, solid matrix materials that are biodegradable. such crosslinked or non-crosslinked alginate, hydrocolloid, foams, collagen gel, collagen sponge, polyglycolic acid (PGA) mesh, polyglactin (PGL) mesh, and bioadhesives (e.g., fibrin glue and fibrin gel). The polymer can be poly(DL)-lactic-co-glycolic) acid (PLGA) (see Lu et al., J. Biomater Sci Polym Ed. 9(11): 1187-205, 1998). In other aspects, the matrix includes poly(L-lactic acid) (PLLA) and poly(D,L-lactic-co-glycolic acid) (PLGA), such as with a co-polymer ratio of about 90:10, 75:25, 50:50, 25:75, 10:90 (PLLA:PLGA) (see Thomson et al., J. Biomed. Mater Res. A 95: 1233-42, 2010).

Suitable polymeric carriers include porous meshes or sponges formed of synthetic or natural polymers. Natural polymers that can be used include proteins such as collagen, albumin, and fibrin; and polysaccharides such as alginate and polymers of hyaluronic acid. Synthetic polymers can be biodegradable. Examples of biodegradable polymers include polymers of hydroxy acids such as polylactic acid (PLA), polyglycolic acid (PGA), and polylactic acid-glycolic acid (PLGA), polyorthoesters, polyanhydrides, polyphosphazenes, and combinations thereof.

In some aspects, the scaffold is a PLGA scaffold. PLGA is a copolymer of poly-lactic acid (PLA) and poly-glycolic acid (PGA). Poly-lactic acid contains an asymmetric α-carbon which is typically described as the D or L form in classical stereochemical terms and sometimes as R and S form, respectively. The enantiomeric forms of the polymer PLA are poly D-lactic acid (PDLA) and poly L-lactic acid (PLLA). PLGA is poly D, L-lactic-co-glycolic acid where D- and L-lactic acid forms are generally in equal ratio. PLGA biodegrades by hydrolysis of its ester linkages. In some aspects, the PLGA scaffold is cultured for a sufficient time such that the bulk of lactic acid release from the scaffold occurs in vitro. In some aspects, greater than 50%, 60%, 70%, 80%, 90% or 95% of the lactic acid release occurs in vitro. The lactic acid release occurs over time. In some aspects, the PLGA scaffold has a DL-lactide/glycotide ratio of about 5:1 to about 1:5, such as about 4:1 to about 1:4, about 3:10 to about 3:3, about 2:1 to 1:2. In one specific non-limiting examples, the DL-lactide/glycotide ratio is 1:1.

The solid carrier can include nanofibers that intersect each other, such that they intersect and form junctions. The solid carrier can be treated to fuse fibers at the junctions of fiber intersections within the PLGA scaffold to increase mechanical strength. The average pore size is the space between the fibers in the PLGA scaffold.

In some aspects, the solid carrier is a fibrin microbead. See U.S. Pat. No. 10,940,241, incorporated herein by reference, which discloses fibrin microbeads and methods for cross-linking cells to fibrin microbeads. Fibrin microbeads are microparticles primarily composed of fibrin. Fibrin is a proteolytic product of fibrinogen, a glycoprotein. Fibrin microbeads and methods of making the same are included in U.S. Pat. Nos. 6,552,172; 6,503,731; and 6,150,505; incorporated by reference herein in their entireties.

In some aspects, fibrin microbeads have a density of greater than greater than 1.15 g/mL, for example from about 1.1 to about 1.4 g/mL, about 1.2 to about 1.4 g/mL, or about 1.25 to about 1.35 g/mL. In further aspects, fibrin microbeads contain 70% or more fibrin, for example 71% fibrin, 72% fibrin, 73% fibrin, 74% fibrin, 75% fibrin, or more. Without being bound by theory, the dense structure and mechanical stability of fibrin microbeads resists vascularization and allows for stable cartilage formation from bone marrow stromal cells in vivo. In aspects, microbeads have a diameter of about 40-300 μm, or about 60-250 μm.

In some aspects, an aqueous solution comprising fibrinogen, thrombin and factor XIII is prepared, such as by combining fibrinogen containing endogenous factor XIII with thrombin, by combining cryoprecipitate containing endogenous fibrinogen and endogenous factor XIII with thrombin, or by combining fibrinogen, factor XIII and thrombin individually into an aqueous solution. Alternatively, sources containing blood plasma fractionation products of procedures which enriches their fibrinogen concentration, such as cryo-precipitate (“paste 1”) with fibrinogen concentration above 10 mg/ml, including crude could also be used as the source of the fibrinogen. Equivalent fibrinogen activating proteases such as snake venom proteases (e.g., reptilase) can be used as an alternative to thrombin. In some aspects, the ratio of fibrinogen:thrombin:factor XIII in the aqueous solution is about 5 to about 100 mg/mL, about 1 to about 100 U/mL, about 1 to about 50 U/mL, about 20 to about 40 mg/mL, about 5 to about 10 U/mL, or about 2 to about 20 U/mL. In addition, the aqueous solution also can contain co precipitating proteins such as fibronectin and other blood-derived proteins that may be present in the rich fibrinogen solution and cryoprecipitate starting materials.

The aqueous solution with fibrinogen and activating protease, such as thrombin, immediately after their mixing is introduced into a very fast mixed and stirred oil heated to a temperature in the range of 60 to about 85° C. to form an emulsion with continuous fast stirring. Any hydrophobic organic solvent such as isooctane also may be included in the oil. The suspension is mixed in the oil for about 3 to about 10 hours, such as about 4 to about 10 hours. The mixing speed will depend upon the volume of the emulsion, and the desired size of the microbeads. For example, volumes of >400 mL oil and about 100 mL aqueous phase in a 1 L flask, an exemplary vigorous mixing speed is at least 300-500 rpm.

Fibrin microbeads can be isolated from the emulsion using procedures such as centrifugation, filtration, rinsing in different organic solvents and alcohols or a combination thereof. The isolated fibrin microbeads may can be washed with solvents, such as, but not limited to, hexane, acetone and/or ethanol and ether, and then air dried in ambient temperature moderately heated in atmospheric pressure or in vacuum heated or no heated condition. The microbeads may then be graded to the desired size using commercially available filters or sieves. Preferably, the fibrin microbeads are graded to a diameter of about 80-250 microns, although larger or smaller fibrin microbeads may be utilized. In some aspects, the microbeads can be further condensed by dehydrothermal cross-linking, either before or after grading them into fractions of the desired size. Not to be bound by theory, the long time stirring in heated oil results with full dehydration of the small hydrous fibrin gel initially containing hydrous droplets which dry slowly and eventually results in the spontaneous dehydrothermal non-reversible covalent crosslinking of the proteins by the long exposure of many hours to the ambient moderately high temperature.

In some non-limiting examples, fibrin microbeads can be produced from dense fibrin gels which are vigorously mixed in heated oil to temperature of 60-80° C. to form a suspension. This suspension is further mixed vigorously in the oil for 4-10 hours to form the dense dehydrated fibrin microbeads. The microbeads can be further collected washed and dried to yield the basic fibrin microbeads structure. The microbeads can be further condensed into the shape of separate heads by dehydrothermal crosslinking. Protocols are disclosed for example, in Gorodetsky et al., J Invest Dematol. 1999; 112(6):866-72 and Gorodetsky, Expert Opin Biol Ther. 2008; 8(12):1831-46, both incorporated herein by reference, in their entireties.

In other non-limiting examples, frozen plasma-derived fibrinogen-enriched solution was purified by sedimentation. A concentrated solution of about 40-80 mg/mL, about 50-70 mg/mL, or about 55-65 mg/mL of clotable, soluble protein was obtained. To form FMBs, the fibrinogen solution was mixed with thrombin/Ca+2 to reach a final concentration of about 0.5-10U/mL, about 1-8U/mL, or about 1-6 U/mL thrombin and about 2-6 mM Ca⁺², or about 4 mM Ca⁺². Upon initiation of coagulation, the mixture was immediately poured into a heat-stable oil; e.g., pure medium-chain-triglycerides oil (MCT, Edomim-Food Supplements, Israel) or any other similar oxidation resistant oil heated to reach a temperature of about 60-85° C., in a heavy-duty mixer attached to a temperature controlled heater. An emulsion with small, concentrated fibrin gel droplets floating as a suspension in the oil, was formed and stabilized within about 15 min-1 hour, about 20-50 min, or about 30-45 min. Following an additional about 4-10 hrs, about 5-9 hours, or about 6-8 hrs of continuous mixing at high speed, condensed, dehydrothermally stabilized FMBs are formed in the heated oil. The resulting solid FMBs are collected and thoroughly washed to remove oil residue by a series of rinses; e.g., initially with hexan, then with acetone, followed by final rinses in an ethanol gradient of 70%, 96% and 100%. Dried FMBs are mesh-sieved, and the size range between about 50-250 μm, about 80-200 μm, or about 105-180 μm are collected and stored at room temperature (RT) for further use. FMB can be stored for a period of greater than 5 years without notable detriment.

Fibrin microbeads can be coated with (e.g., crosslinked to) hyaluronic acid (HyA). In aspects, fibrin microbeads can be coated with HyA using a crosslinking agent, for example divinyl sulfone (DVS), glutaraldehyde (GTA), poly(ethylene glycol) diglycidyl ether (EX 810), and/or EDC. In aspects, HyA can be mixed, for example by shaking, with a solution of fibrin microbeads, about 30 minutes to about 6 hours, such as for about 30 minutes, about 1 hour, about 2 hours, about 3 hours, about 4 hours, about 5 hours, or 6 hours in the presence of a cross-linking agent. Longer times intervals could also be used. Following mixing, the fibrin microbeads with crosslinked HyA can be isolated. In one aspect, they are allowed to settle and the supernatant removed for rinsing in series of organic solvents and resuspension to rinse away the residual oil, drying in air or vacuum in ambient or moderate heating in up to ~70° C.

In one, non-limiting example, modification of FMBs with hyaluronic acid to generate HyA-FMBs is performed by a covalent reaction with the crosslinker, 1-ethyl-3-(3-(dimethylamino) propyl) carbodiimide (EDC). About 20-60 mg, about 30-50 mg, or about 40 mg EDC is added to about 400 to about 900 mg, about 500 to about 700 mg, or about 600 mg of FMBs soaked in water at RT and mixed thoroughly for about 10 minutes to about 1 hour, about 15 minutes to about 45 minutes, or about 30 minutes. High molecular weight HyA with an estimated molecular size range of about 20,000 (about 30 to about 50 ml, or about 40 ml of 1 mg/ml solution) is added and the solution was mixed for additional about 1 to about 4 hours, about 1 to about 3 hours, or about 2 hours in a shaker. The complexed HyA-FMB are rinsed residual non-crosslinked HyA solution and EDC removed. In a non-limiting example, the mean molecular weight of the HyA-FMB polymer is about 20,000 Da.

The rinsed HyA-coated FMBs are re-suspended. In a specific non-limiting example, fibrin microbeads coated with hyaluronic acid (HyA-FMBs) have a density of about 1.1 to about 1.5 g/mL, such as a density of about 1.2 to about 1.4 g/mL, such as a density of about 1.2 to about 1.3 g/mL, such as a density of about 1.2 or about 1.3 g/mL. Fibrin microbeads can have a density of greater than greater than 1.15 g/mL, for example from about 1.1 to about 1.4 g/mL, about 1.2 to about 1.4 g/mL, or about 1.25 to about 1.35 g/mL.

A pharmaceutical composition is also provided that includes a therapeutically effective amount of the chondrocytes derived from a hiPSC. The pharmaceutical composition can also include a hydrogel. The hydrogel can be a gelatin, cellulose and/or collagen-based matrix in combination with bone marrow and/or isolated skeletal stem cells (also referred to as “mesenchymal stem cells”). Thus, the hydrogel can form a biocompatible scaffold for transplantation. The hydrogel can be a photocrosslinked gelatin hydrogel.

Hydrogels can generally absorb fluid and, at equilibrium, typically are composed of 60-90% fluid and only 10-30% polymer. In one embodiment, the water content of hydrogel is about 70-80%. Generally, a hydrogel is biocompatible. A hydrogel can be prepared by crosslinking hydrophilic biopolymers or synthetic polymers (see PCT Application No. WO 2013/040559, incorporated herein by reference). Examples of the hydrogels formed from physical or chemical crosslinking of hydrophilic biopolymers include, but are not limited to, hyaluronans, chitosans, alginates, collagen, dextran, pectin, carrageenan, polylysine, gelatin and/or agarose, (sec.: W. E. Hennink and C. F. van Nostrum, 2002, Adv. Drug Del. Rev. 54, 13-36 and A. S. Hoffman, 2002, Adv. Drug Del. Rev. 43, 3.12). These materials consist of high-molecular weight backbone chains made of linear or branched polysaccharides or polypeptides. The hydrogel can include natural polymers or synthetic (non-natural) polymers.

In one embodiment, hydrogel is a non-biodegradable hydrogel, a natural biodegradable hydrogel, and/or a synthetic biodegradable hydrogel. In certain embodiments, the hydrogel is a self-assembly peptide, a fibrin, an alginate, an agarose, a hyaluronan, a hyaluronic acid, a chitosan, a chondroitin sulfate, a polyethylene oxide (PEO), a poly(ethylene glycol) (PEG), a collagen type I, a collagen type II hydrogel, or combination thereof. In a further embodiment, the hydrogel composition includes a hydrogel selected from the following: self-assembly peptide, fibrin, alginate, agarose, hyaluronan, hyaluronic acid, chitosan, chondroitin sulfate, collagen type I, collagen type II, and combinations thereof. In additional embodiments, the hydrogel includes bioabsorbable materials selected from gelatin, alginic acid, chitin, chitosan, dextran, polyamino acids, polylysine, and copolymers of these materials. In other embodiments, the hydrogel is manufactured from biodegradable materials which degrade in vivo or in vitro, at a sufficiently slow rate to allow the MSC to be therapeutically effective. The hydrogel can be made from alpha hydroxyl polyesters. Exemplary hydrogels are disclosed in U.S. Published Patent Application No. 2007/0098675 and U.S. Published Patent Application No. 2010/0179659, which are both incorporated herein by reference.

Examples of hydrogels based on chemical or physical crosslinking of synthetic polymers include but are not limited to (meth)acrylate-oligolactide-PEO-oligolactide-(meth)acrylate, poly(ethylene glycol) (PEO), poly(propylene glycol) (PPO), PEO-PPO-PEO copolymers (Pluronics), poly(phosphazene), poly(methacrylates), poly(N-vinylpyrrolidone), PL(G)A-PEO-PL(G)A copolymers, polyethylene imine), etc. (see A. S Hoffman, Adv. Drug Del. Rev. 43, 3-12, 2002). Hydrogels can be made degradable in vivo by incorporating PLA, PLGA or PGA polymers. Moreover, hydrogels can be modified with fibronectin, laminin, or vitronectin.

In one embodiment, the hydrogel scaffold includes gelatin. Hydrogels comprised of gelatin have a porous structure, helpful for the support of avian MSCs. Gelatin hydrogels are disclosed, for example, in Lin et al., Tissue Engineering Part A, DOI: 10.1089/ten.tea.2013.0642, 2014, incorporated herein by reference.

In additional embodiments, the hydrogel scaffold includes hyaluronan. The hydrogel scaffold can include gelatin and hyaluronan. In some embodiments, the hydrogel scaffold is prepared by methacrylation of the polymer and then utilizing a photoactivated initiator to start the crosslinking process. Method for producing these gelatin and hyaluronan scaffolds are known in the art and as discussed briefly below.

Altering molecular weights, block structures, degradable linkages, and cross-linking modes can influence strength, elasticity, and degradation properties of the hydrogels (Nguyen and West, 2002, Biomaterials 23(22):4307-14; Ifkovits and Burkick, 2007, Tissue Eng. 13(10):2369-85). Hydrogels can also be modified with functional groups for covalently attaching a variety of proteins (e.g., collagen) or compounds such as therapeutic agents. Therapeutic agents which can be linked to the matrix include, but are not limited to, analgesics, anesthetics, antifungals, antibiotics, anti-inflammatories, antidotes, antihistamines, antimicrobials, antiseptics, anti-arthritics, antivirals, chemotherapeutic agents, a colored or fluorescent imaging agent, corticoids (such as steroids), diagnostic aids, diuretics, enzymes, hormones, minerals, nutritional supplements, a radioisotope, sedatives, sulfonamides, stimulants, tranquilizers, vitamins, and growth factors. The therapeutic agent can also be other small organic molecules, naturally isolated entities or their analogs, organometallic agents, chelated metals or metal salts, peptide-based drugs, or peptidic or non-peptidic receptor targeting or binding agents.

A therapeutic agent can be linked to the hydrogel via a protease sensitive linker or other biodegradable linkage. Molecules which can be incorporated into the hydrogel include, but are not limited to, glycoproteins, fibronectin; peptides and proteins; carbohydrates (both simple and/or complex): proteoglycans; antigens; oligonucleotides (sense and/or antisense DNA and/or RNA): antibodies (for example, to infectious agents, tumors, drugs or hormones); and growth. In one embodiment, the hydrogel includes molecules that aid in the growth and proliferation of a mesenchymal stem cell, when cultured in or on the hydrogel. Non-limiting examples of such molecules can include proteins, peptides, supplements, small molecule inhibitors, glycosaminoglycans. growth factors, nucleic acid sequences. and combinations thereof. These molecules can be a growth factor.

In one non-limiting example, the growth factor is TGFβ. Members of the transforming growth factor (TGF) supergene family, which are multifunctional regulatory proteins, can be utilized. Members of the TGF supergene family include TGF-β, (for example, TGP-β1, TGF-β2, TGF-β3): bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (IGF)), Inhibins (for example, Inhibin A, Inhibin B), growth differentiating factors (for example, GDF-1); and Activins (for example, Activin A, Activin B, Activin AB). In another non-limiting example, the growth factor is a bone morphogenic protein. Growth factors can be isolated from native or natural sources, such as from mammalian cells, or can be prepared synthetically, such as by recombinant DNA techniques or by various chemical processes. In addition, analogs, fragments, or derivatives of these factors can be used, provided that they exhibit at least some of the biological activity of the native molecule. For example, analogs can be prepared by expression of genes altered by site-specific mutagenesis or other genetic engineering techniques.

In certain embodiments, one or more multifunctional cross-linking agents may be utilized as reactive moieties that covalently link biopolymers or synthetic polymers. Such bifunctional cross-linking agents may include glutaraldehyde, epoxides (e.g., bis-oxiranes), oxidized dextran, p-azidobenzoyl hydrazide, N-[a.-maleimidoacetoxy]succinimide ester p-azidophenyl glyoxal monohydrate, bis-[-(4-azidosalicylamido)ethyl]disulfide, bis[sulfosuccinimidyl]suberate, dithiobis[succinimidyl proprionate, disuccinimidyl suberate. 1-ethyl-3-[3-dimethylaminopropyl]carbodiimide hydrochloride (EDC), N-hydroxysuccinimide (NHS) and other bifunctional cross-linking reagents known to those skilled in the art.

Methacrylic anhydride, methacryloyl chloride, and glycidyl methacrylate may be used to add methacrylate groups to one or more monomers of a polymer. Glycidyl methacrylate may be used, for example, for efficiency of reaction.

Polymerizing initiators include electromechanical radiation. Initiation of polymerization may be accomplished by irradiation with visible light, such as 380 to 740 nm, such as about 350 to about 700 nm, such as between about 514 nm and about 365 nm, such as about 380 nm. In some embodiments, the light intensity is about 10 in W/cm³. In some embodiments, polymerization can also include cross-linking with ultraviolet light, such as UVA, UVB, and/or UVC light.

The mechanical properties of a cross-linked polymer matrix, such as a hydrogel may also be related to pore structure. For applications in tissue engineering, scaffolds with different mechanical properties may be desirable depending on the desired clinical application. For example, scaffolds for cartilage tissue engineering in the articular joint must survive higher mechanical stresses than a cartilage tissue engineering system implanted subcutaneously for plastic surgery applications. Thus, hydrogels with mechanical properties that are easily manipulated may be produced.

In one embodiment utilizing a cross-linking agent, polyacrylated materials, such as ethoxylated (20) trimethylpropane triacrylate. can be used as a photo-activated cross-linking agent. Components of an exemplary reaction mixture would include a thermoreversible hydrogel held at 39° C., polyacrylate monomers, such as ethoxylated (20) trimethylpropane triacrylate, a photo-initiator, such as eosin Y, catalytic agents, such as 1-vinyl-2-pyrrolidinone, and triethanolamine. Exposure of this reactive mixture to long-wavelength light (>498 nm) produces a cross-linked hydrogel.

In some embodiments, the hydrogel is a methacrylated gelatin hydrogel, such as a methacrylated hyaluronan (hyaluronic acid) hydrogel. The hydrogel can be a mixture of methacrylated gelatin and methacrylated hyaluronan. The hydrogel can be a gelatin hydrogel, such as a methacrylated gelatin. and/or methacrylated hyaluronan hydrogel that was photocrosslinked with visible light.

A photocrosslinked gelatin can be crosslinked using visible light. Suitable hydrogels are disclosed, for example, in Lin et al., Application of visible light-based projection stereolithography for live cell scaffold fabrication with designed architecture, Biomaterials. 2013 January; 34(2):331-9. doi: 10.1016/j.biomaterials.2012.09.048. Epub 2012 Oct 22, and Lin et al., Cartilage Tissue Engineering Application of Injectable Gelatin Hydrogel with In Situ Visible-Light-Activated Gelation Capability in both Air and Aqueous Solution, Tissue Eng Part A. 2014 Apr 9, which are both incorporated herein by reference.

A cross-linked hydrogel matrix can be further stabilized and enhanced through the addition of one or more enhancing agents. Enhancing agents include any compound added to the hydrogel matrix, in addition to the high molecular weight components, that enhances the hydrogel matrix by providing further stability or functional advantages. These include, for example, polar amino acids, amino acid analogues, amino acid derivatives, intact collagen, and divalent cation chelators, such as ethylenediaminetetraacetic acid (EDTA) or salts thereof. Polar amino acids are intended to include tyrosine, cysteine, serine, threonine, asparagine, glutamine, aspartic acid, glutamic acid, arginine, lysine. and histidine. In specific non-limiting examples, one or more of L-cysteine, L-glutamic acid, L-lysine, and/or L-arginine is utilized. An enhancing agent can be added to the matrix composition before or during the crosslinking of the high molecular weight components.

Methods for Producing Chondrospheroids and Chondrocytes Derived from hiPSC

### A. Induced Pluripotent Stem Cells

The disclosed non-natural chondrocytes are produced from human iPSC (hiPSC). The induction of pluripotency was originally achieved in 2006 using mouse cells (originally described by Yamanaka et al.) and in 2007 using human cells by reprogramming of somatic cells via the introduction of transcription factors that are linked to pluripotency. Pluripotent stem cells can be maintained in an undifferentiated state and are capable of differentiating into almost any cell type. The use of iPSCs circumvents most of the ethical and practical problems associated with large-scale clinical use of ESCs, and patients with iPSC-derived autologous transplants may not require lifelong immunosuppressive treatments to prevent graft rejection.

With the exception of germ cells, any human cell can be used as a starting point for iPSCs. For example, cell types could be keratinocytes, fibroblasts, hematopoietic cells, mesenchymal cells, liver cells, or stomach cells. The cells can be a multipotent cells, such as but not limited to a hematopoietic stem cell, such as, but no limited to, CD34+ cells. T cells may also be used as a source of somatic cells for reprogramming (U.S. Pat. No. 8,741,648). The human cell can be from the subject that will be treated by the disclosed methods. Thus, the human cell can be autologous. However, the human cell can be from any subject. There is no limitation on the degree of cell differentiation or the age of the subject from which cells are collected; even undifferentiated progenitor cells (including somatic stem cells) and finally differentiated mature cells can be used as sources of somatic cells in the methods disclosed herein. iPSCs can be grown under conditions that are known to differentiate human ES cells into specific cell types, and express human ES cell markers including: SSEA-1, SSEA-3, SSEA-4, TRA-1-60, and TRA-1-81.

Somatic cells and pluripotent stem cells, such as CD34+ cells, can be reprogrammed to produce induced pluripotent stem cells (iPSCs) using methods known to one of skill in the art. One of skill in the art can readily produce induced pluripotent stem cells, see for example, Published U.S. Patent Application No. 20090246875, Published U.S. Patent Application No. 2010/0210014; Published U.S. Patent Application No. 20120276636; U.S. Pat. No. 8,058,065: U.S. Pat. Nos. 8,129,187; 8,278,620; PCT Publication NO. WO 2007/069666 A1, and U.S. Pat. No. 8,268,620, which are incorporated herein by reference in its entirety. Generally, nuclear reprogramming factors are used to produce pluripotent stem cells from a somatic cell. In some aspects, at least three, or at least four, of Klf4, c-Myc, Oct3/4, Sox2, Nanog, and Lin28 are utilized. In other aspects, Oct3/4, Sox2, c-Myc and Klf4 are utilized.

The cells are treated with a nuclear reprogramming substance, which is generally one or more factor(s) capable of inducing an iPSC from a somatic cell or a nucleic acid that encodes these substances (including forms integrated in a vector). The nuclear reprogramming substances generally include at least Oct3/4, Klf4 and Sox2 or nucleic acids that encode these molecules. A functional inhibitor of p53, L-myc or a nucleic acid that encodes L-myc, and Lin28 or Lin28b or a nucleic acid that encodes Lin28 or Lin28b, can be utilized as additional nuclear reprogramming substances. Nanog can also be utilized for nuclear reprogramming. As disclosed in published U.S. Patent Application No. 20120196360, exemplary reprogramming factors for the production of iPSCs include (1) Oct3/4, Klf4, Sox2, L-Myc (Sox2 can be replaced with Sox1, Sox3, Sox15, Sox17 or Sox18; Klf4 is replaceable with Klf1, Klf2 or Klf5); (2) Oct3/4, Klf4, Sox2, L-Myc, TERT, SV40 Large T antigen (SV40LT); (3) Oct3/4, Klf4, Sox2, L-Myc, TERT, human papilloma virus (HPV)16 E6; (4) Oct3/4, Klf4, Sox2, L-Myc, TERT, HPV16 E7 (5) Oct3/4, Klf4, Sox2, L-Myc, TERT, HPV16 E6, HPV16 E7; (6) Oct3/4, Klf4, Sox2, L-Myc, TERT, Bmil; (7) Oct3/4, Klf4, Sox2, L-Myc, Lin28; (8) Oct3/4, Klf4, Sox2, L-Myc, Lin28, SV40LT; (9) Oct3/4, Klf4, Sox2, L-Myc, Lin28, TERT, SV40LT; (10) Oct3/4, Klf4, Sox2, L-Myc, SV40LT; (11) Oct3/4, Esrrb, Sox2, L-Myc (Esrrb is replaceable with Esrrg); (12) Oct3/4, Klf4, Sox2; (13) Oct3/4, Klf4, Sox2, TERT, SV40LT; (14) Oct3/4, Klf4, Sox2, TERT, HP VI 6 E6; (15) Oct3/4, Klf4, Sox2, TERT, HPV16 E7; (16) Oct3/4, Klf4, Sox2, TERT, HPV16 E6, HPV16 E7; (17) Oct3/4, Klf4, Sox2, TERT, Bmil; (18) Oct3/4, Klf4, Sox2, Lin28 (19) Oct3/4, Klf4, Sox2, Lin28, SV40LT; (20) Oct3/4, Klf4, Sox2, Lin28, TERT, SV40LT; (21) Oct3/4, Klf4, Sox2, SV40LT; or (22) Oct3/4, Esrrb, Sox2 (Esrrb is replaceable with Esrrg). In one non-limiting example, Oct3/4, Klf4, Sox2, and c-Myc are utilized. In other aspects, Oct4, Nanog, and Sox2 are utilized, see for example, U.S. Pat. No. 7,682,828, which is incorporated herein by reference in its entirety. These factors include, but are not limited to, Oct3/4, Klf4 and Sox2. In other examples, the factors include, but are not limited to Oct 3/4, Klf4 and Myc. In some non-limiting examples, Oct3/4, Klf4, c-Myc, and Sox2 are utilized. In other non-limiting examples, Oct3/4, Klf4, Sox2 and Sal 4 are utilized. Factors like Nanog, Lin28, Klf4, or c-Myc can increase reprogramming efficiency and can be expressed from several different expression vectors. For example, an integrating vector such as the EBV element-based system can be used (U.S. Pat. No. 8,546,140). In a further aspect, reprogramming proteins could be introduced directly into somatic cells by protein transduction. Reprogramming may further comprise contacting the cells with one or more signaling receptors including glycogen synthase kinase 3 (GSK-3) inhibitor, a mitogen-activated protein kinase (MEK) inhibitor, a transforming growth factor beta (TGF-β) receptor inhibitor or signaling inhibitor, leukemia inhibitory factor (LIF), a p53 inhibitor, an NF-kappa B inhibitor, or a combination thereof. Those regulators may include small molecules, inhibitory nucleotides, expression cassettes, or protein factors. It is anticipated that virtually any iPS cells or cell lines may be used.

Mouse and human cDNA sequences of these nuclear reprogramming substances are available with reference to the NCBI accession numbers recited in WO 2007/069666, which is incorporated herein by reference in its entirety. Methods for introducing one or more reprogramming substances, or nucleic acids encoding these reprogramming substances, are known i, and disclosed for example, in U.S. Patent Application No. 2012/0196360 and U.S. Pat. No. 8,071,369, which both are incorporated herein by reference in its entirety.

Once derived, iPSCs can be cultured in a medium sufficient to maintain pluripotency. The iPSCs may be used with various media and techniques developed to culture pluripotent stem cells, more specifically, embryonic stem cells, as described in U.S. Pat. No. 7,442,548 and U.S. Patent Pub. No. 2003/0211603. In the case of mouse cells, the culture is carried out with the addition of Leukemia Inhibitory Factor (LIF) as a differentiation suppression factor to an ordinary medium. In the case of human cells, it is desirable that basic fibroblast growth factor (bFGF) be added in place of LIF. Other methods for the culture and maintenance of iPSCs, may be used.

In certain aspects, undefined conditions may be used; for example, pluripotent cells may be cultured on fibroblast feeder cells or a medium that has been exposed to fibroblast feeder cells in order to maintain the stem cells in an undifferentiated state. In some aspects, the cell is cultured in the co-presence of mouse embryonic fibroblasts treated with radiation or an antibiotic to terminate the cell division, as feeder cells. Alternatively, pluripotent cells may be cultured and maintained in an essentially undifferentiated state using a defined, feeder-independent culture system, such as a TESR™ medium or E8™ medium.

In some aspects, the hiPSCs can be modified, such as to express an exogenous gene, increase expression of an endogenous gene, increase copy number of a gene, to correct a gene mutation, or to silence the expression of a mutant gene. In some specific non-limiting examples, a mutation or a deletion in an endogenous gene is corrected.

Methods for performing gene editing in iPSCs are disclosed, for example, in Hockenmeyer and Jaenisch, “Induced Pluripotent Stem Cell Meets Genome Editing,” Cell Stem Cell 18: 573-586, 2016, incorporated herein by reference in its entirety. Any of the methods disclosed therein are of use. The method can include the use of a viral vector, such as an adeno-associated viral vector or a lentiviral vector ending a transgene of interest. The method can include the use of CRISPR/Cas9, TALEN nuclease, Zinc-finger nuclease, lentiviral mediated correction, adeno-associated virus mediated correction, shRNA, siRNA, or F-prime editing.

In some aspects, the hiPSC can be modified to express exogenous nucleic acids, such as to include a tissue specific promoter operably linked to a promoter and a nucleic acid sequence encoding a first marker. Suitable promoters include, but are not limited to, any promoter expressed in chondrocytes including a collagen promoter. The construct can also include other elements, such as a ribosome binding site for translational initiation (internal ribosomal binding sequences), and a transcription/translation terminator. Generally, it is advantageous to transfect cells with the construct. Suitable vectors for stable transfection include, but are not limited to retroviral vectors, lentiviral vectors and Sendai virus.

Plasmids can achieve regulated high copy number and are compatible with use in mammalian cells, including human cells. In some examples, plasmids, they are suitable for maintenance and fermentation in E. coli, so that large amounts of DNA can be produced and purified. Plasmids can be safe and suitable for use in human patients and animals. High copy number plasmids can be selected for and stably maintained relatively easily during bacterial fermentation. Elements such as selectable markers and other coding sequences can be included in a plasmid. In some aspects plasmids that encode a marker include: (1) a high copy number replication origin, (2) a selectable marker, such as, but not limited to, the neo gene for antibiotic selection with kanamycin, (3) transcription termination sequences, (4) a multicloning site for incorporation of various nucleic acid cassettes, and (5) a nucleic acid sequence encoding a marker operably linked to the promoter. There are numerous plasmid vectors that are known in the art for inducing a nucleic acid encoding a protein, such as the vectors disclosed in U.S. Pat. Nos. 6,103,470; 7,598,364; 7,989,425; and 6,416,998, which are incorporated herein by reference in their entireties.

A viral gene delivery system can be an RNA-based or DNA-based viral vector. An episomal gene delivery system can be a plasmid, an Epstein-Barr virus (EBV)-based episomal vector, a yeast-based vector, an adenovirus-based vector, a simian virus 40 (SV40)-based episomal vector, a bovine papilloma virus (BPV)-based vector, or a lentiviral vector.

In some aspects, the cells are transfected with a nucleic acid molecule encoding a marker. Markers include, but are not limited to, fluorescence proteins (for example, green fluorescent protein or red fluorescent protein), enzymes (for example, horse radish peroxidase or alkaline phosphatase or firefly/renilla luciferase or nanoluc), or other proteins. A marker may be a protein (including secreted, cell surface, or internal proteins; either synthesized or taken up by the cell); a nucleic acid (such as an mRNA, or enzymatically active nucleic acid molecule) or a polysaccharide. Included are determinants of any such cell components that are detectable by antibody, lectin, probe or nucleic acid amplification reaction that are specific for the marker of the cell type of interest. The markers can also be identified by a biochemical or enzyme assay or biological response that depends on the function of the gene product.

### 1. MHC Haplotype Matching

Major Histocompatibility Complex is the main cause of immune-rejection of allogeneic organ transplants. There are three major class I MHC haplotypes (A, B, and C) and three major MHC class II haplotypes (DR, DP, and DQ). The HLA loci are highly polymorphic and are distributed over 4 Mb on chromosome 6. The ability to haplotype the HLA genes within the region is clinically important since this region is associated with autoimmune and infectious diseases and the compatibility of HLA haplotypes between donor and recipient can influence the clinical outcomes of transplantation. HLAs corresponding to MHC class I present peptides from inside the cell and HLAs corresponding to MHC class II present antigens from outside of the cell to T-lymphocytes. Incompatibility of MHC haplotypes between the graft and the host triggers an immune response against the graft and leads to its rejection. Thus, a subject can be treated with an immunosuppressant to prevent rejection. HLA-matched stem cell lines may overcome the risk of immune rejection.

Because of the importance of HLA in transplantation, the HLA loci are usually typed by serology and PCR for identifying favorable donor-recipient pairs. Serological detection of HLA class I and II antigens can be accomplished using a complement mediated lymphocytotoxicity test with purified T or B lymphocytes. This procedure is predominantly used for matching HLA-A and -B loci. Molecular-based tissue typing can often be more accurate than serologic testing. Low resolution molecular methods such as SSOP (sequence specific oligonucleotide probes) methods, in which PCR products are tested against a series of oligonucleotide probes, can be used to identify HLA antigens, and currently these methods are the most common methods used for Class II-HLA typing. High resolution techniques such as SSP (sequence specific primer) methods which utilize allele specific primers for PCR amplification can identify specific MHC alleles.

MHC compatibility between a donor and a recipient increases significantly if the donor cells are HLA homozygous, i.e., contain identical alleles for each antigen-presenting protein. Most individuals are heterozygous for MHC class I and II genes, but certain individuals are homozygous for these genes. These homozygous individuals can serve as super donors and grafts generated from their cells can be transplanted in all individuals that are either homozygous or heterozygous for that haplotype. Furthermore, if homozygous donor cells have a haplotype found in high frequency in a population, these cells may have application in transplantation therapies for a large number of individuals.

Accordingly, iPSCs can be produced from cells of the subject to be treated, or another subject with the same or substantially the same HLA type as that of the subject. In one case, the major HLAs (e.g., the three major loci of HLA-A, HLA-B and HLA-DR) of the donor are identical to the major HLAs of the recipient. In some cases, the somatic cell donor may be a super donor; thus, iPSCs derived from a MHC homozygous super donor may be used to generate chondrocytes. Thus, the iPSCs derived from a super donor may be transplanted in subjects that are either homozygous or heterozygous for that haplotype. For example, the iPSCs can be homozygous at two HLA alleles such as HLA-A and HLA-B. As such, iPSCs produced from super donors can be used in the methods disclosed herein, to produce chondrocytes that can potentially “match” a large number of potential recipients.

### 2. Episomal Vectors

In certain aspects, reprogramming factors are expressed from expression cassettes comprised in one or more exogenous episomal genetic elements (see U.S. Patent Publication 2010/0003757, incorporated herein by reference in its entirety). Thus, iPSCs can be essentially free of exogenous genetic elements, such as from retroviral or lentiviral vector elements. These iPSCs are prepared by the use of extra-chromosomally replicating vectors (i.e., episomal vectors), which are vectors capable of replicating episomally to make iPSCs essentially free of exogenous vector or viral elements (see U.S. Pat. No. 8,546,140, incorporated herein by reference in its entirety). A number of DNA viruses, such as adenoviruses, simian virus 40 (SV40) or bovine papilloma virus (BPV), or budding yeast ARS (Autonomously Replicating Sequences)-containing plasmids replicate extra-chromosomally or episomally in mammalian cells. These episomal plasmids are intrinsically free from all these disadvantages associated with integrating vectors. For example, a lymphotrophic herpes virus-based including or Epstein Barr Virus (EBV) as defined above may replicate extra-chromosomally and help deliver reprogramming genes to somatic cells. Useful EBV elements are OriP and EBNA-1, or their variants or functional equivalents. One advantage of episomal vectors is that the exogenous elements will be lost with time after being introduced into cells, leading to self-sustained iPSCs essentially free of these elements.

Other extra-chromosomal vectors include other lymphotrophic herpes virus-based vectors. Lymphotrophic herpes virus is a herpes virus that replicates in a lymphoblast (e.g., a human B lymphoblast) and becomes a plasmid for a part of its natural life-cycle. Herpes simplex virus (HSV) is not a “lymphotrophic” herpes virus. Exemplary lymphotrophic herpes viruses include, but are not limited to EBV, Kaposi's sarcoma herpes virus (KSHV); herpes virus saimiri (HS) and Marek's disease virus (MDV). Additional sources of episome-based vectors are contemplated, such as yeast ARS, adenovirus, SV40, or BPV.

### B. Culture Conditions

Methods are disclosed herein for producing chondrocytes derived from a hiPSC. In some aspects, the methods include treating sclerotome cells with and effective amount of a transforming growth factor (TGF)β, a bone morphogenic protein (BMP), and a bone morphogenic protein receptor (BMPR)1A/B agonist to produce a chondrospheroid. In further aspects, these methods include treating the chondrospheroid with an effective amount of a TGFβ, a BMP, and/or a bone morphogenic protein receptor BMPR1A/B agonist, thereby producing the chondrospheroid. These steps, and additional steps, are discussed in further detail below.

### 1. Production of Sclerotome

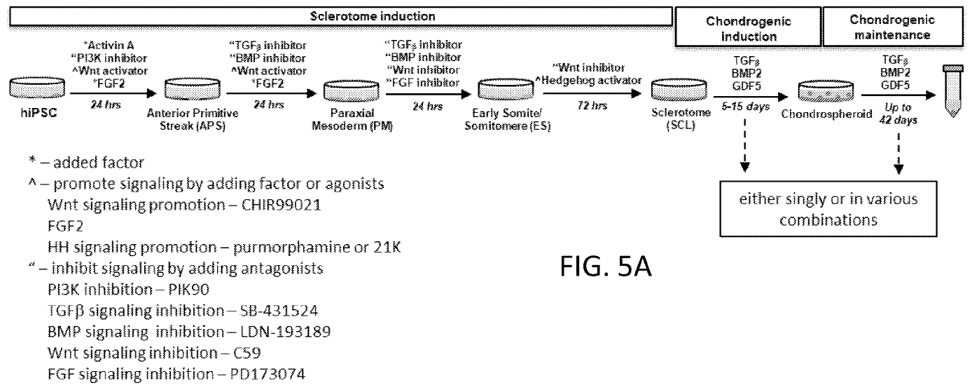

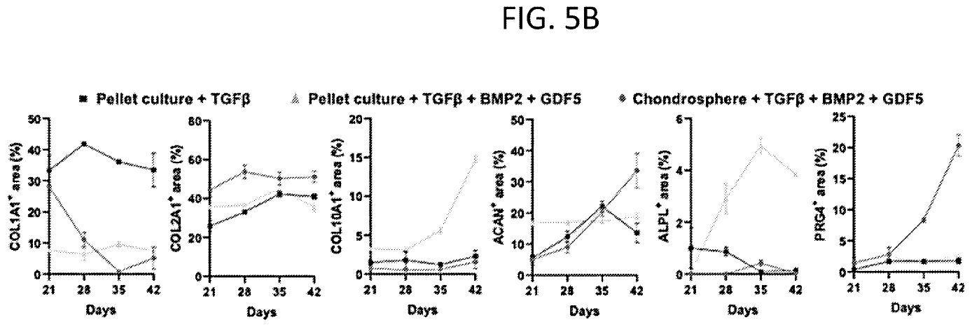

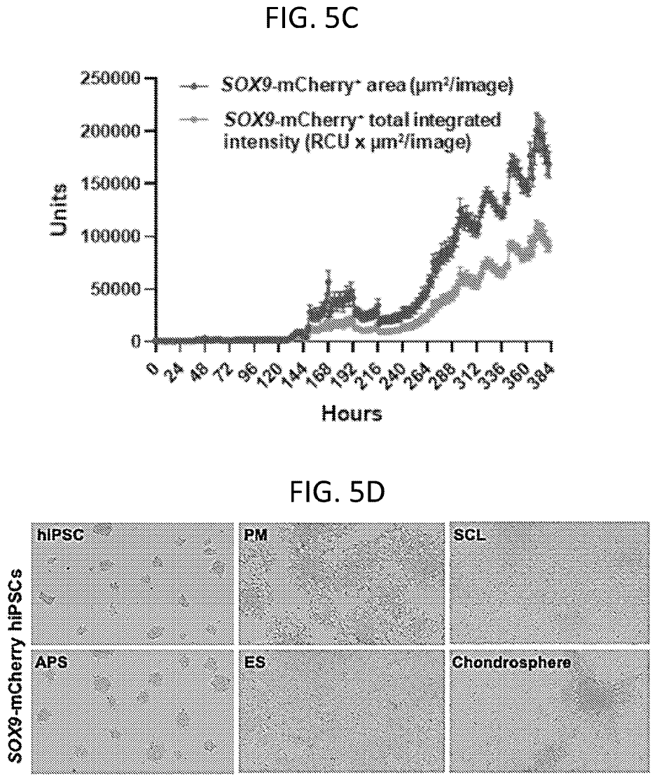

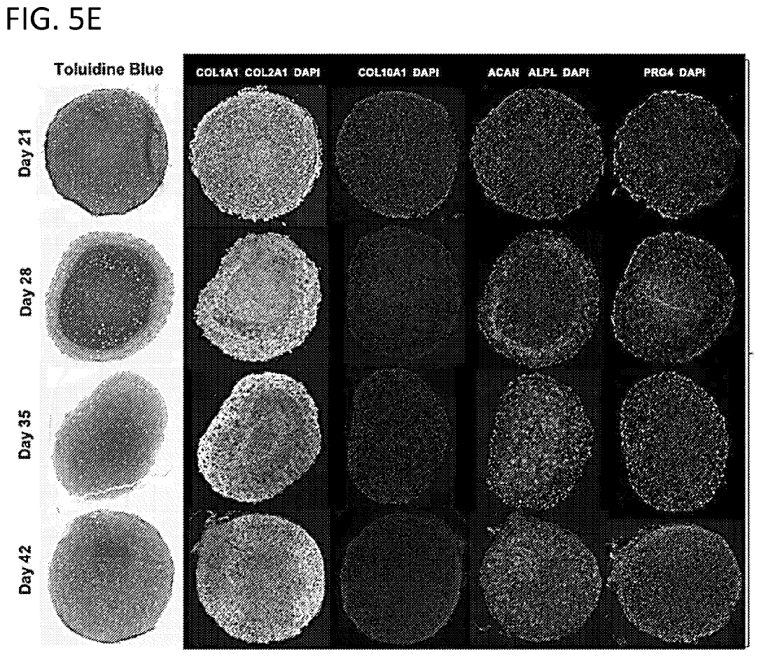

Methods for the production of sclerotome are disclosed, for example, in Loh et al., Cell, 166(2), 451-467, doi.org/10.1016/j.cell.2016.06.011, 2016, and U.S. Pat. No. 10,787,640 (“Producing mesodermal cell types and methods of using the same,” Loh et al., issued Sep. 29, 2020), both incorporated herein by reference. In some aspects, the method includes a) differentiating human induced pluripotent stem cells into anterior primitive streak cells; b) differentiating the anterior primitive streak cells to paraxial mesoderm cells; c) differentiating the paraxial mesoderm cells into early somite cells; and d) differentiating the early somite cells into the sclerotome cells. A schematic diagram of these steps is shown in FIG. 5A.

In some aspects, the method includes differentiating hiPSC into anterior primitive streak cells by treating the human induced pluripotent cells with an effective amount of Activin A, an effective amount of Phosphoinositide 3-kinase (PI3K) inhibitor, a Wnt activator, and FGF2. The hiPSC can be treated for about 12 to about 48 hours, such as about 24 to about 48 hours. In a non-limiting example, the hiPSC are treated for about 24 hours.

In a specific non-limiting example, the Wnt activator is CHIR99021. In more non-limiting examples, the PI3K inhibitor is PIK90. In another specific non-limiting example, iPSC are treated with an effective amount of Activin A, PIK90, CHIR99021, and FGF-2 to produce anterior primitive streak cells. Anterior primitive streak cells have high expression of MIXL1, BRACHYURY, GSC, EOMES, and FOXA2 and reduced expression of posterior primitive streak markers (MESP1, MESP2, FOXF1) compared with iPSCs.

In further aspects, the method includes differentiating the anterior primitive streak cells to paraxial mesoderm cells, by treating the anterior primitive streak cells with an effective amount of a TGFβ inhibitor, a BMP inhibitor, a Wnt activator and FGF2. The anterior primitive streak cells can be treated for about 12 to about 48 hours, such as about 24 to about 48 hours. In a non-limiting example, the anterior primitive streak cells are treated for about 24 hours.

In a specific non-limiting examples, the TGFβ inhibitor is SB-431524. In more non-limiting examples, the BMP inhibitor is LDN-193189. In further non-limiting examples, the Wnt activator is CHIR99021. The method can include treating the anterior primitive streak cells with an effective amount of SB-431524, LDN-193189, CHIR99021, and FGF2.

In more aspects, the method includes differentiating the paraxial mesoderm cells into early somite cells using an effective amount of a TGFβ inhibitor, a BMP inhibitor, a Wnt inhibitor, and a FGF inhibitor. In some examples, the FGF inhibitor is an ERK inhibitor. The paraxial mesoderm cells can be treated for about 12 to about 48 hours, such as about 24 to about 48 hours. In a non-limiting example, the paraxial mesoderm cells are treated for about 24 hours.

In a further specific non-limiting examples, the TGFβ inhibitor is SB-431524. In more non-limiting examples, the BMP inhibitor is LDN-193189. In further non-limiting examples, the Wnt inhibitor is C59. In more non-limiting examples, the FGF inhibitor is PD173074. Thus, the method can include treating the paraxial mesoderm cells with an effective amount of SB-431524, LDN-193189, C59, and PD173074 to produce the early somite cells.

In additional aspects, the method includes differentiating the early somite cells into sclerotome using an effective amount of a Wnt inhibitor and a Hedgehog activator. The early somite cells can be treated for about 48 hours to about 96 hours, such as about 48 hours to about 72 hours, or about 72 hours to about 96 hours. In a non-limiting example, the early somite cells are treated for about 72 hours.

In specific non-limiting examples, the Wnt inhibitor is 4-(2-Methyl-4-pyridinyl)-N-[4-(3-pyridinyl)phenyl]benzeneacetamide (C59). In more non-limiting examples, the Hedgehog activator is purmorphamine or 21K. In further non-limiting examples, the Wnt inhibitor is C59, and the Hedgehog activator is purmorphamine or 3-chloro-4,7-difluoro-N-(4-methoxy-3-(pyridin-4-yl)benzyl)-N-((1r,4r)-4-(methylamino)cyclohexyl)benzo[b]-thiophene-2-carboxamide (21K).

One of skill in the art can readily determine an effective amount of an inhibitor or activator. Exemplary effective amounts are disclosed, for example, in U.S. Pat. No. 10,787,640, and the below examples.

### 2. Production of Chondrospheroids

In some aspects, the methods include producing chondrospheroids by treating sclerotome cells, produced from iPSC (see above), with an effective amount of a TGFβ, a BMP, and/or a BMPR1A/B agonist. The method can include treating a sclerotome cells with an effective amount of two of a TGFs, a BMP, and a BMPR1A/B agonist. In one example, the sclerotome cells can be treated with TGFβ and a BMP. In another example, the sclerotome cells can be treated with TGFβ and a BMPR1A/B agonist. In a further example, the sclerotome cells can be treated with a BMP and a BMPR1A/B agonist. In yet other examples, the method can include treating the sclerotome cells produced from iPSC with an effective amount of two of a TGFβ, a BMP, and a BMPR1A/B agonist. In yet other aspects, the method includes treating the sclerotome cells with an effective amount of BMP-2, GDF-5 and TGFβ1.

The sclerotome cells can be treated for about 5 to about 15 days, such as about 7 to about 12 days, such as about 10 days. The sclerotome cells can be treated for about 5, 6, 7, 8, 9, or 10 days. In a non-limiting example, the sclerotome cells are treated for about 10 days.

In some aspects, the TGFβ is TGFβ1 or TGFβ3. In more aspects, the BMP is BMP-2 or BMP-4. In further aspects, the BMPR1A/B agonist is growth differentiation factor (GDF)-5, BMP-4, BMP-5, BMP-6, BMP-7, BMP-8A, BMP-8B, BMP-10, BMP-11, GDF-6, GDF-7, or anti-Müllerian hormone (AMH).

In some aspects, the TGFβ is TGFβ1. In more aspects, the BMP is BMP2. In further aspects, the BMPR1A/B agonist is GDF5. In yet other aspects, the method includes treating sclerotome cells, produced from iPSC (see above), with an effective amount of BMP-2, GDF-5 and TGFβ1. In further aspects, the method includes treating sclerotome cells produced from hiPSC (see above), with an effective amount of BMP-2, GDF-5 and TGFβ1 for about 10 days to produce a chondrospheroid.

In some aspects, the method includes the use of about 1 to about 25 ng/mL of TGFβ1, such as about 5 to about 20 ng/mL of TGFβ1, such as about 7 to about 15 ng/mL of TGFβ1. In one example, the method includes the use of about 10 ng/mL TGFβ1.

In some aspects, the method includes the use of about 1 to about 25 ng/mL of BMP-2, such as about 5 to about 20 ng/mL of BMP-2, such as about 7 to about 15 ng/mL of BMP-2. In one example, the method includes the use of about 10 ng/mL BMP-2.

In some aspects, the method includes the use of about 1 to about 25 ng/mL of GDF-5, such as about 5 to about 20 ng/mL of GDF-5, such as about 7 to about 15 ng/mL of GDF-5. In one example, the method includes the use of about 10 ng/mL GDF-5.

In more aspects, the method includes the use of about 10 ng/mL TGFβ1, about 10 ng/mL BMP-2, and about 10 ng/mL GDF-5.

### 3. Production of Chondrocytes

In some aspects, a chondrospheroid is transferred to suspension culture and treated with an effective amount of TGFβ, a BMP, and/or a BMPR1A/B agonist, thereby producing the chondrocyte.

In some aspects, the chondrocytes are in the form of an aggregate in the suspension culture. In more aspects, the method includes digesting the aggregate into single cells. The aggregate can be digested with an effective amount of collagenase, dispase, liberase, and/or trypsin. Exemplary methods are provided in the examples section. The digested chondrospheroid is then transferred to suspension culture and treated with an effective amount of TGFβ, a BMP, and/or a BMPR1A/B agonist, thereby producing the chondrocyte.

The method can include treating a chondrospheroid, digested or undigested, with an effective amount of two of a TGFβ, a BMP, and a BMPR1A/B agonist. In other aspects, the chondrospheroid cells can be treated with a TGFβ and a BMP. In another example, the chondrospheroid cells can be treated with TGFβ and a BMPR1A/B agonist. In a further example, the chondrospheroid cells can be treated with a BMP and a BMPR1A/B agonist. In yet other examples, the method can include treating chondrospheroid cells produced from iPSC with an effective amount of two of a TGFβ, a BMP, and a BMPR1A/B agonist. In yet other aspects, the method includes treating the chondrospheroid with an effective amount of BMP-2, GDF-5 and TGFβ1. The chondrospheroid can be digested or undigested, in the form of the aggregate.

In some aspects, the TGFβ is TGFβ1. In more aspects, the BMP is BMP2. In further aspects, the BMPR1A/B agonist is GDF5. In some aspects, the TGFβ is TGFβ1. In more aspects, the BMP is BMP-2. In further aspects, the BMPR1A/B agonist is GDF5.

In further aspects, the method includes treating the chondrospheroid (digested or undigested) with an effective amount of BMP-2, GDF-5 and/or TGFβ1 for about 42 days, such as for 40-44 days. The treatment can be for 20, 30, 35, 40, or up to 42 days. The treatment can be for about 20 days to about 60 days, such as 20 days to 42 days, such as 20, 21, 22, 23, 24, 25, 26, 27, 28, 29, 30, 31, 32, 33, 34, 35, 36, 37, 38, 39, 40, 41 or 42 days, or any range in between these values. The treatment can be for more than 42 days. The treatment can be for about 42, 43, 44, 45, 46, 47, 48, 49, 50, 51, 52, 53, 54, 55, 56, 57, 58, 59 or 60 days. The treatment can be for about 42 days to about 50 days.

In a specific non-limiting example, the method includes treating an undigested chondrospheroid with an effective amount of BMP-2, GDF-5 and/or TGFβ31 for about 1 day in a suspension culture to produce chondrocytes, for up to 42 days.

In some aspects, the method includes the use of about 1 to about 25 ng/mL of TGFβ1, such as about 5 to about 20 ng/mL of TGFβ1, such as about 7 to about 15 ng/mL of TGFβ1. In one example, the method includes the use of about 10 ng/mL TGFβ1.

In some aspects, the method includes the use of about 1 to about 25 ng/mL of BMP-2, such as about 5 to about 20 ng/mL of BMP-2, such as about 7 to about 15 ng/mL of BMP-2. In one example, the method includes the use of about 10 ng/mL BMP-2.

In some aspects, the method includes the use of about 1 to about 25 ng/mL of GDF-5, such as about 5 to about 20 ng/mL of GDF-5, such as about 7 to about 15 ng/mL of GDF-5. In one example, the method includes the use of about 10 ng/mL GDF-5.

In more aspects, the method includes the use of about 10 ng/mL TGFβ1, about 10 ng/mL BMP-2, and about 10 ng/mL GDF-5.

4. Molecules of Use in Differentiation of iPSC to Chondrocytes

Disclosed below are molecules that are of use in preparing chondrocytes. These molecules can be included in the media used in the above disclosed methods. Suitable activators and inhibitors include small molecules, peptides, chemical compounds, and nucleic acid molecules. Exemplary molecules of use in the disclosed methods are provided below.

a. WNT Pathway Inhibitors

Wnt is a family of highly conserved secreted signaling molecules that regulate cell-to-cell interactions and are related to the Drosophila segment polarity gene, wingless. In humans, the Wnt family of genes encodes 38 to 43 kDa cysteine rich glycoproteins. The Wnt proteins have a hydrophobic signal sequence, a conserved asparagine-linked oligosaccharide consensus sequence (see e.g., Shimizu et al., Cell Growth Differ 8: 1349-1358 (1997)) and 22 conserved cysteine residues. Because of their ability to promote stabilization of cytoplasmic beta-catenin, Wnt proteins can act as transcriptional activators and inhibit apoptosis. Overexpression of particular Wnt proteins has been shown to be associated with certain cancers.

A Wnt inhibitor herein refers to Wnt inhibitors in general. Thus, a Wnt inhibitor refers to any inhibitor of a member of the Wnt family proteins including Wnt1, Wnt2, Wnt2b, Wnt3, Wnt4, Wnt5A. Wnt6, Wnt7A, Wnt7B, Wnt8A, Wnt9A, Wnt10a, Wnt11, and Wnt16. Certain aspects of the present methods concern a WNT inhibitor in the differentiation medium. Examples of suitable Wnt inhibitors, already known in the art, include N-(2-Aminoethyl)-5-chloroisoquinoline-8-sulphonamide dihydrochloride (CKI-7), N-(6-Methyl-2-benzothiazolyl)-2-[(3,4,6,7-tetrahydro-4-oxo-3-phenylthieno[3,2-d]pyrimidin-2-yl)thio]-acetamide (IWP2), N-(6-Methyl-2-benzothiazolyl)-2-[(3,4,6,7-tetrahydro-3-(2-methoxyphenyl)-4-oxothieno[3,2-d]pyrimidin-2-yl)thio]-acetamide (IWP4), 2-Phenoxybenzoic acid-[(5-methyl-2-furanyl)methylene]hydrazide (PNU 74654) 2,4-diamino-quinazoline, quercetin, 3,5,7,8-Tetrahydro-2-[4-(trifluoromethyl)phenyl]-4H-thiopyrano[4,3-d]pyrimidin-4-one (XAV939), 2,5-Dichloro-N-(2-methyl-4-nitrophenyl)benzenesulfonamide (FH 535), N-[4-[2-Ethyl-4-(3-methylphenyl)-5-thiazolyl]-2-pyridinyl]benzamide (TAK 715), Dickkopf-related protein one (DKK1), and Secreted frizzled-related protein (SFRP1) 1. In addition, inhibitors of Wnt can include antibodies to, dominant negative variants of, and siRNA and antisense nucleic acids that suppress expression of Wnt. Inhibition of Wnt can also be achieved using RNA-mediated interference (RNAi). The Wnt inhibitor can be C₂₅H₂₁N₃O, which is 4-(2-Methyl-4-pyridinyl)-N-[4-(3-pyridinyl)phenyl]benzeneacetamide (C59).

b. BMP Pathway Inhibitors

Bone morphogenic proteins (BMPs) are multi-functional growth factors that belong to the transforming growth factor beta (TGFβ) superfamily. BMPs are considered to constitute a group of pivotal morphogenetic signals, orchestrating architecture through the body. The important functioning of BMP signals in physiology is emphasized by the multitude of roles for dysregulated BMP signaling in pathological processes.

BMP pathway inhibitors may include inhibitors of BMP signaling in general or inhibitors specific for BMP1, BMP2, BMP3, BMP4, BMP5, BMP6, BMP7, BMP8a, BMP8b, BMP10 or BMP15. Exemplary BMP inhibitors include 4-(6-(4-(piperazin-1-yl)phenyl)pyrazolo[1,5-a]pyrimidin-3-yl)quinoline hydrochloride (LDN193189), 6-[4-[2-(1-Piperidinyl)ethoxy]phenyl]-3-(4-pyridinyl)-pyrazolo[1,5-a]pyrimidine dihydrochloride (Dorsomorphin), 4-[6-[4-(1-Methylethoxy)phenyl]pyrazolo[1,5-a]pyrimidin-3-yl]-quinoline (DMH1), 4-[6-[4-[2-(4-Morpholinyl)ethoxy]phenyl]pyrazolo[1,5-a]pyrimidin-3-yl]quinoline (DMH-2), and 5-[6-(4-Methoxyphenyl)pyrazolo[1,5-a]pyrimidin-3-yl]quinoline (ML 347).

c. TGFβ Pathway Inhibitors

Transforming growth factor beta (TGFβ) is a secreted protein that controls proliferation, cellular differentiation, and other functions in most cells. It is a type of cytokine which plays a role in immunity, cancer, bronchial asthma, lung fibrosis, heart disease, diabetes, and multiple sclerosis. TGF-β exists in at least three isoforms called TGF-β1, TGF-β2 and TGF-β3. The TGF-β family is part of a superfamily of proteins known as the transforming growth factor beta superfamily, which includes inhibin, activin, anti-mullerian hormone, bone morphogenetic protein, decapentaplegic and Vg-1.

TGFβ pathway inhibitors may include any inhibitors of TGFβ signaling in general. For example, the TGFβ pathway inhibitor is 4-[4-(1,3-benzodioxol-5-yl)-5-(2-pyridinyl)-1H-imidazol-2-yl]benzamide (SB431542), 6-[2-(1,1-Dimethylethyl)-5-(6-methyl-2-pyridinyl)-1H-imidazol-4-yl]quinoxaline (SB525334), 2-(5-Benzo[1,3]dioxol-5-yl-2-tert-butyl-3H-imidazol-4-yl)-6-methylpyridine hydrochloride hydrate (SB-505124), 4-(5-Benzol[1,3]dioxol-5-yl-4-pyridin-2-yl-1H-imidazol-2-yl)-benzamide hydrate, 4-[4-(1,3-Benzodioxol-5-yl)-5-(2-pyridinyl)-1H-imidazol-2-yl]-benzamide hydrate, left-right determination factor (Lefty), 3-(6-Methyl-2-pyridinyl)-N-phenyl-4-(4-quinolinyl)-1H-pyrazole-1-carbothioamide (A 83-01), 4-[4-(2,3-Dihydro-1,4-benzodioxin-6-yl)-5-(2-pyridinyl)-1H-imidazol-2-yl]benzamide (D 4476), 4-[4-[3-(2-Pyridinyl)-1H-pyrazol-4-yl]-2-pyridinyl]-N-(tetrahydro-2H-pyran-4-yl)-benzamide (GW 788388), 4-[3-(2-Pyridinyl)-1H-pyrazol-4-yl]-quinoline (LY 364847), 4-[2-Fluoro-5-[3-(6-methyl-2-pyridinyl)-1H-pyrazol-4-yl]phenyl]-1H-pyrazole-1-ethanol (R 268712) or 2-(3-(6-Methylpyridine-2-yl)-1H-pyrazol-4-yl)-1,5-naphthyridine (RepSox).

d. FGF Inhibitors

Basic fibroblast growth factor (also known as bFGF, FGF2 or FGF-β) is a member of the fibroblast growth factor family. bFGF is present in basement membranes and in the subendothelial extracellular matrix of blood vessels. In addition, bFGF is a common component of human pluripotent cell culture medium in which it is necessary for the cells to remain in an undifferentiated state. An inhibitor of the FGF pathway can also include inhibitors of related signal transduction pathways including but not limited to, e.g., the MAPK/ERK signal transduction pathway.

A bFGF inhibitor refers to bFGF inhibitors in general. For example, bFGF inhibitors include, but are not limited to N-[2-[[4-(Diethylamino)butyl]amino-6-(3,5-dimethoxyphenyl)pyrido[2,3-d]pyrimidin-7-yl]-N′-(1,1-dimethylethyl)urea (PD173074), 2-(2-Amino-3-methoxyphenyl)-4H-1-benzopyran-4-one (PD 98059), 1-tert-Butyl-3-[6-(2,6-dichlorophenyl)-2-[[4-(diethylamino)butyl]amino]pyrido[2,3-d]pyrimidin-7-yl]urea (PD161570), 6-(2,6-Dichlorophenyl)-2-[[4-[2-(diethylamino)ethoxy]phenyl]amino]-8-methyl-pyrido[2,3-d]pyrimidin-7(8H)-one dihydrochloride hydrate (PD166285), N-[2-Amino-6-(3,5-dimethoxyphenyl)pyrido[2,3-d]pyrimidin-7-yl]-N′-(1,1-dimethylethyl)-urea (PD166866), and MK-2206.

In some aspects, inhibitors of the FGF pathway are also inhibitors of the MAPK/ERK pathway and include but are not limited to, e.g., AP 24534 (3-(2-Imidazo[1,2-b]pyridazin-3-ylethynyl)-4-methyl-N-[4-[(4-methyl-1-piperazinyl)methyl]-3-(trifluoromethyl)phenyl]-benzamide), PD173074 (N-[2-[[4-(Diethylamino)butyl]amino]-6-(3,5-dimethoxyphenyl)pyrido[2,3-d]pyrimidin-7-yl]-N′-(1,1-dimethylethyl)urea), FUN 1 hydrochloride (N-(3-((3-(2,6-dichloro-3,5-dimethoxyphenyl)-7-(4-(diethylamino)butylamino)-2-oxo-3,4-dihydropyrimido[4,5-d]pyrimidin-1(2H)-yl)methyl)phenyl)acrylamide), PD 161570 (N-[6-(2,6-Dichlorophenyl)-2-[[4-(diethylamino)butyl]amino]pyrido[2,3-d]pyrimidin-7-yl]-N′-(1,1-dimethylethyl)urea), SU 5402 (2-[(1,2-Dihydro-2-oxo-3H-indol-3-ylidene)methyl]-4-methyl-1H-pyrrole-3-propanoic acid), SU 6668 (5-[1,2-Dihydro-2-oxo-3H-indol-3-ylidene)methyl]-2,4-dimethyl-1H-pyrrole-3-propanoic acid), PD0325901 (N-[(2R)-2,3-Dihydroxypropoxy]-3,4-difluoro-2-[(2-fluoro-4-iodophenyl)amino]-benzamide), BIX 02189 ((3Z)-3-[[[3-[(Dimethylamino)methyl]phenyl]amino]phenylmethylene]-2,3-dihydro-N,N-dimethyl-2-oxo-1H-indole-6-carboxamide), FR 180204 (5-(2-Phenyl-pyrazolo[1,5-a]pyridin-3-yl)-1H-pyrazolo[3,4-c]pyridazin-3-ylamine), Pluripotin (N-[3-[7-[(1,3-Dimethyl-1H-pyrazol-5-yl)amino]-1,4-dihydro-1-methyl-2-oxopyrimido[4,5-d]pyrimidin-3(2H)-yl]-4-methylphenyl]-3-(trifluoromethyl)benzamide), TCS ERK 11e (4-[2-[(2-Chloro-4-fluorophenyl)amino]-5-methyl-4-pyrimidinyl]-N-[(1S)-1-(3-chlorophenyl)-2-hydroxyethyl]-1H-pyrrole-2-carboxamide), TMCB (2-(4,5,6,7-Tetrabromo-2-(dimethylamino)-1H-benzo[d]imidazol-1-yl)acetic acid), XMD 8-92 (2-[[2-Ethoxy-4-(4-hydroxy-1-piperidinyl)phenyl]amino]-5,11-dihydro-5,11-dimethyl-6H-pyrimido[4,5-b][1,4]benzodiazepin-6-one), SU5402, AZD4547, BGJ398, AL 8697, AMG 548, CMPD-1, DBM 1285 dihydrochloride, EO 1428, JX 401, ML 3403, RWJ 67657, SB 202190, SB-203580, SB 239063, SB 706504, Scio-469, SKF 86002 dihydrochloride, SX 011, TA 01 (4-(2-(2,6-Difluorophenyl)-4-(fluorophenyl)-1H-imidazol-5-yl)pyridine), TA 02 (4-(2-(2-Fluorophenyl)-4-(fluorophenyl)-1H-imidazol-5-yl)pyridine), TAK 715, VX-702, VX-745, antibodies to FGF and/or MAPK pathway components including ligands and receptors, FGF and/or MAPK inhibitory nucleic acids, and the like.

e. PI3K Inhibitors

Phosphoinositide 3-kinase (PI3K) inhibitors are a class of medicines that have been developed to inhibit one or more of the phosphoinositide 3-kinase enzymes. These enzymes form part of the PI3K/AKT/mTOR pathway, which is a pathway involved in cell growth and survival. There are pan-class I PI3K inhibitors such as copanlisib (2-Amino-N-[7-methoxy-8-(3-morpholin-4-ylpropoxy)-2,3-dihydroimidazo[1,2-c]quinazolin-5-yl]pyrimidine-5-carboxamide), isoform-specific PI3K inhibitors such as idelalisib (5-fluoro-3-phenyl-2-[(1S)-1-(7H-purin-6-ylamino)propyl]quinazolin-4-one), and dual PI3K/mTOR inhibitors such as dactolisib ((613C)cyclohexatrienecarboxylic acid).

Inhibitors of the PI3K pathway include but are not limited to, e.g., AS 252424 (5-[[5-(4-Fluoro-2-hydroxyphenyl)-2-furanyl]methylene]-2,4-thiazolidinedione), AS 605240 (5-(6-Quinoxalinylmethylene)-2,4-thiazolidine-2,4-dione), AZD 6482 ((−)-2-[[(1R)-1-[7-Methyl-2-(4-morpholinyl)-4-oxo-4H-pyrido[1,2-a]pyrimidin-9-yl]ethyl]amino]benzoic acid), BAG 956 (α,α,-Dimethyl-4-[2-methyl-8-[2-(3-pyridinyl)ethynyl]-1H-imidazo[4,5-c]quinolin-1-yl]-benzeneacetonitrile), CZC 24832 (5-(2-Amino-8-fluoro[1,2,4]triazolo[1,5-a]pyridin-6-yl)-N-(1,1-dimethylethyl)-3-pyridinesulfonamide), GSK 1059615 (5-[[4-(4-Pyridinyl)-6-quinolinyl]methylene]-2,4-thiazolidenedione), KU 0060648 (4-Ethyl-N-[4-[2-(4-morpholinyl)-4-oxo-4H-1-benzopyran-8-yl]-1-dibenzothienyl]-1-piperazineacetamide), LY 294002 hydrochloride (2-(4-Morpholinyl)-8-phenyl-4H-1-benzopyran-4-one hydrochloride), 3-Methyladenine (3-Methyl-3H-purin-6-amine), PF 04691502 (2-Amino-8-[trans-4-(2-hydroxyethoxy)cyclohexyl]-6-(6-methoxy-3-pyridinyl)-4-methyl-pyrido[2,3-d]pyrimidin-7(8H)-one), PF 05212384 (N-[4-[[4-(Dimethylamino)-1-piperidinyl]carbonyl]phenyl]-N′-[4-(4,6-di-4-morpholinyl-1,3,5-triazin-2-yl)phenyl]urea), PI 103 hydrochloride (3-[4-(4-Morpholinylpyrido[3′,2′:4,5]furo[3,2-d]pyrimidin-2-yl]phenol hydrochloride), PI 828 (2-(4-Morpholinyl)-8-(4-aminophenyl)-4H-1-benzopyran-4-one), PP 121 (1-Cyclopentyl-3-(1H-pyrrolo[2,3-b]pyridin-5-yl)-1H-pyrazolo[3,4-d]pyrimidin-4-amine), Quercetin, TG 100713 (3-(2,4-Diamino-6-pteridinyl)-phenol), Wortmannin, PIK90, GDC-0941, antibodies to PI3K and PI3K receptors, PI3K inhibitory nucleic acids, and the like.

f. Wnt Activators

Signaling by the Wnt family of secreted glycoproteins plays important roles in embryonic development and adult homeostasis. Wnt signaling is modulated by a number of evolutionarily conserved inhibitors and activators. Endogenous activators include R-spondin and Norrin, and recently, secreted frizzled-related protein-2 (sFRP2) (de Castro et al, Bone Research, 2021). Synthetic activators include Wnt agonist (N4-(1,3-Benzodioxol-5-ylmethyl)-6-(3-methoxyphenyl)-2,4-pyrimidinediamine), WAY 262611 (1-(4-(Naphthalen-2-yl)pyrimidin-2-yl)piperidin-4-yl)methanamine) and LP 922056 (2-[(6-Chloro-7-cyclopropylthieno[3,2-d]pyrimidin-4-yl)thio]acetic acid).

Activators of the WNT pathway include but are not limited to, CHIR99021 (6-[[2-[[4-(2,4-Dichlorophenyl)-5-(5-methyl-1H-imidazol-2-yl)-2-pyrimidinyl]amino]ethyl]amino]-3-pyridinecarbonitrile), Wnt family ligands (e.g., including but not limited to Wnt-1, Wnt-2, Wnt-2b, Wnt-3a, Wnt-4, Wnt-5a, Wnt-5b, Wnt-6, Wnt-7a, Wnt-7a/b, Wnt-7b, Wnt-8a, Wnt-8b, Wnt-9a, Wnt-9b, Wnt-10a, Wnt-10b, Wnt-11, Wnt-16b, etc.), RSPO co-agonists (e.g., RSPO2), lithium chloride, TDZD8 (4-Benzyl-2-methyl-1,2,4-thiadiazolidine-3,5-dione), BIO-Acetoxime ((2′Z,3′E)-6-Bromoindirubin-3′-acetoxime), A1070722 (1-(7-Methoxyquinolin-4-yl)-3-[6-(trifluoromethyl)pyridin-2-yl]urea), HLY78 (4-Ethyl-5,6-Dihydro-5-methyl-[1,3]dioxolo[4,5-j]phenanthridine), CID 11210285 hydrochloride (2-Amino-4-(3,4-(methylenedioxy)benzylamino)-6-(3-methoxyphenyl)pyrimidine hydrochloride), WAY-316606, (hetero)arylpyrimidines, IQ1, QS11, SB-216763, DCA, and the like. In some instances, activation of the Wnt pathway may be achieved through repression of a Wnt pathway inhibitor, e.g., including but not limited to the use of an inhibitory nucleic acid targeting an inhibitor of the Wnt pathway or an antibody or small molecule directed to a Wnt pathway inhibitor.

g. Hedgehog Activators

Activators of the Hedgehog pathway include but are not limited to, e.g., Hedgehog family ligands (Hh, Shh, Ihh, Dhh, etc.) and fragments thereof, benzothiophene smoothened agonists, SAG (Hh-Ag1.3), SAG21k (3-chloro-4,7-difluoro-N-(4-methoxy-3-(pyridin-4-yl)benzyl)-N-((1r,4r)-4-(methylamino)cyclohexyl)benzo[b]thiophene-2-carboxamide), Hh-Ag1.1, Hh-Ag1.5, purmorphamine, and the like. In some instances, activation of the Hedgehog pathway may be achieved through repression of the a Hedgehog pathway inhibitor, e.g., including but not limited to the use of an inhibitory nucleic acid targeting an inhibitor of the Hedgehog pathway or an antibody or small molecule directed to a Hedgehog pathway inhibitor.

In some instances, Hedgehog pathway activators include those agents described in, e.g., Chen et al. (2002) PNAS, 99(22):14071-14076; Frank-Kamenetsky, et al. (2002) J Biol. 1(2):10; Paladini et al. (2005) J Invest Dermatol. 125(4):638-46; Nakamura et al. (2014) J Cell. Physiol. ePub, Yun et al., Arch Pharm Res. 2012 August; 35(8):1317-33.

In further aspects, the Hedgehog activator is 20(S)-Hydroxycholesterol ((3β)-Cholest-5-ene-3,20-diol), SAG (3-Chloro-N-[trans-4-(methylamino)cyclohexyl]-N-[[3-(4-pyridinyl)phenyl]methyl]benzo[b]thiophene-2-carboxamide), or SAG-21K (3-Chloro-4,7-difluoro-N-[[2-methoxy-5-(4-pyridinyl)phenyl]methyl]-N-trans-4 (methylamino)cyclohexyl]benzo[b]thiophene-2-carboxamide).

h. BMPR1A/B Agonists

BMPR1A agonists include GDF5, BMP-4, BMP-5, BMP-6, BMP-7, BMP-8A, BMP-8B, BMP-9, BMP-10, BMP-11, GDF-6, GDF-7, and anti-Müllerian hormone (AMH). These are of use in the disclosed methods. Additionally, synthetic molecules that activate BMP signaling can be used, such as Kielin/chordin-like proteins (KCPs).

### Methods of Treatment

Chondrocytes derived from an iPSC, as disclosed herein, including conjugates of the chondrocytes attached to a solid carrier, can be administered locally at a site in a subject wherein the formation of stable cartilage is desirable. Suitable solid carriers are disclosed above. In some aspects, the solid carrier is HyA-FMBs. In some aspects, the solid carrier is hyaluronic acid. In further aspects, the solid carrier is biodegradable. However, the disclosed conjugates derived from a hiPSC can be used without a solid carrier, such as in the form of a pharmaceutical composition, or with a hydrogel, extracellular matrix, or other porous substrate.

In some aspects, a method is provided for promoting cartilage growth and/or repair, that includes administering locally to a site in a subject in need thereof, a therapeutically effective amount of chondrocytes derived from an hiPSC, thereby producing stable cartilage locally at the site in the subject. In more aspects, the subject has, or is at risk of having, cartilage damage or degradation at the site.

The site may be intra-articular; e.g., at a joint, for example hip, knee, shoulder, ankle, wrist, elbow, etc. Routes of administration include injection or minimally invasive surgery. In aspects, an injection is an ultrasound guided injection. Minimally invasive surgery, also referred to as arthroscopic surgery, bandaid surgery, or keyhole surgery is a surgical procedure meant to minimize tissue damage and ease recovery. Minimally invasive surgery can be robotic, or non-robotic. However, the surgery may not be minimally invasive. The surgery may be conventional. The surgery can be robotic assisted, such as a robotic assisted joint replacement. Non-natural human chondrocytes attached to HyA-FMBs can be used at the time of a major surgery, such as during a joint surgery or replacement, or when a bone is set.

By way of example, one method of administration to the knee, hip and/or shoulder of an individual is by intra-articular injection. For administration to the knee, for example, the joint to be injected is washed with a betadine solution or other antiseptic. A solution of an anesthetic, such as about one percent lidocaine hydrochloride is injected into the skin and subcutaneous tissue. A 3-way stopcock/needle assembly is utilized to administer the compound via an 18-30 gauge needle. The chondrocytes attached to the solid carrier, such as fibrin (for example, HyA-FMB) are injected into the joint space using a standard lateral approach. The needle and needle tract are cleansed by flushing with 1% lidocaine hydrochloride through the 3-way stopcock assembly as the needle is withdrawn. The knee is then moved through a flexion-extension are and then immobilized in full extension. The patient is then confined to bed for approximately 24 hours to minimize movement and minimize leakage of HyA-FMBs from the joint.

In aspects, an individual administration can include about 5 mg, 10 mg, 15 mg, 20 mg, 30 mg, 40 mg, 50 mg, 60 mg, 70 mg, 80 mg, 90 mg, 100 mg, or more milligrams of chondrocytes attached to a solid carrier, such as fibrin, such as HyA-FMBs. An individual administration can include at least 1×10⁴, 1×10⁵, 1×10⁶, 1×10⁷, 1×10⁸ or more cells. Administration may be a single administration or multiple administrations, such as 2, 3, 4, 5, or more administrations. Multiple administrations may be separated by at least 1 day, 2 days, 3 days, 4 days, 5 days, 6 days, 1 week, 2 weeks, I month, 2 months, 3 months, 6 months, 1 year, or more. In aspects, administration can be to one or more joints.

The disclosed compositions be administered in conjunction with, following, or prior to, treatment with anti-inflammatory agents; e.g., non-steroidal anti-inflammatories. Non-steroidal anti-inflammatories include, but are not limited to, salicylates, propionic acid derivatives, acetic acid derivatives, enolic acid derivatives, anthranilic acid derivatives, selective COX-2 inhibitors, sulfonanilides. Common non-steroidal anti-inflammatories include, for example, aspirin, ibuprofen and naproxen.

In particular examples, anti-arthritis agents can be used. In some aspects, the anti-arthritis agent is a biological response modifier, such as KINERET® (anakinra), ENBREL® (etanercept), or REMICADE® (infliximab), a disease-modifying antirheumatic drug (DMARD), such as ARAVA® (leflunomide), a steroid, such as prednisone or cortisone, a nonsteroidal anti-inflammatory drug (NSAID), such as celecoxib, choline magnesium trisalicylate, diclofenac, diclofenac potassium, diclofenac XR, diflunisal, etodolac, etodolac ER, fenoprofen, flurbiprofen oral, ibuprofen, indomethacin, indomethacin SR, indomethacin suppositories, ketoprofen, ketoprofen ER, meclofenamate, meloxicam, nabumetone, naproxen, naproxen CR, naproxen ER, oxaprozin, piroxicam, rofecoxib, salsalate, sulindac, or tolmetin sodium, or another product, such as HYALGAN® (hyaluronan) or SYNVISC® (hylan G-F20).

### A. Stable Cartilage

Methods of the present disclosure are useful in promoting cartilage growth and/or repair by producing stable cartilage. In aspects, administration of chondrocytes attached to a solid carrier, such as fibrin (for example, HyA-FMBs) can produce cartilage at and/or around the administration site. In aspects, the cartilage is articular cartilage. In aspects, the presence of cartilage can be determined by the expression of Collagen Type 11, and/or aggrecan. In aspect, the expression of collagen Type X indicates non-stable cartilaginous tissues. Cartilage is radiolucent, however, clinically physicians can measure the distance between the boney epiphyses (joint space) as a surrogate to measure cartilage. In some aspect, the non-natural human chondrocyte does not undergo hypertrophy for at least 5 months when transplanted in vivo. In some aspects, the non-natural chondrocytes do not undergo hypertrophy for a at least about 5, 6, 7, 8, 9, 10, 11 or 12 months in vivo. In more aspects, the non-natural chondrocytes do not undergo hypertrophy for a at least 5, 6, 7, 8, 9, 10, 11 or 12 months in vivo.

Cartilage is sometimes the initial stage in the development of ossified tissue in vivo following cell transplantation (it goes on to hypertrophy before forming bone). Prior studies using pre-treated BMSCs (e.g. treated with cartilage inducing factors in vitro) that are then transplanted in vivo have failed to form stable cartilage in vivo. The methods of the present disclosure utilize chondrocytes derived from iPSC, as disclosed herein. Cartilage produced by methods of the present disclosure is not a cartilage that goes on to hypertrophy before the development of bone, or other ossified tissue, but persists in a cartilage state. In some aspects, this stable cartilage persists in vivo for about 5 months, about 6 months, about 7 months, about 8 months, about 9 months, about 10 months, about 11 months, about one year, or more following administration. In aspects, stable cartilage can resist vascularization in vivo.

### B. Subjects and Administration

A subject in need of the present methods can be those at risk of developing, or having, osteoarthritis, osteochondritis dissecans, osteochondrodysplasias, or cartilage injury. A subject in need thereof can additionally have bone damage; e.g., from osteoarthritis or accidental injury. The subject can have, or be at risk of developing, osteoarthritis, osteochondritis dissecans, osteochondrodysplasia, or cartilage injury. The subject can have a cartilage defect.

Subjects include both human and veterinary subjects, such as humans, non-human primates, cats, dogs, pigs, sheep, cows, horses, rodents, birds, and the like, which can be the recipient of the disclosed methods. In some examples, the subject is human. The subject can be an adult subject. The subject can be a child or an infant.

Osteoarthritis is a type of joint discase resultant from the breakdown of cartilage and bone within the joints. Osteoarthritis causes joint pain and stiffness, swelling and decreased range of motion.

Cartilage Injury includes any injury or damage to the cartilage tissue. The cartilage can be articular cartilage. Cartilage injuries include tears, rips and ruptures. Cartilage injury typically can occur in the joints; e.g., knee, hip, elbow, shoulder, ankle, etc. Exemplary cartilage injuries include meniscal tears, labral tears of the hip or shoulder, talar dome lesions, and others. Cartilage injuries can occur due to accident, athletic injury, disease, genetic conditions, and other conditions. In an aspect, MHC-matched chondrocytes derived from iPSC, linked to a solid carrier, such a fibrin (for example, but not limited to, HyA-FMB) are injected into cartilage lesions, such as at the site of a cartilage injury.

Methods of the present disclosure can be utilized in the growth or repair of cartilage for subjects in need thereof. Growth or repair may include partial or complete regrowth of cartilage. Partial cartilage regrowth may include about at least 10%, 20%, 30%, 40%, 50%, 60%, 70%, 80%, 90%, 95%, 99%, or 100% regrowth of cartilage. Cartilage regrowth may be measured as a percentage of weight, or percentage of a distance, for example a cartilage length, width, diameter, or height. Repair can be measured as a percentage extension in length, height, width, diameter, or weight. Repair can further be measured as an increase in joint mobility, or subjectively, as in increase in joint comfort.

In one aspect, administration is local, such as intra-articular. The administration can be via an injection or a minimally invasive or conventional surgical procedure. Administration can be to a joint, such as, but not limited to, a knee, shoulder, wrist or hip. The administration can be using a minimally invasive surgical procedure or a standard surgical procedure.

In another aspect, administration is local such as by intramedullary injection. Intramedullary administration can be achieved by direct injection into the marrow space of a fracture site, without injection into the periosteum or bone cortex. Intramedullary administration can be administered by direct injection into the marrow, or by insertion into a hole made by a K-wire through the intramedullary canal. Administration also can be trans-osseous or locally to the periosteum. Administration can be via a standard surgical procedure or a minimally invasive procedure.

The composition can be administered once but may be applied periodically until either a therapeutic result is achieved or until side effects warrant discontinuation of therapy. In one example, a dose is infused over time. In a one example, a continuous infusion is administered for about one to about ten days, such as for about two to five days, such as 1, 2, 3, 4, 5, 6, 7, 8, 9, or 10 days. In another example, a dose of an agent is administered as a bolus one or more times. In some embodiments, a single administration is provided to the subject.

The subject can be treated at regular intervals, such as daily, biweekly, weekly, bi-monthly, or monthly, until a desired therapeutic result is achieved. Generally, the dose is sufficient to treat or ameliorate symptoms or signs of disease without producing unacceptable toxicity to the patient. Amounts effective for this use will depend upon the activity of the agent, the severity of the disease and the general state of the patient's health. A therapeutically effective amount provides either subjective relief of a symptom(s) or an objectively identifiable improvement as noted by the clinician or other qualified observer. Combinations of agents are also envisioned. Administration may begin whenever the suppression or prevention of disease is desired.

For treatment of a subject, depending on activity of the agent, manner of administration, nature and severity of the disorder, age and body weight of the patient, different doses are necessary. Under certain circumstances, however, higher or lower doses may be appropriate. The administration of the dose can be carried out both by single administration in the form of an individual dose unit or else several smaller dose units and also by multiple administrations of subdivided doses at specific intervals. A skilled clinician can readily determine an effective dose.

In some aspects, for local administration to bone, a scaffold is utilized, which includes, for example, hydroxyapatite or a combination of polylactic acid and glycolic acid. By varying the proportion of the two components is polymers of different mechanical properties are obtained. Thus, in several aspects, the ratio of polylactic acid:glycolic acid is about 1:1, about 2:1, about 3:1 or about 4:1. In one example, the scaffolding includes about 75% polylactic acid and about 25% glycolic acid.

In another aspect, the scaffold is porous. For example, a scaffold can be about 85%, about 90%, about 95%, about 98% porous, such as for non-weight bearing tissue. In additional examples, the scaffold is about 5% porous, about 10% porous, about 15% porous or about 20% porous, such as for weight bearing tissue. The porosity can be determined, for example, by the fusing of micro spheres with CO₂ treatment. In this process commercial pellets of the polymer are converted to microspheres of the desired size, which are fused to develop a porous structure. By altering the micropore size scaffolds of different microporosity can be obtained.

In some aspects subject can have bone damage. Methods are provided to promote fracture healing. The fracture can be in any bone, including but not limited to cranial bones such as the frontal bone, parietal bone, temporal bone, occipital bone, sphenoid bone, ethmoid bone; facial bones such as the zygomatic bone, superior and inferior maxilla, nasal bone, mandible, palantine bone, lacrimal bone, vomer bone, the inferior nasal conchae; the bones of the ear, such as the malleus, incus, stapes; the hyoid bone; the bones of the shoulder, such as the clavicle or scapula; the bones of the thorax, such as the sternum or the ribs; the bones of the spinal column including the cervical vertebrae, lumbar vertebrae, and thoracic vertebrae; the bones of the arm, including the humerus, ulna and radius; the bones of the hands, including the scaphoid, lunate, triquetrum bone, pisiform bone, trapezium bone, trapezoid bone, capitate bone, and hamate bone; the bones of the palm such as the metacarpal bones; the bones of the fingers such as the proximal, intermediate and distal phalanges the bones of the pelvis such as the ilium, sacrum and coccyx; the bones of the legs, such as the femur, tibia, patella, and fibula; the bones of the feet, such as the calcaneus, talus, navicular bone, medial cuneiform bone, intermediate cuneiform bone, lateral cuneiform bone, cuboidal bone, metatarsal bone, proximal phalanges, intermediate phalanges and the distal phalanges; and the pelvic bones. In some aspects, the method includes administering a therapeutically effective amount of the chondrocytes derived from a hiPSC to a subject with these disorders, thereby treating the disorder.

Methods are also provided to promote spinal fusion. Spinal fusion can be induced in any of the vertebrae, including, but not limited to, the cervical vertebrae, lumbar vertebrae, and thoracic vertebrae. In one example, spinal fusion occurs in the absence of extra-skeletal bone formation, such as in the absence of bone formation in the soft tissues. In some aspects, the method includes administering a therapeutically effective amount of the chondrocytes derived from a hiPSC to a subject with these disorders.

In additional aspects, the methods disclosed herein can be used to treat subjects that have a broken bone due to any disease, defect, or disorder which affects bone strength, function, and/or integrity, such as decreasing bone tensile strength and modulus. Examples of bone diseases include, but are not limited to, diseases of bone fragility, such as osteoporosis and osteoarthritis. In some aspects, the method includes administering a therapeutically effective amount of the chondrocytes derived from a hiPSC to a subject with these disorders.

In some aspects, a therapeutically effective dose is the quantity necessary to induce bone growth, to support bone growth, or to heal a fracture. Exemplary assays to determine if a method treats the bone defect include radiographic methods (Lehmann et al., Bone 35: 1247-1255, 2004: Rundle et al., Bone 32: 591-601, 2003; Nakamura et al., J. Bone Miner. Res. 13: 942-949, 1998); microcomputed tomography (μCT) methods (Nakamura et al., J. Bone Miner. Res. 13: 942-949, 1998; Lehmann et al., Bone 35: 1247-1255, 2004; Tamasi et al., J. Bone Miner. Res. 18: 1605-1611, 2003; Shefelbine et al., Bone 36:480-488, 2005); peripheral quantitative computed tomographic methods (Rundle et al., Bone 32: 591-601, 2003; Tamasi et al., J. Bone Miner. Res. 18: 1605-1611, 2003); dual energy X-ray absorptiometry methods (Holzer et al., Clin. Orthop. Rel. Res. 366: 258-263, 1999; Nakamura et al., J. Bone Miner. Res. 13: 42-949, 1998); histomorphometry methods (Lehmann et al., Bone 35: 247-1255, 2004; Tamasi et al., J. Bone Miner. Res. 18:1605-1611, 2003; Li et al., J. Bone Miner. Res. 17: 791-799, 2002; Schmidmaier et al., Bone 30: 816-822; 2002; Nakamura et al., J. Bone Miner. Res. 13:942-949, 1998; Sheng et al., Bone 30: 486-491, 2002); Masson's trichrome stain for collagen (Rundle et al., Bone 32: 591-601, 2003); Goldner's stain for collagen (Holzer et al., Clin. Orthop. Rel. Res. 366: 258-263; 1999); Von Kossa's silver stain for bone (Schmidmaier et al., Bone 30: 816-822, 2002); Safranin Orange stain for collagen (Schmidmaier et al., Bone 30: 816-822, 2002); and immunohistochemistry methods (Rundle et al., Bone 32: 591-601, 2003; Li et al., J. Bone Miner. Res. 17: 791-799, 2002; Safadi et al., J. Cell Physiol. 196: 51-62, 2003; Iwaki et al., J. Bone Miner. Res. 12: 96-102, 1997). In some aspects, the method includes administering a therapeutically effective amount of the chondrocytes derived from a hiPSC to a subject with these disorders.

Other agents can also be administered, such as chemical compounds. In one aspect, an anti-inflammatory agent, such as a non-steroidal anti-inflammatory agent, is administered to the subject. In another aspect, an antibiotic, antifungal, or anti-viral agent is administered to the subject. Thus, other therapeutic agents can also be utilized in the disclosed methods, to promote fracture healing and/or spinal fusion.

When the subject has osteoporosis, the method can further include administering a therapeutically effective amount of a bisphosphonate or calcitonin. In further aspects, the method includes administering a therapeutically effective amount of a bisphosphonate, an antibody that specifically binds receptor activator of nuclear factor kappa-B ligand (RANKL), and/or a teriparatide. In a specific non-limiting example, the antibody that specifically binds RANKL is denosumab. Examples of bisphosphonates include, but are not limited to, zoledronic acid, pamidronate, ibandronate, alendronate, and risedronate.

The following examples are provided to illustrate particular features of certain aspects of the disclosure, but the scope of the claims should not be limited to those features exemplified.

### EXAMPLES

Development of articular cartilage during embryonic and postnatal life can provide insight on pathways that can be employed in engineering efficacious cell therapies. Following conception and the formation of three germ layers during gastrulation, embryonic mesenchyme condenses and secretes a template of Type I collagen in an area that will become a long bone (De Kinderen et al., Journal of Bone and Mineral Research, 37(3), 397-410, doi.org/10.1002/jbmr.4524, 2022). The induction of Sex-Determining Region Y-Box (SOX)-family transcription factors—SOX5, SOX6, and SOX9—promotes chondrogenic differentiation, leading to the formation of a cartilage template rich in Type II collagen and Aggrecan. The majority of chondroprogenitors undergo hypertrophy, begin secreting Type X collagen, and either go through apoptosis or become osteogenic, resulting in the replacement by bone in a process called endochondral ossification (Yang et al., Proceedings of the National Academy of Sciences of the United States of America, 111(33), 12097-12102. doi.org/10.1073/pnas.1302703111, 2014). However, a condensation of flattened cells in the presumptive joint area—known as the interzone—begins to regulate the development of articular chondrocytes that resist hypertrophy (Archer et al., Birth Defects Research Part C—Embryo Today: Reviews, 69(2), 144-155. doi.org/10.1002/bdrc.1001, 2003). The interzone is rich in proliferation-deficient cells that secrete TGFβ, GDF-5, and Wnt ligands, while a nearby area termed the distal proliferative zone contains cells that are exposed to BMP inhibitors, such as Noggin, and progressively migrate to the interzone region (Pacifici et al., Annals of the New York Academy of Sciences, 1068(1), 74-86, doi.org/10.1196/annals.1346.010, 2006; Ray et al., Development (Cambridge), 142(6), 1169-1179, doi.org/10.1242/dev.110940, 2015; Spagnoli et al., Journal of Cell Biology, 177(6), 1105-1117, doi.org/10.1083/jcb.200611031, 2007). During joint cavitation, interzone cells synthesize large amounts of hyaluronic acid, which binds to CD44 and Aggrecan, the latter of which forms aggregates with anionic side chains that attract water into the developing matrix (Archer et al., supra, 2003; De Kinderen et al., supra, 2022). A layered architecture begins to appear in articular cartilage. Lubricin (PRG4) becomes preferentially expressed at the surface layer of articular cartilage to reduce friction with movement, and interacts with Cartilage Oligomeric Matrix Protein (COMP) and Type II collagen expressed in both surface and deeper layers (Flowers et al., Scientific Reports, 7(1), 1-11, doi.org/10.1038/s41598-017-13558-y, 2017; Maly et al., International Journal of Molecular Sciences, 22(5), 1-23, doi.org/10.3390/ijms22052242, 2021). The surface layer is also marked by the expression of Bone Morphogenetic Protein Receptor Type-1B (BMPR1B), the receptor for GDF-5. The surface (superficial) layer is typically the most damaged by OA, displaying fibrillations and fissures with joint ageing and OA, leading to the exposure of deeper layers and subchondral bone that elicit pain with joint movement (Wu et al., The Innovation, 2(3), 100141, doi.org/10.1016/j.xinn.2021.100141, 2013). Because BMP signaling plays key roles in interzone development and surface articular cartilage homeostasis, it is wondered whether this pathway can be fine-tuned in stem cell differentiation strategies.

One stem cell population that has been vigorously tested for OA regenerative therapies are hBMSC/SSC, a subset of pericytes that are derived from perichondral cells during endochondral ossification that attach to invading blood vessels, where they remain in their primitive state in adult marrow (Robey & Riminucci, Skeletal stem cells: tissue-specific stem/progenitor cells of cartilage, bone, stroma, and marrow adipocytes. In: Principles of Bone Biology (4th ed., pp. 45-71). Academic Press 2020). Adult BMSCs/SSCs are identified by their ability to generate bone, hematopoietic-supporting stroma, and adipocytes in transplantation studies, and the ability to self-renew across serial transplantation (Bianco et al., Nat. Med. 19, 35-42. 10.1038/nm.3028, 2013) Marrow hBMSCs/SSCs do not directly form cartilage during development or in the steady state, but maintain a chondrogenic memory, as their chondrogenic capacity is revealed upon in vitro pellet culture conditions supplemented with TGFβ. However, current chondrogenic induction strategies of hBMSCs/SSCs seem to follow a default path of endochondral ossification, evidenced by the expression of hypertrophic markers in vitro and matrix calcification in vivo, questioning whether hBMSCs/SSCs have the potential to form surface hyaline cartilage (Pelttari et al., Arthritis and Rheumatism, 54(10), 3254-3266, doi.org/10.1002/art.22136, 2006; Scotti et al., Proceedings of the National Academy of Sciences of the United States of America, 110(10), 3997-4002, doi.org/10.1073/pnas.1220108110, 2013; Somoza et al., Tissue Engineering—Part B: Reviews, 20(6), 596-608, doi.org/10.1089/ten.teb.2013.0771, 2014). One recent study has shown that the HyA-FMB scaffold promotes the differentiation of hBMSCs/SSCs to hyaline-like cartilage that is maintained for up to 28 weeks in vivo (Kuznetsov et al., Stem Cells Translational Medicine, 26, 586-592, doi.org/10.1016/j.joca.2018.02.29, 2019). Therefore, the HyA-FMB scaffold serves as an attractive model system to examine mechanisms that promote stable cartilage development and maintenance.

Another stem cell population that can be harnessed for OA regenerative therapies is the hiPSC. Since these cells can be reprogrammed from a patient's somatic cells and resist replicative senescence, hiPSCs offer advantages in preventing graft rejection, generating expandable and scalable tissues, and lack ethical restraints accompanied with similar strategies using embryonic stem cells (ESCs). Several differentiation strategies have been used to generate articular chondrocyte-like cells, including co-culture with chondrocytes (Bigdeli et al., Stem Cells, 27(8), 1812-1821. https://doi.org/10.1002/stem.114, 2009; Hwang et al., PLoS ONE, 3(6), doi.org/10.1371/joumal.pone.0002498, 2008), formation of a BMSC/SSC-like intermediate prior to chondrogenic differentiation (Chang et al., Stem Cells International, 2020, doi.org/10.1155/2020/8867349, 2020; Diederichs et al., Frontiers in Cell and Developmental Biology, 7 (November), 1-10, doi.org/10.3389/fcell.2019.002, 2019), and the stepwise differentiation that mimics embryonic gastrulation and formation of neural crest or mesoderm intermediates prior to chondrogenic induction (Chijimatsu et al., Stem Cells International, 2017, doi.org/10.1155/2017/1960965, 2017; Loh et al., Cell, 166(2), 451-467, doi.org/10.1016/j.cell.2016.06.011, 2016; Oldershaw et al., Nature Biotechnology, 28(11), 1187-1194, doi.org/10.1038/nbt.1683, 2010). Few chondrogenic differentiation strategies have yielded chondrocyte-like cells that are able to resist hypertrophy in functional transplantation studies (Lee et al., FASEB Journal, 29(8), 3399-3410, doi.org/10.1096/fj.14-269720m 2015; Wu et al., supra, 2021; Yamashita et al., Stem Cell Reports, 4(3), 404-418, doi.org/10.1016/j.stemcr.2015.01.016, 2015); however, these studies employed undefined, animal-derived factors (e.g., serum) in differentiation, hampering clinical translation. On the other hand, some serum-free strategies have shown effective chondrogenic differentiation in vitro (Craft et al., Nature Biotechnology, 33(6), 638-645, doi.org/10.1038/nbi.3210, 2015; Ferguson et al., Nature Communications, 9(1), doi.org/10.1038/s41467-018-05573-y, 2018), yet follow-up transplantation studies have focused on ESC-derived chondrocyte-like cells (Gardner et al., European Cells and Materials, 38(215), 215-227, doi.org/10.22203/eCM.v038a15, 2019; Petrigliano et al., Npj Regenerative Medicine, 6(1), doi.org/10.1038/s41536-021-00187-3, 2021). Therefore, the derivation of articular chondrocyte-like cells in serum-free and defined conditions that resist hypertrophy over long-term transplantation studies has been an ongoing challenge in hiPSC translational research.

Many studies have targeted the BMP signaling pathway in chondrogenic differentiation of hBMSCs/SSCs and hiPSCs; however, there is uncertainty whether BMP signaling is activated or repressed in the development of stable chondrocytes. Studies have shown that BMP-2 is induced after one day of TGFβ exposure in chondrogenic differentiation of hBMSCs/SSCs, suggesting that BMP-2 signaling may be skewing differentiation to hypertrophy and osteogenesis (Futrega et al., Communications Biology, 4(1), 1-12, doi.org/10.1038/s42003-020-01520-0, 2021). While blocking BMP signaling hampers mineralization in osteogenic cultures and chondrogenic cultures treated with β-Glycerophosphate (Hellingman et al., Tissue Engineering—Part A, 17(7-8), 1157-1167, doi.org/10.1089/ten.tea.2010.0043, 2011), chronic BMP blockade using LDN 193189 does not prevent hypertrophic expression in hBMSC/SSC chondrogenic differentiation (Franco et al., Stem Cell Reports, 17(3), 616-632. //doi.org/10.1016/j.stemcr.2022.01.0162022). However, transient BMP inhibition targeting ALK2 and ALK3 receptors during initial chondrogenic induction reduces hypertrophy and endochondral ossification associated with ectopic transplantation in vivo (Occhetta et al., Proceedings of the National Academy of Sciences of the United States of America, 115(18), 4625-4630, doi.org/10.1073/pnas.1720658115, 2018; Pelttari et al., Injury, 39(1 SUPPL.), 58-65, doi.org/10.1016/j.injury.2008.01.038, 2006), suggesting a role of BMP activation later in chondrogenic differentiation. Supporting this notion, BMP-2 has been shown to maintain native and transplanted articular cartilage, especially when vascularization is suppressed (Murphy et al., Nature Medicine, 26(10), 1583-1592. doi.org/10.1038/s41591-020-1013-2, 2020; Rountree et al., PLoS Biology, 2(11). doi.org/10.1371/journal.pbio.0020355, 2004). BMP activation is routine in the chondrogenic differentiation of hiPSCs, as several strategies demonstrate enhanced chondrogenesis when BMP-2, BMP-4, and/or GDF-5 are supplemented with TGFβ in chondrogenic induction medium (De Kinderen et al., supra, 2022; Yamashita et al., Stem Cell Reports, 4(3), 404-418, doi.org/10.1016/j.stemcr.2015.01.016, 2015). Recent studies have shown that BMP-4 promotes hypertrophic differentiation, leading some to conclude that BMP activation should be avoided in chondrogenic differentiation (Craft et al., Nature Biotechnology, 33(6), 638-645, doi.org/10.1038/nbt.3210, 2015; Diederichs et al., Frontiers in Cell and Developmental Biology, 7 (November), 1-10, doi.org/10.3389/fcell.2019.00270, 2019). However, early BMP inhibition followed by late BMP activation using GDF-5, similar to the joint interzone microenvironment, has not been tested in chondrogenic differentiation of hiPSCs.

It is demonstrated herein that HyA-FMBs promote early expression of non-collagenous proteins (i.e., COMP, DPT, IGFBP5, MGP) and BMP inhibitors (i.e., GREM1 and NBL1) with reduced expression of ID genes downstream of BMP activation in hBMSCs/SSCs. By days 5 and 10 of chondrogenic differentiation, however, a pre-chondrogenic subpopulation with high expression levels of IGFBP5 and MGP and low expression of hypertrophic and osteogenic markers shows an expression signature indicating restored and activated BMP signaling, which is accentuated in transplanted BMSC/SSC-derived stable chondrocytes. Applying this mechanistic knowledge to chondrogenic differentiation of hiPSCs, BMP signaling was inhibited during an initial sclerotome induction phase and activated BMP signaling using BMP-2 and GDF-5 during a subsequent chondrogenic induction phase. This biphasic BMP signaling produced chondrocyte-like cells with a stable phenotype in vitro when applied to a purified SOX9-expressing subpopulation of sclerotome cells, but produced a hypertrophic phenotype when applied to all sclerotome cells, indicating a time- and cell-specific nature of BMP signaling in stable chondrogenic differentiation. Further, treatment of these SOX9-expressing sclerotome cells with GDF-5 and BMP-2 in serum-free chondrogenic medium formed “chondrospheroids”, and when transplanted with HyA-FMBs into a femoral chondral defect led to stable hyaline-like cartilage for up to 5 months in NSG mice and SRG rats, offering an articular-like cell source for regenerative OA therapy.

### Example 1

### HyA-FMBs Promote Early Expression of Extracellular Matrix Genes

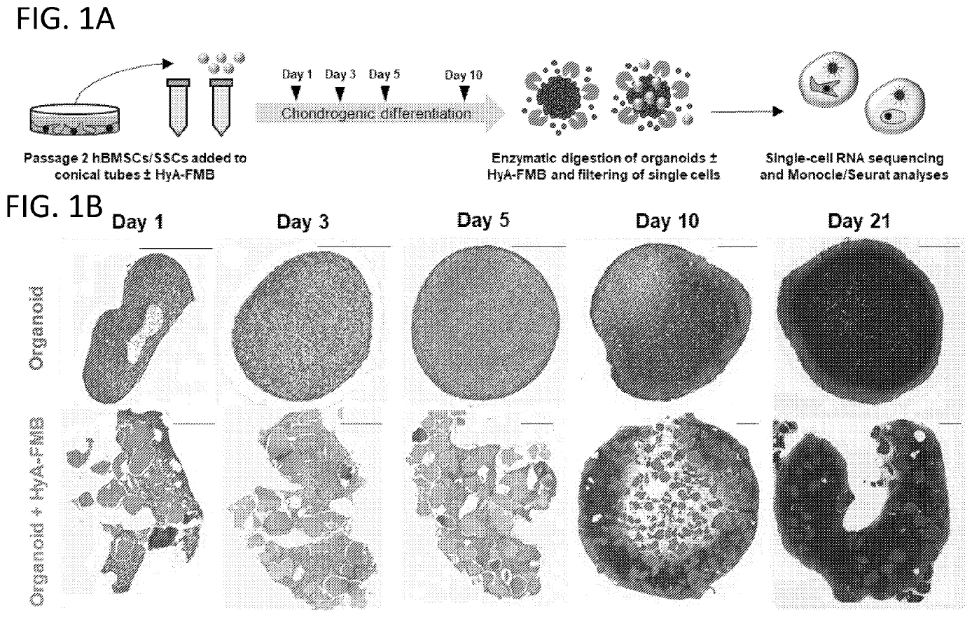

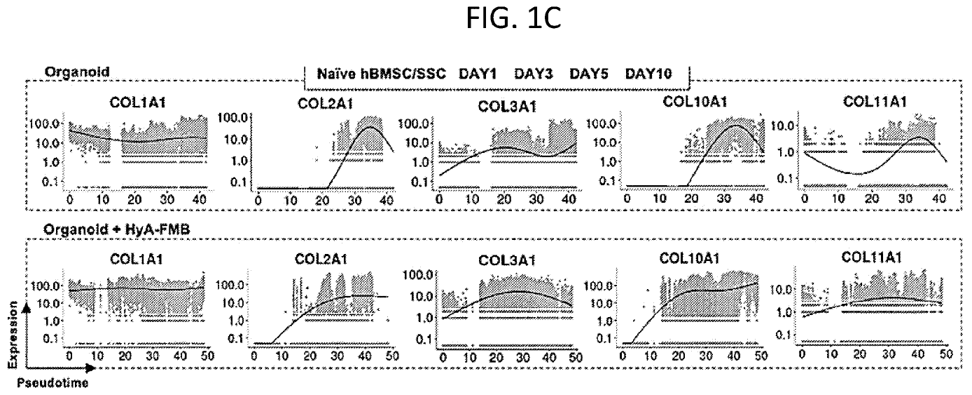

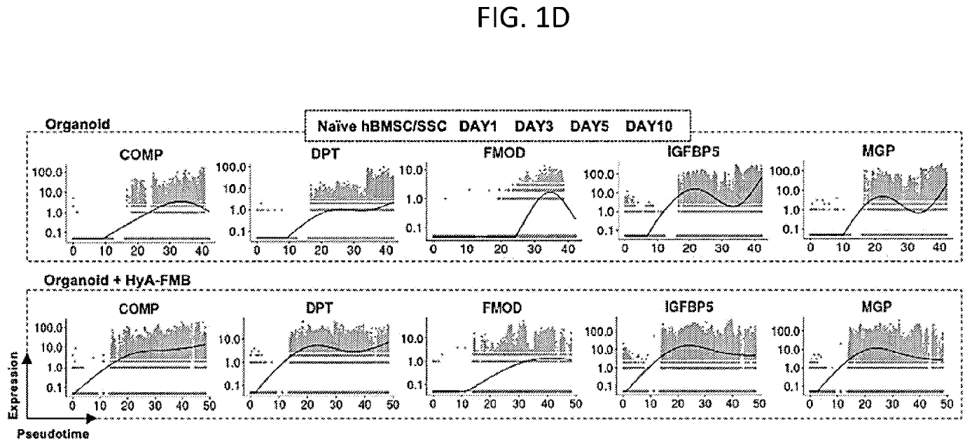

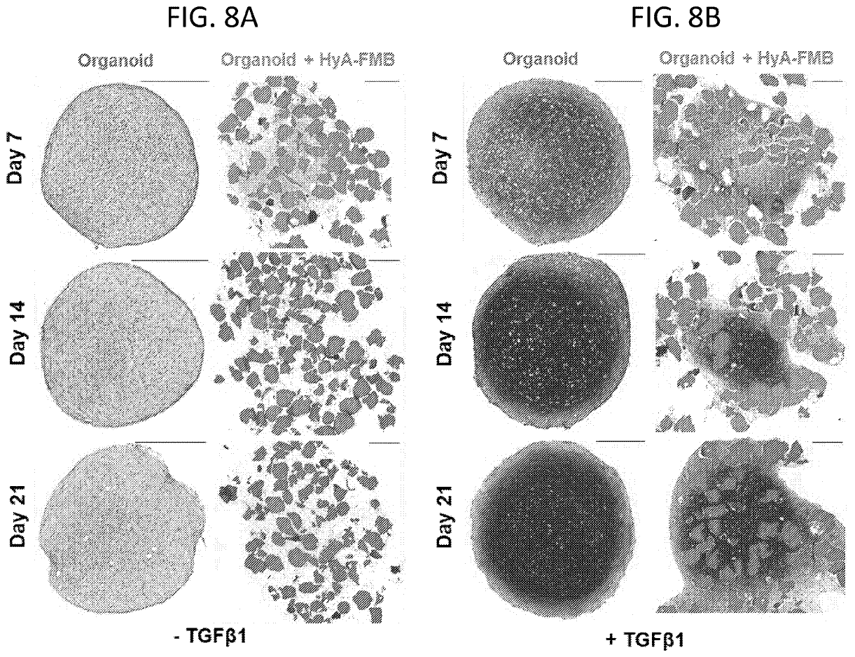

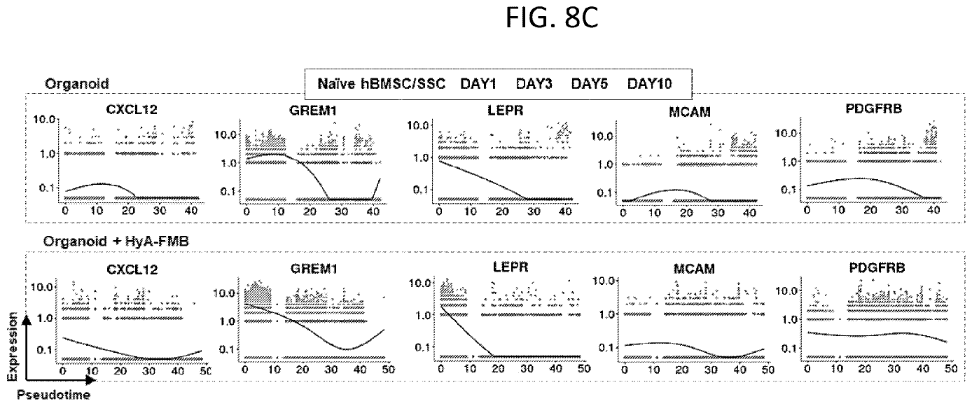

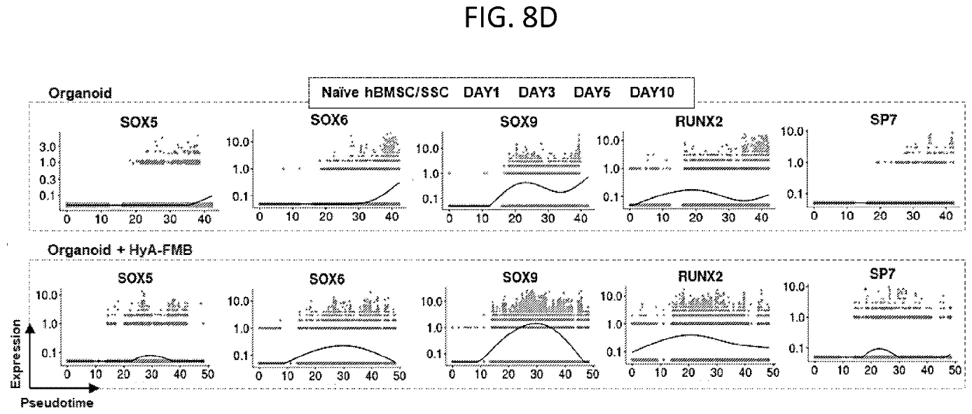

hBMSCs/SSCs differentiate into chondrocytes that form stable hyaline-like cartilage for up to 28 weeks when transplanted on HyA-FMBs (Kuznetsov et al., Stem Cells Translational Medicine, 26, 586-592, doi.org/10.1016/j.joca.2018.02.29, 2019). Therefore, we used HyA-FMBs as a model system to examine transcriptomic mechanisms that guide stable cartilage development and maintenance. Specifically, we cultured hBMSCs/SSCs attached to HyA-FMBs in chondrogenic differentiation medium and performed single-cell RNA sequencing (scRNA-seq) from digested organoids at days 1, 3, 5, and 10 (FIG. 1A). Over time, organoids formed a glycan-rich cartilage matrix indicated by Toluidine Blue staining (FIG. 1B) and required TGFβ1 supplementation for proper chondrogenic differentiation (FIGS. 8A-8B).

A total of 37,792 cells from control and 41,545 cells from HyA-FMB organoids were analyzed by scRNA-seq across all timepoints following ambient RNA and doublet removal (along with other quality control measures, see Methods), and Seurat integration was performed within control and HyA-FMB datasets (Stuart et al., Cell, 177(7), 1888-1902.e21, doi.org/10.1016/j.cell.2019.05.031, 2019). Pseudotime analysis across 10 days of chondrogenic differentiation revealed similarities in both organoid conditions, including decreased expression of primitive BMSC/SSC markers (i.e., CXCL12, GREM1, LEPR, MCAM, and PDGFRB) and induction of chondrogenic transcription factors (FIGS. 8C-8D). However, many differences were observed in the timing of genes coding for extracellular matrix proteins; i.e., genes coding for collagenous proteins (COL1A1, COL2A1, COL3A1, COL10A1, and COL11A1) and non-collagenous proteins (COMP, DPT, FMOD, IGFBP5, MGP) were induced earlier in HyA-FMB organoids (FIGS. 1C-1D). Thus, HyA-FMBs promote early induction of chondrogenic matrix genes, but are unable to prevent early expression of the hypertrophic marker, COL10A1, known to be induced with in vitro hBMSC/SSC chondrogenic differentiation (Pelttari et al., Arthritis and Rheumatism, 54(10), 3254-3266, doi.org/10.1002/art.22136, 2008).

### Example 2

### HyA-FMBs Suppress BMP Signaling Early in Chondrogenic Differentiation

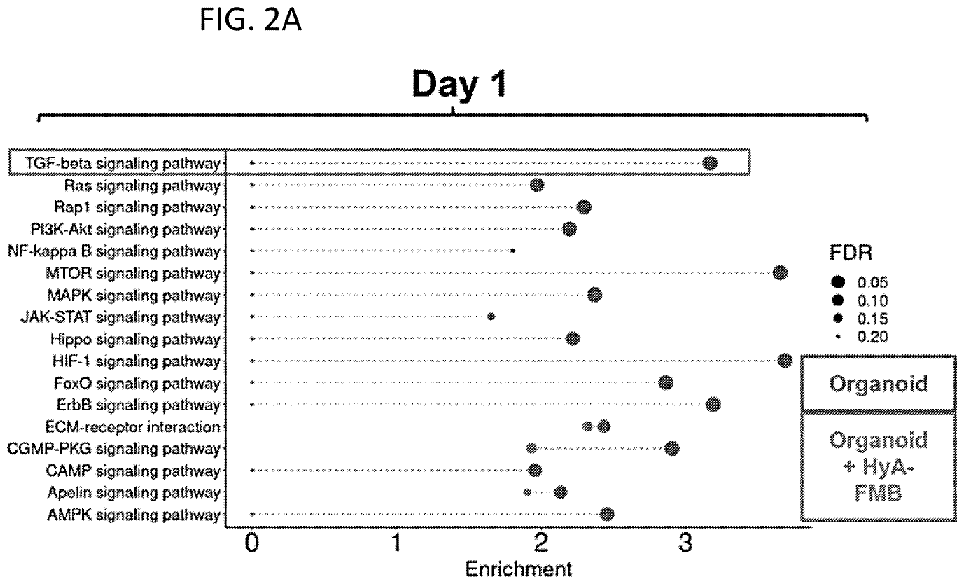

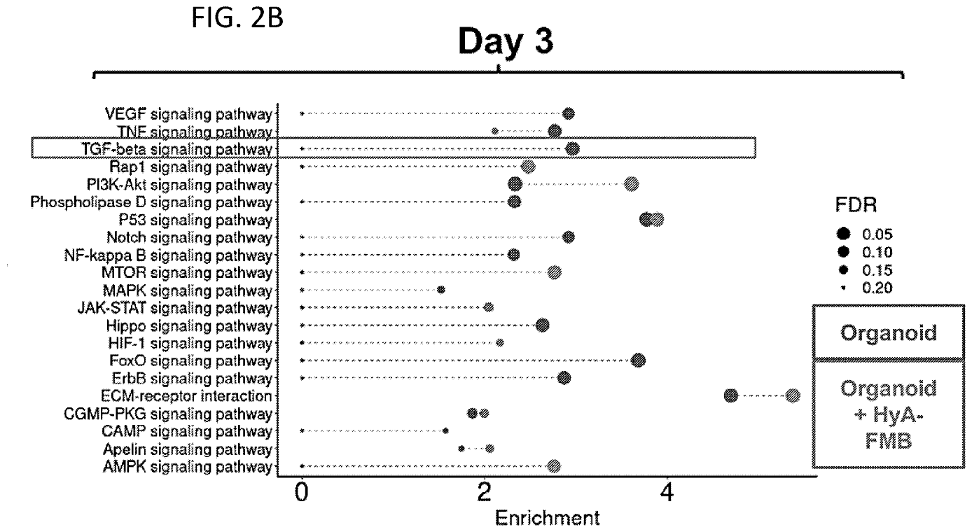

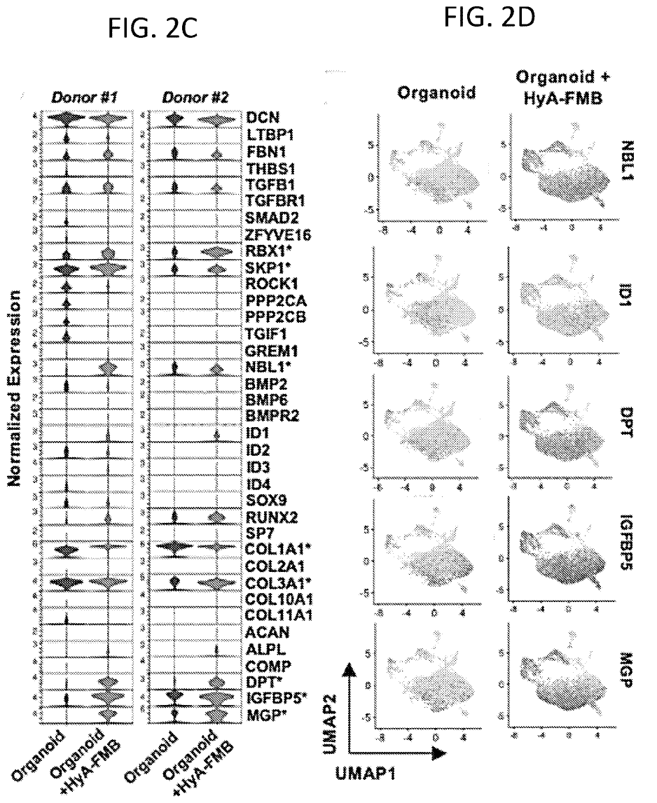

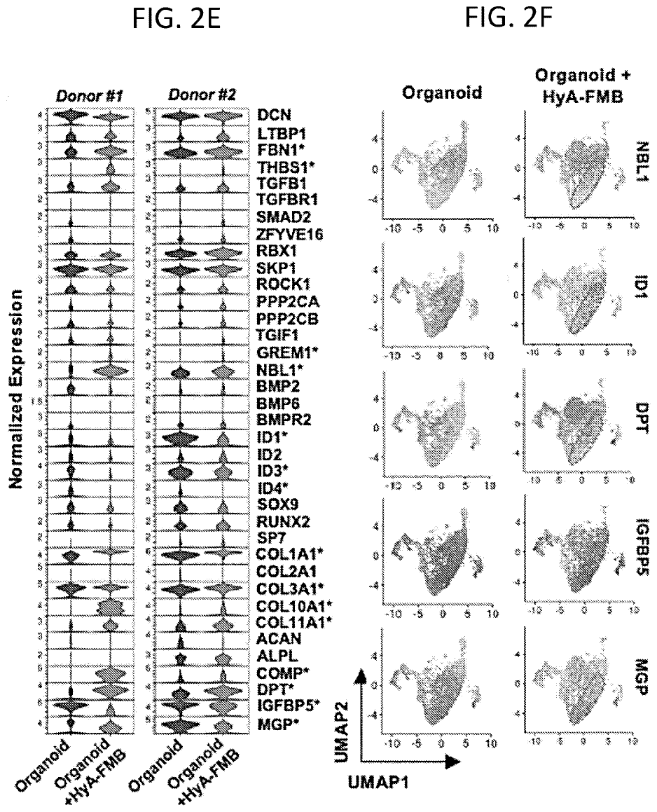

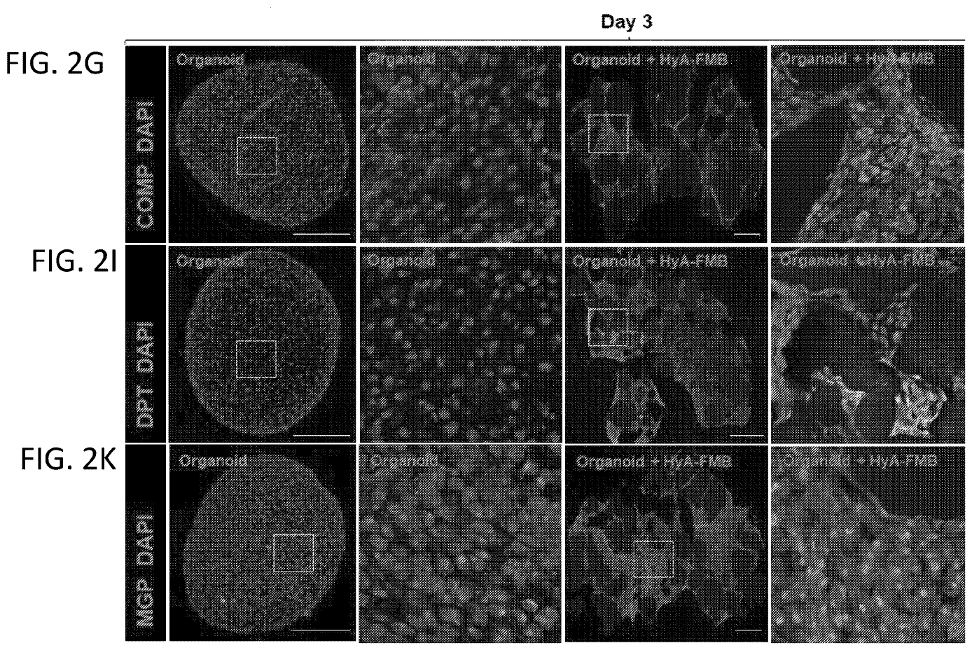

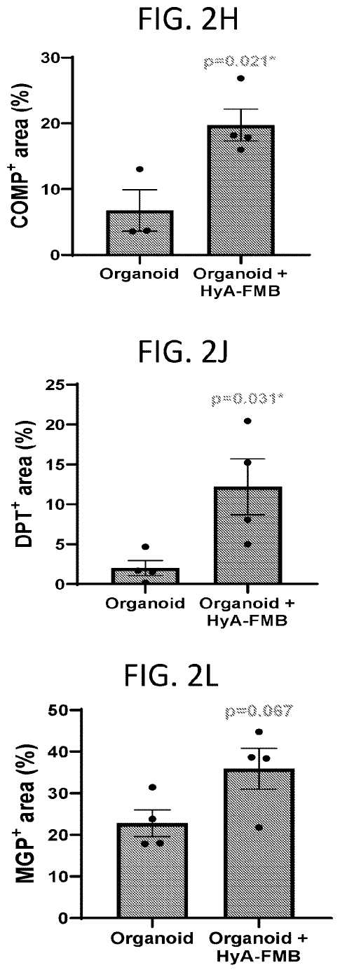

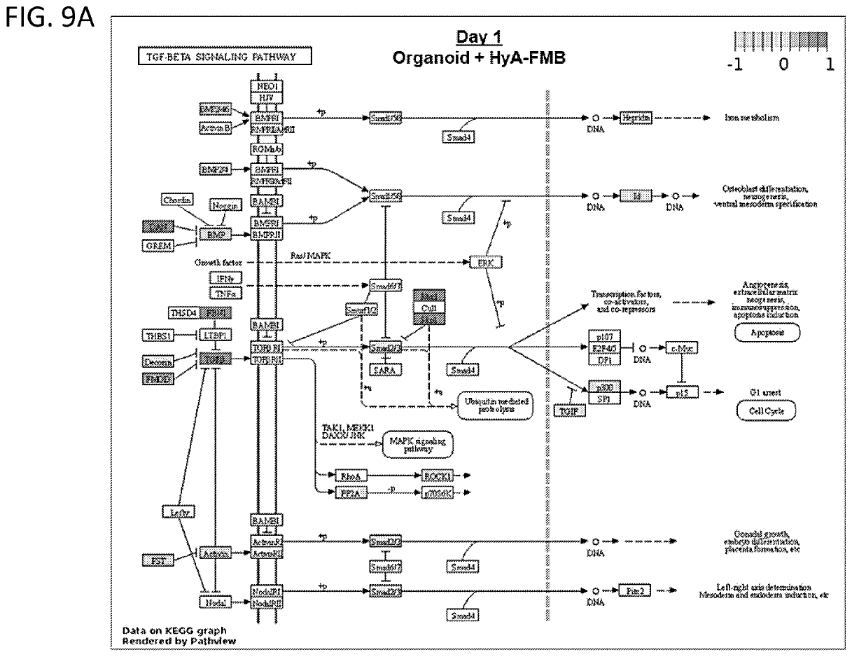

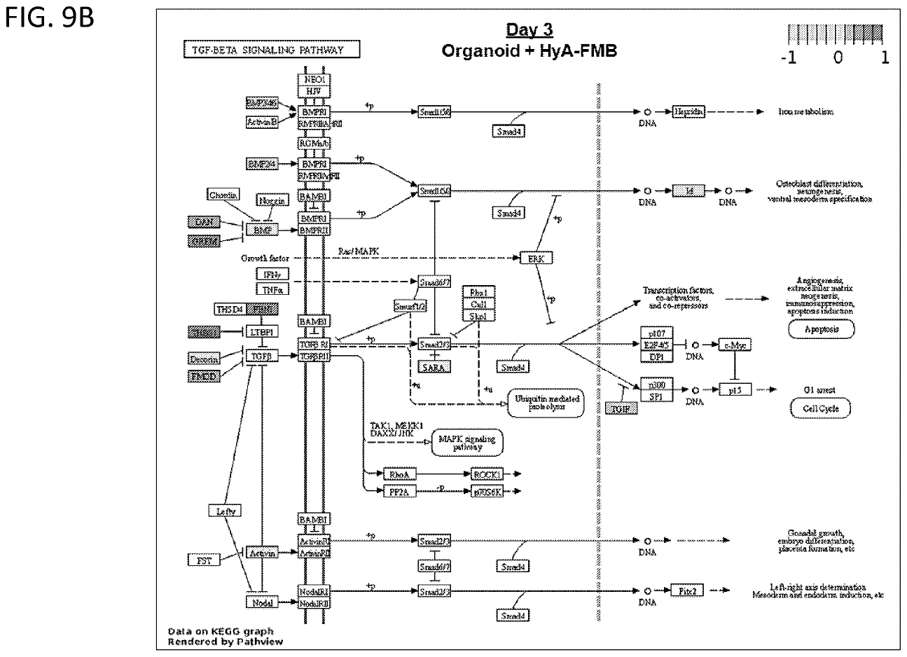

Since many differences associated with HyA-FMB organoids were observed early, we jointly analyzed control and HyA-FMB datasets at days 1 and 3 of chondrogenic differentiation from two hBMSC/SSC donors. For these studies, 26,307 control and 30,890 HyA-FMB single cell transcriptomes were processed, following quality control, and performed Seurat integration and normalization between control and HyA-FMB datasets for direct comparison. KEGG and pathview analysis of differentially expressed genes at days 1 and 3 revealed an enrichment of genes involved in TGFβ signaling in control, but not HyA-FMB organoids (FIGS. 2A-2B). At day 1, expression of TGFβ and BMP inhibitors (RBX1, SKP1, and NBL1) and extracellular matrix genes (COL1A1, COL3A1, DPT, IGFBP5, and MGP) were increased in HyA-FMB organoids (FIGS. 2C-2D and 9A). At day 3, BMP inhibitor expression (GREM1 and NBL1) remained increased with a corresponding decrease in ID1, ID3, and ID4 in HyA-FMB organoids, suggesting overall suppression of BMP signaling (FIGS. 2E-F and 9B). While expression of collagenous proteins remained higher in HyA-FMB organoids by day 3, expression of non-collagenous proteins was variable; i.e., COMP and DPT expression remained elevated by day 3, which was confirmed at the protein level by confocal microscopy, yet IGFBP5 and MGP expression decreased in HyA-FMB organoids compared with controls (FIGS. 2E-2L). Interestingly, while no chondrogenic cluster identities could be determined at days 1 and 3 of differentiation, IGFBP5 and MGP expression partitioned a subgroup of cells with lower levels of NBL1 and higher levels of ID) (FIG. 2F, dashed lines), prompting further examination of BMP signaling in MGP/IGFBP5-enriched cells during chondrogenic differentiation.

### Example 3

### HyA-FMBs Restore and Activate BMP Signaling in MGP/IGFBP5-Enriched Chondrogenic Cells

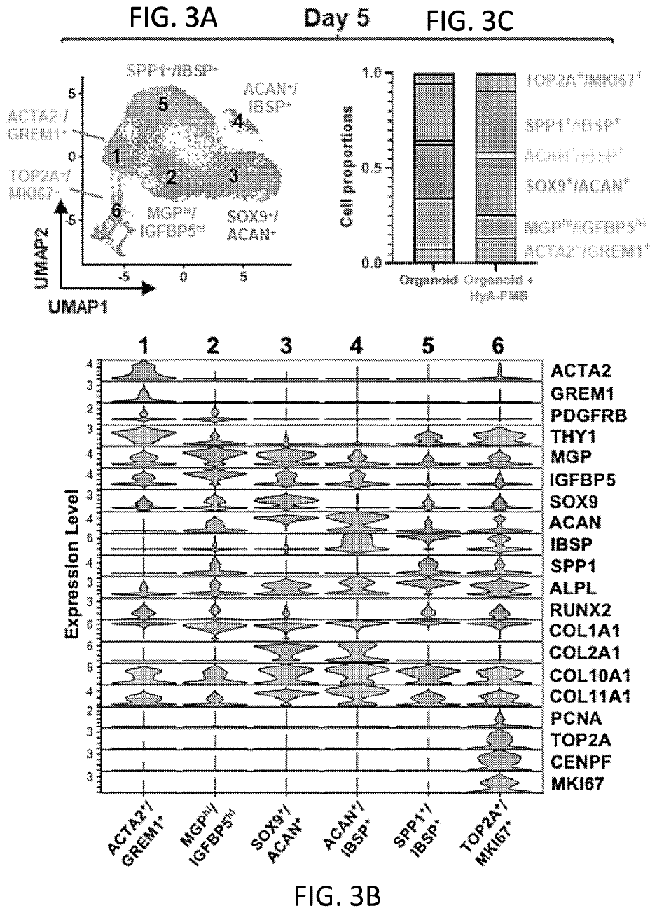

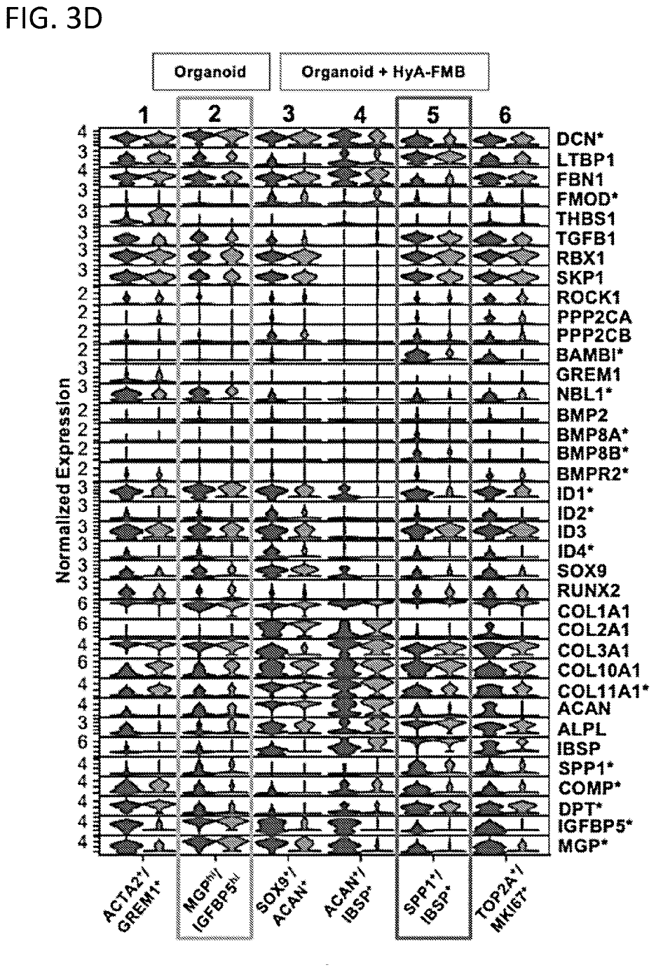

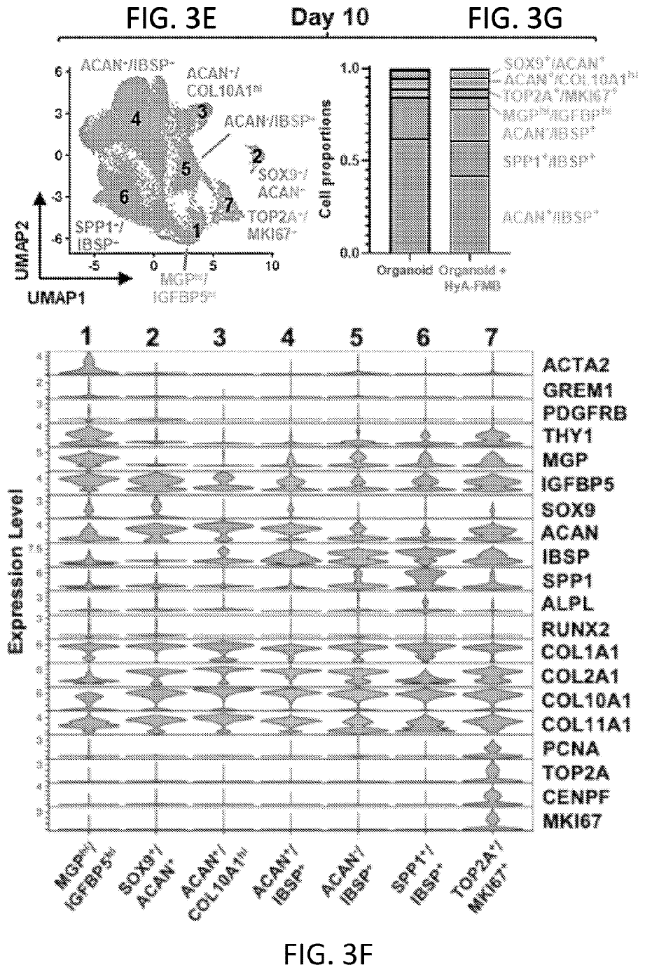

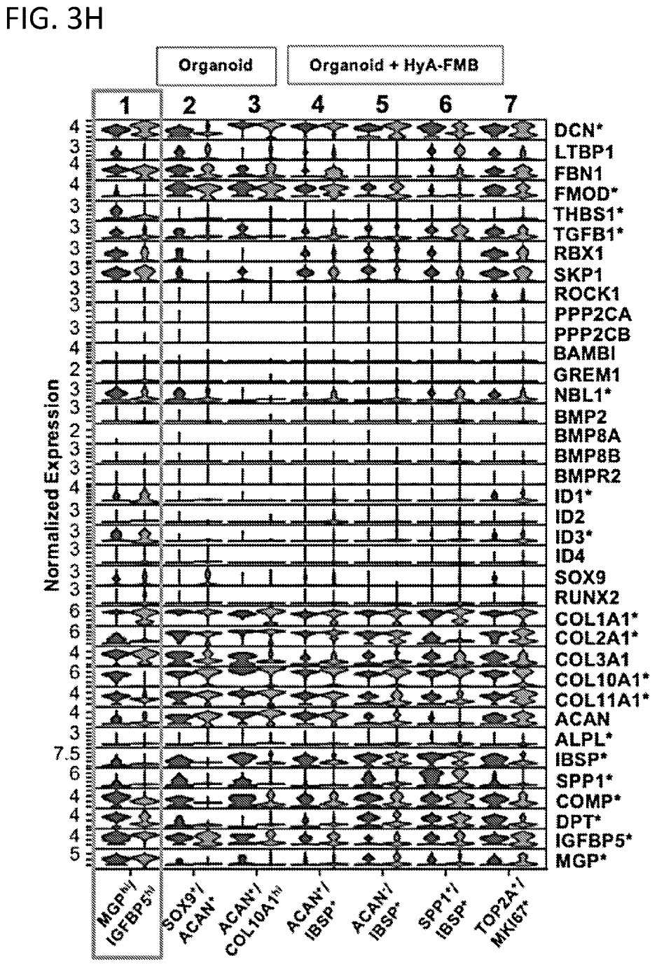

At day 5 of chondrogenic differentiation, more identifiable chondrogenic and osteogenic populations began to emerge in both control and HyA-FMB organoids. Specifically, cluster analysis of integrated day 5 datasets identified 6 populations: more primitive ACTA2⁺/GREM1⁺ cells, MGP^{hi}/IGFBP5^{hi} pre-chondrogenic cells, SOX9⁺/ACAN⁺ chondrogenic cells, ACAN⁺/IBSP⁺ chondro-osteogenic cells, SPP1⁺/IBSP⁺ osteogenic cells, and TOP2A⁺/MKI67⁺ cycling chondro-osteoprogenitor cells (FIGS. 3A-B). Cluster proportions were similar between control and HyA-FMB datasets, with the exception of the MGP^{hi}/IGFBP5^{hi} pre-chondrogenic cluster, which comprised 12% of HyA-FMB organoids compared with 27% of controls (FIG. 3C). While expression levels of IGFBP5 and MGP were globally decreased in HyA-FMB organoids at day 5, they were unchanged between groups within the MGP^{hi}/IGFBP5^{hi} pre-chondrogenic cluster, which exhibited similar expression of genes associated with TGFβ and BMP signaling (FIG. 3D, box). Many of the differences in BMP signaling genes arose in SPP1⁺/IBSP⁺ osteogenic cells; i.e, HyA-FMB organoids exhibited decreased levels of BMP8A, BMP8B, BMPR2, ID1, ID2, and ID4 compared with controls (FIG. 3D, box). These data suggest that HyA-FMBs decrease BMP signaling in SPP1⁺/IBSP⁺ osteogenic-committed cells, but restore BMP signaling in MGP^{hi}/IGFBP5^{hi} re-chondrogenic cells by day 5 of chondrogenic differentiation.

At day 10, IBSP⁺ cell populations comprised 90% of control organoids compared with 78% of HyA-FMB organoids, suggesting that the default osteogenic program in hBMSCs/SSCs was reduced in HyA-FMB organoids (FIGS. 3E-G). Proportions of MGP^{hi}/IGFBP5^{hi} pre-chondrogenic cells were similar between groups, yet IGFBP5 and MGP expression levels were increased in HyA-FMB organoids within this cluster (FIGS. 3G-H). Moreover, MGP/IGFBP5-enriched cells from HyA-FMB organoids exhibited a notable decrease in hypertrophic and osteogenic markers (COL1A1, COL10A1, ALPL, IBSP, and SPP1) and the non-collagenous markers, COMP and DPT (FIG. 311, red box). Expression of NBL1, ID1, and ID3 were almost exclusive to MGP^{hi}/IGFBP5^{hi} pre-chondrogenic cells and TOP2A⁺/MKI67⁺ cycling chondro-osteoprogenitor cells, with reduced NBL1 expression in MGP/IGFBP5-enriched cells (FIG. 3H, red box). Overall, these data suggest that HyA-FMBs restore and activate BMP signaling in MGP^{hi}/IGFBP5^{hi} enriched cells, which exhibit low expression of hypertrophic and osteogenic markers.

### Example 4

### Chronic Suppression of BMP-ID Signaling Promotes Osteogenic Gene Expression

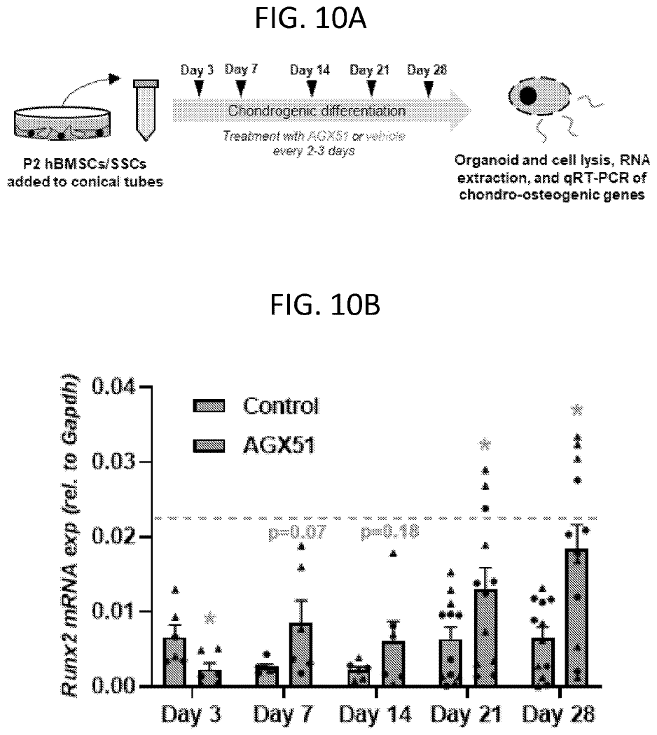

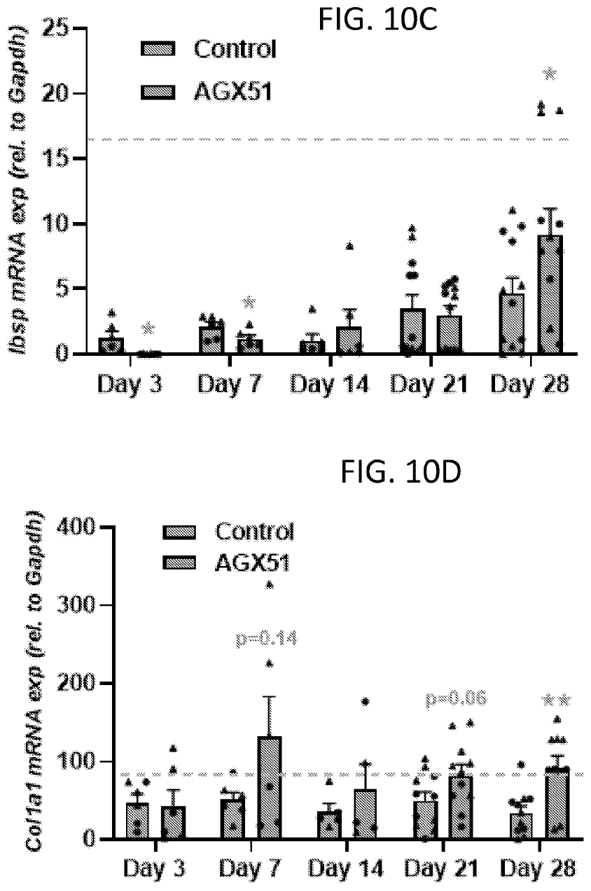

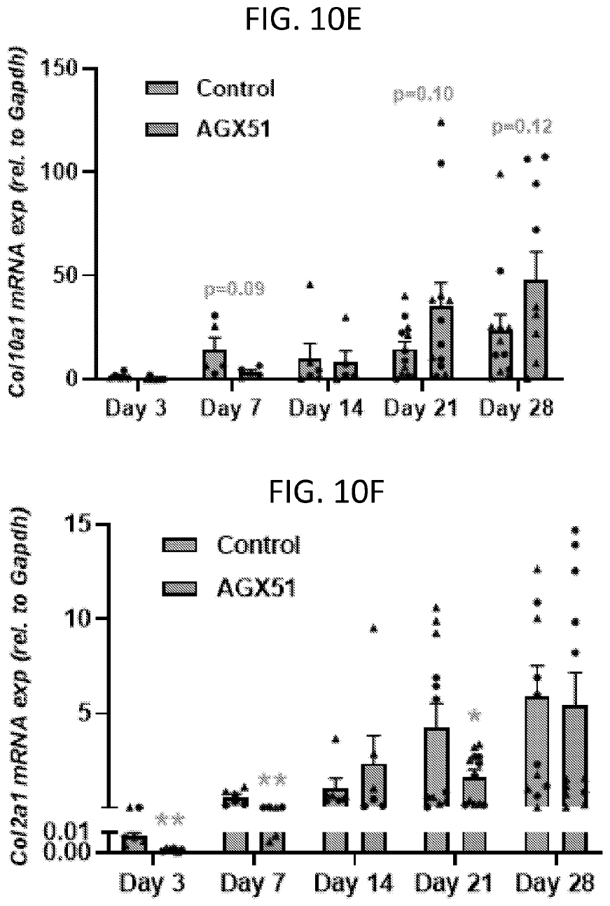

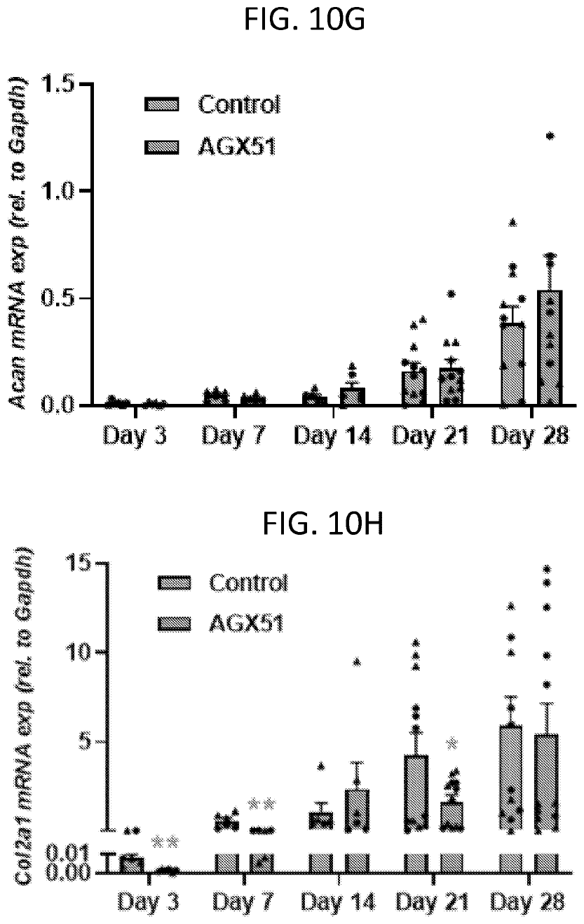

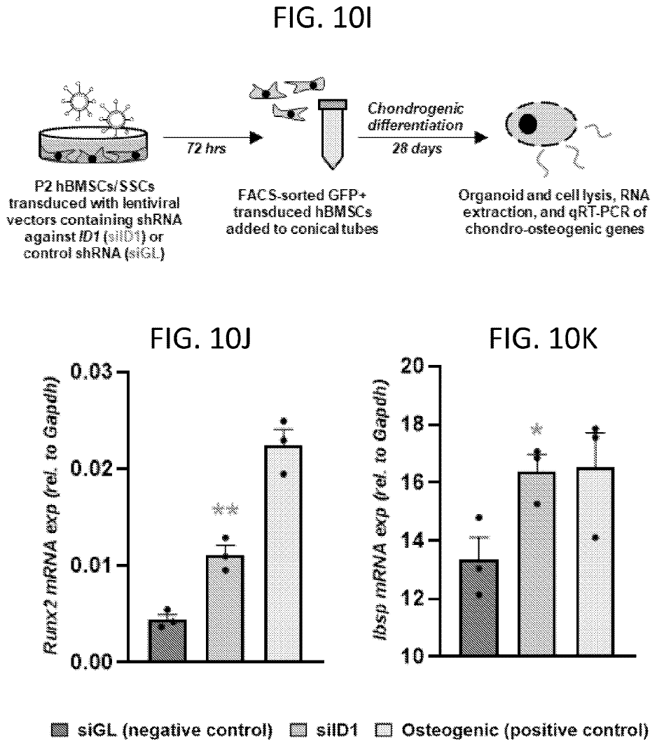

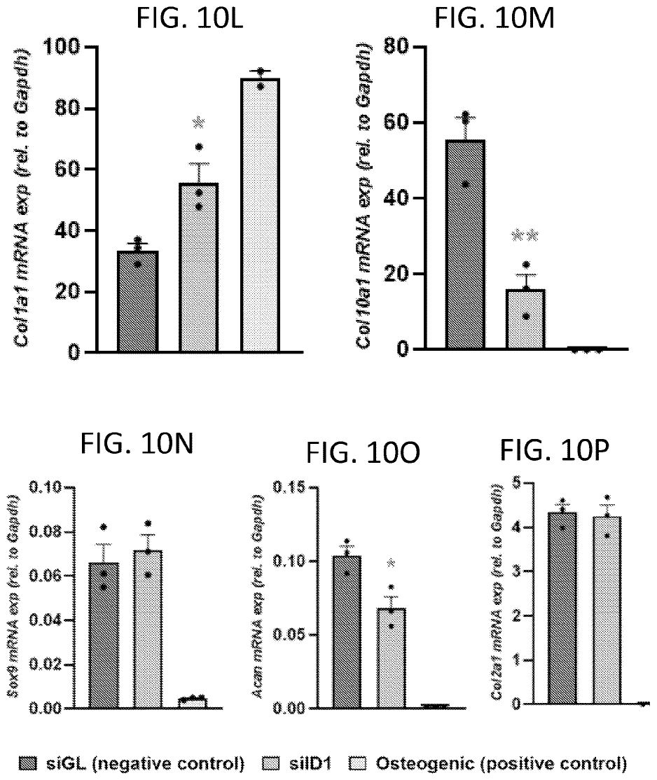

It was shown that HyA-FMBs initially decrease BMP signaling, then restore and activate BMP signaling in a chondrogenic population enriched for IGFBP5 and MGP. To functionally examine the BMP-ID signaling axis during chondrogenic differentiation, we treated control organoids from two hBMSC/SSC donors with AGX51 (a pan-ID inhibitor) for 28 days and monitored chondrogenic and osteogenic gene expression at days 3, 7, 14, 21, and 28 via qRT-PCR (FIG. 10A). ID blockade led to an initial decrease in RUNX2 and IBSP expression at days 3 and 7, followed by an increase in RUNX2, IBSP, and COL1A1 expression from days 21-28 (FIGS. 10B-10D). COL10A1 expression showed a similar trend (Figure S3E). Further, chondrogenic gene expression (SOX9, ACAN, and COL2A1) was initially decreased, but stabilized with ID blockade (FIGS. 10F-10H). These data suggest that the BMP-ID signaling axis promotes chondro-osteogenic transcription early, but suppresses osteogenic and hypertrophic expression later in chondrogenic differentiation. To specifically test ID1 in chondro-osteogenic commitment, ID1 expression was knocked down in passage 2 hBMSCs/SSCs using lentiviral vectors containing shRNA against ID1, and differentiated transduced organoids in chondrogenic medium for 28 days followed by qRT-PCR analysis (FIG. 10I). Knockdown of ID1 produced a similar increase in RUNX2, IBSP, and COL1A1 expression after 28 days of chondrogenic differentiation (FIGS. 10J-10L); contrastingly, COL10A1 expression was decreased following ID1 knockdown (FIG. 10M). Further, SOX9 and COL2A1 expression were unaffected, while ACAN expression decreased, following chronic ID) suppression (FIGS. 10N-10P). Overall, these results suggest that chronic suppression BMP-ID1 signaling during chondrogenic differentiation promotes osteogenesis—evidenced by increased RUNX2, IBSP, and COL1A1 expression—yet the discordance of COL10A1 and ACAN expression between experiments suggests a potential role of other ID genes in regulating chondro-osteogenic expression.

### Example 5

### BMP-ID Signaling is Increased in Stable, Hyaline-Like Cartilage Upon Ectopic Transplantation

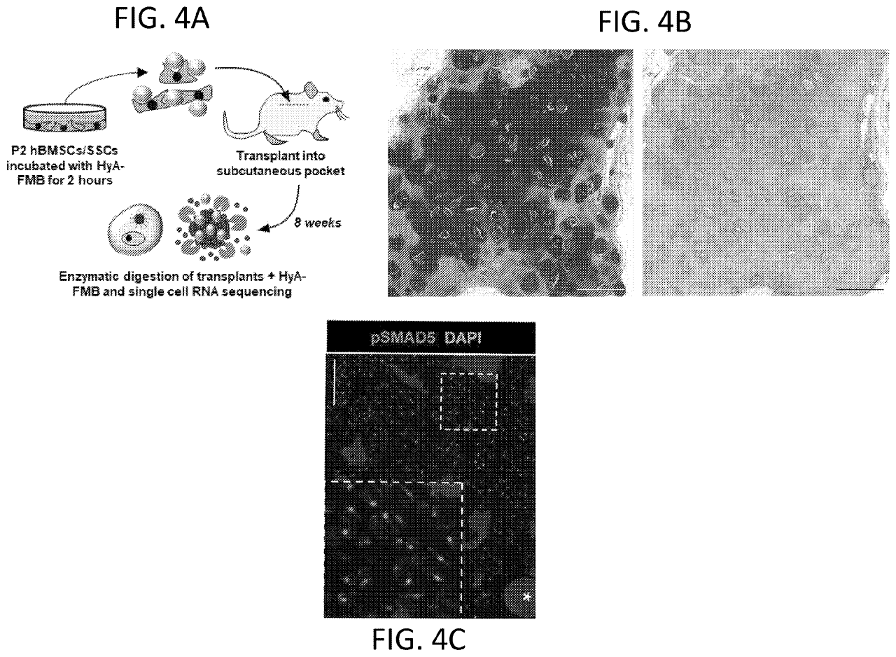

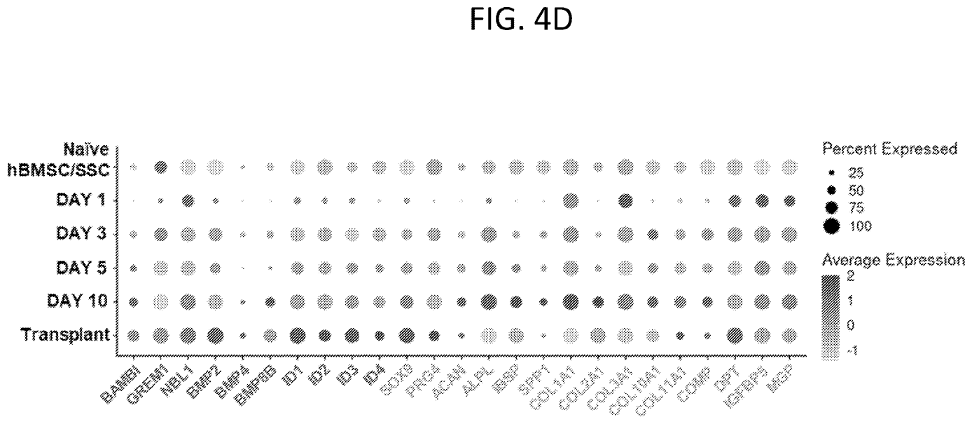

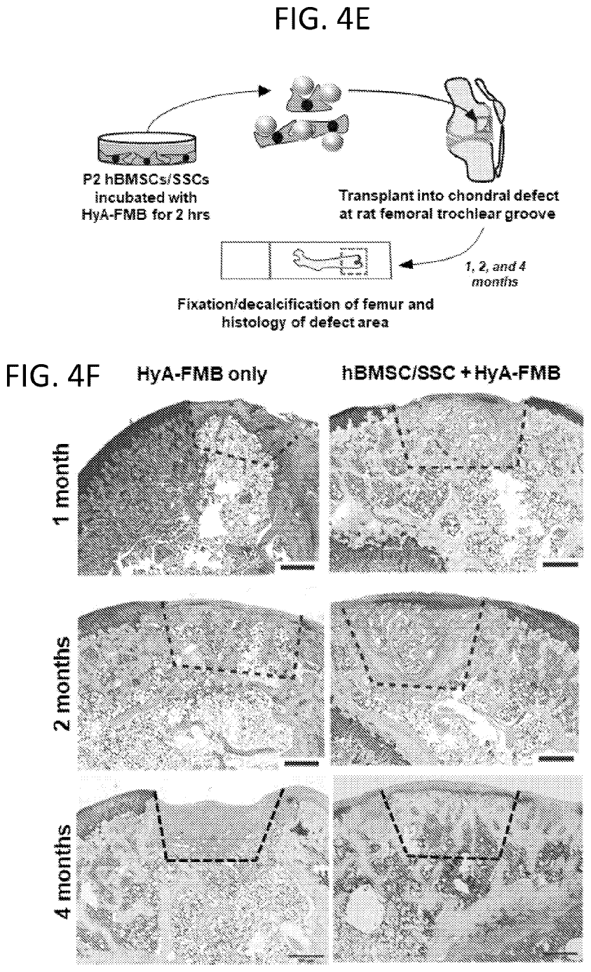

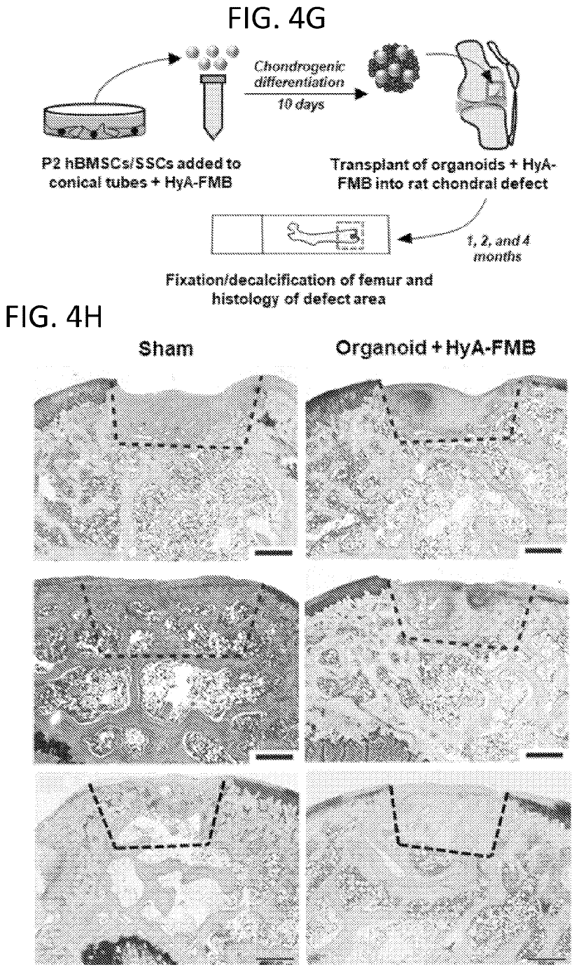

Since chronic BMP-ID1 suppression promotes osteogenic expression, we wondered whether activating genes in the BMP-ID axis would be upregulated in stable, transplanted cartilage. To test this, hBMSCs/SSCs attached to HyA-FMBs were transplanted ectopically into immunocompromised NSG mice and performed scRNA-seq of digested cartilage tissues 8 weeks-post-transplant (FIG. 4A). Ectopic HyA-FMB transplants formed a hyaline-like cartilage matrix by 8 weeks, indicated by Toluidine Blue and H&E staining (FIG. 4B top) and H&E staining (FIG. 4B bottom). pSMAD5, indicative of BMP signaling is found in the newly formed cartilage (FIG. 4C). (FIG. 4B-C—* indicate HyA-FMBs). Integration and normalization of in vitro and in vivo HyA-FMB datasets were performed for cross-comparison. Transplanted tissues exhibited increased levels of PRG4 and decreased levels of ALPL, IBSP, SPP1, COL1A1, and COL10A1 compared with in vitro culture, confirming a more hyaline-like phenotype in the in vivo model (FIG. 4D). Moreover, BMP2, ID1, ID2, ID3, and ID4 were increased in transplanted tissues, suggesting a potential role of BMP activation in maintaining stable cartilage (FIG. 4D). IGFBP5 and MGP expression were highest at day 1 of chondrogenic differentiation and maintained lower but stable levels in more mature tissues (FIG. 4D). These data confirm the activation of BMP-ID signaling genes in mature, stable cartilage.

### Example 6

Rat Chondral Transplantation of hBMSC/SSC/HyA-FMB Constructs Yields Suboptimal Chondrogenesis

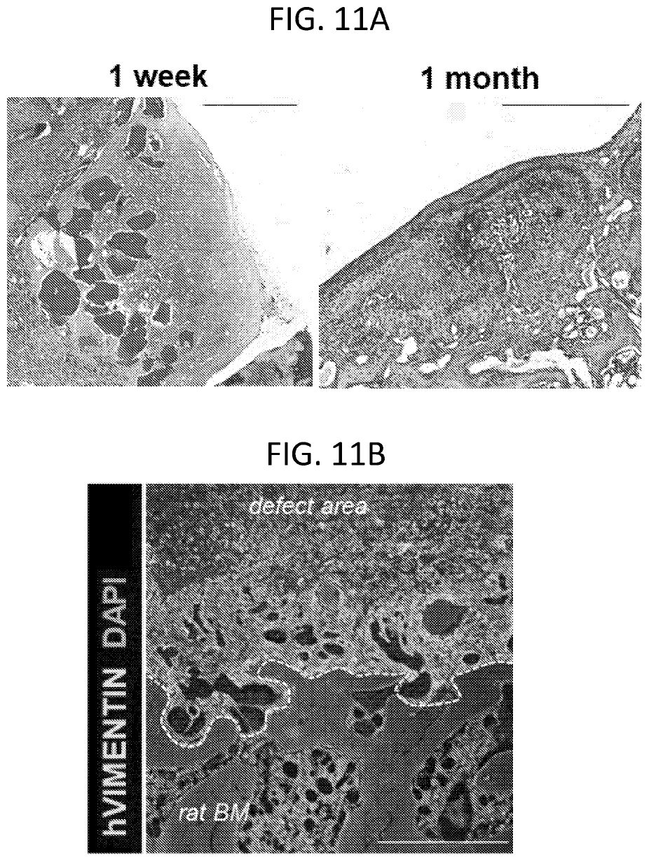

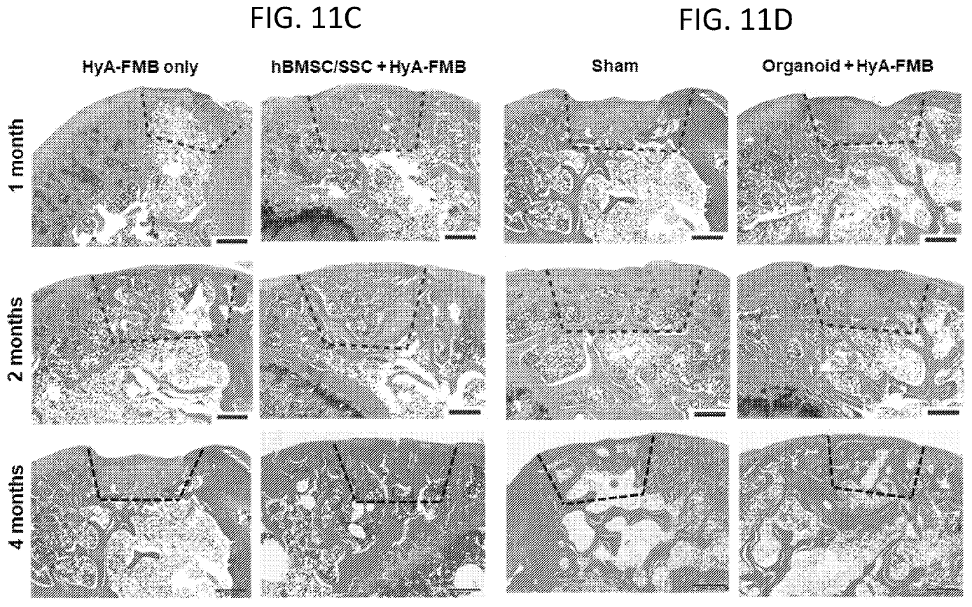

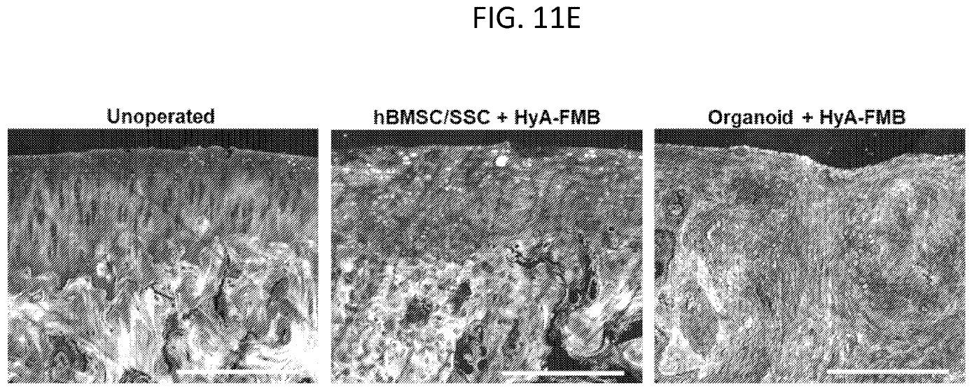

To test the stability of cartilage tissues formed by hBMSCs/SSCs attached to HyA-FMBs in a clinically-relevant chondral defect model, hBMSCs/SSCs attached to HyA-FMBs were transplanted into a 2 mm-defect at the femoral trochlear groove in immunodeficient SRG rats, followed by histology of the defect site at 1, 2, and 4 months-post-transplant (FIG. 4E). Unexpectedly, transplanted HyA-FMBs dissolved after 1 month at the defect site, which was identified by hVIMENTIN staining (FIGS. 11A-11B). Toluidine Blue-stained cartilage matrix was evident on the joint surface in the defect site 1 month- and 2 months-post-transplant, yet surface Toluidine Blue staining was less evident by 4 months, with organization of subchondral bone and marrow in the defect site (FIGS. 4F and 11C). It was determined whether a 10-day chondrogenic induction period in vitro would enhance the stability of transplanted HyA-FMBs, and transplanted pre-formed HyA-FMB organoids into the defect site of SRG rats (FIG. 4G). Toluidine blue and H&E staining revealed a similar dissolution of HyA-FMBs by 1 month, vascular invasion by 2 months, and replacement by bone at 4 months (FIGS. 4H and 11D). Second harmonic generation imaging of defect sites revealed collagen organization of transplanted hBMSCs/SSCs/HyA-FMB constructs comparable to healthy tissues, whereas pre-differentiated HyA-FMB organoids formed a more haphazard matrix (FIG. 11E). While these data show that hBMSC/SSC/HyA-FMB constructs regenerate subchondral bone following chondral injury, the dissolution of HyA-FMBs and propensity of bipotent hBMSCs/SSCs to form fibrocartilage and bone in the absence of a scaffold (Pelttari et al., Arthritis and Rheumatism, 54(10), 3254-3266, doi.org/10.1002/art.22136, 2006; Serafini et al., Stem Cell Research, 12(3), 659-672, doi.org/10.1016/j.scr.2014.01.006, 2014) restricts the translational potential of these cells in regenerating surface cartilage. Different strategies need to be identified to prevent hypertrophy of hBMSCs/SSCs.

### Example 7

A Novel Serum-Free Chondrogenic Differentiation Strategy Beginning with Sclerotome Induction

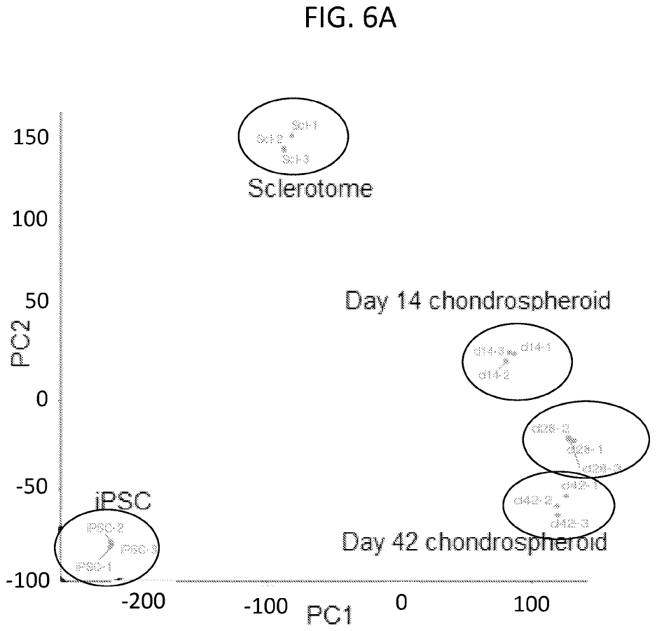

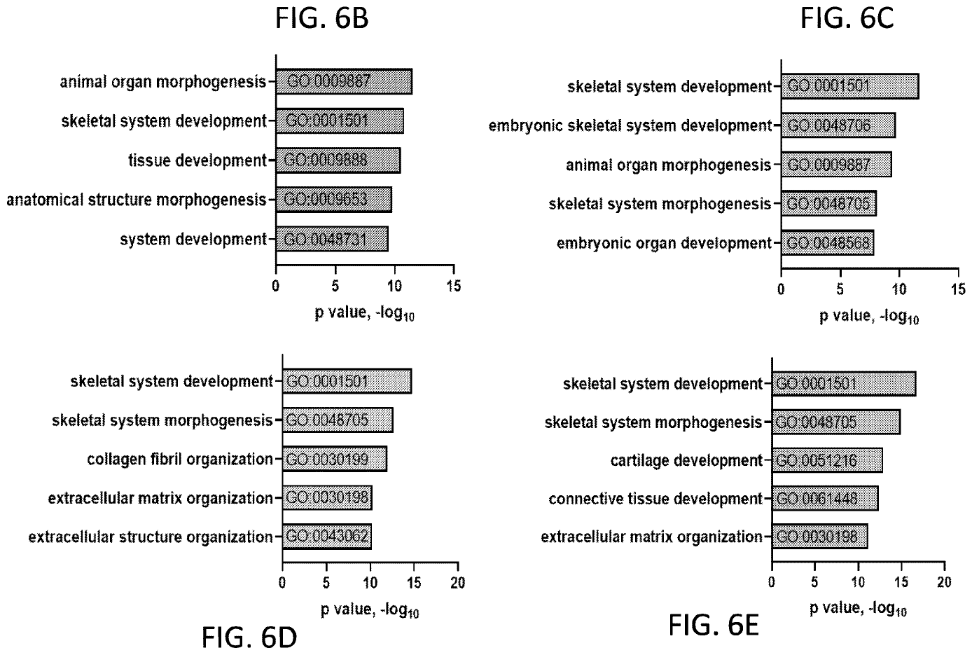

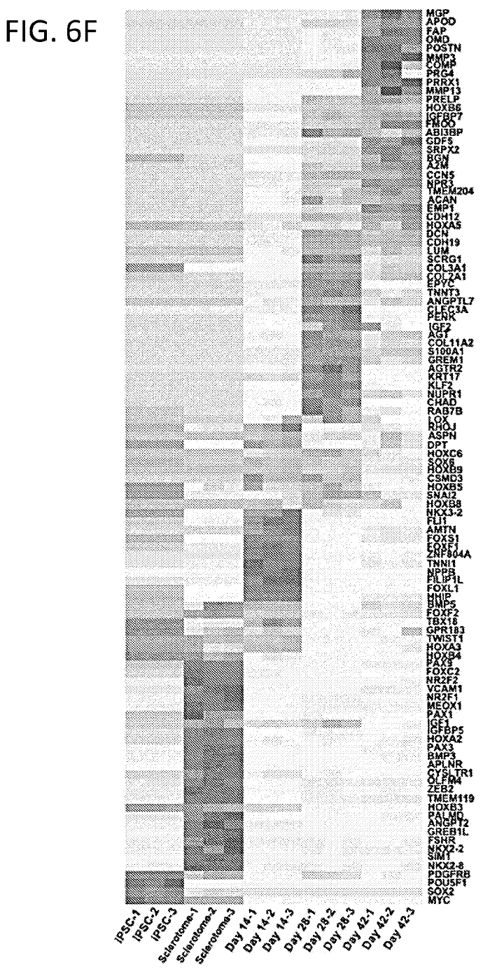

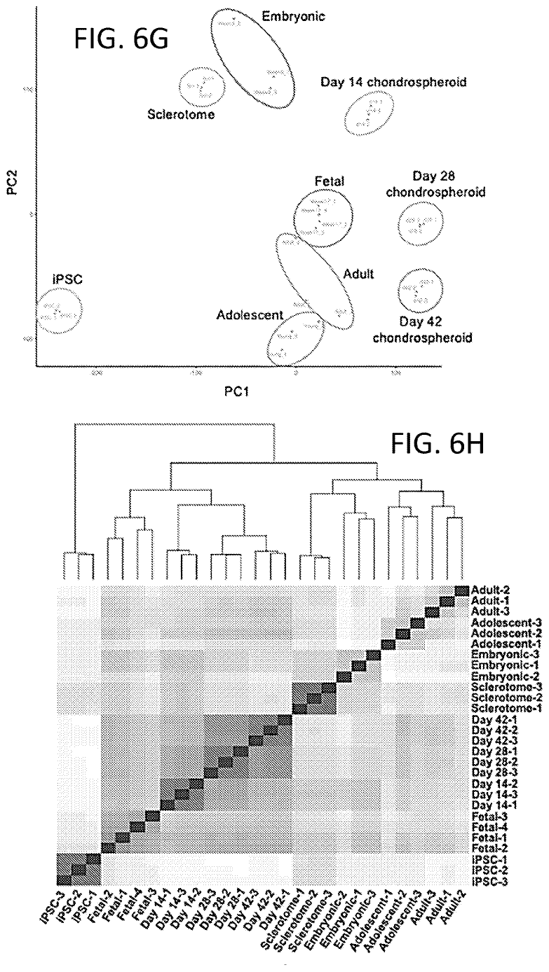

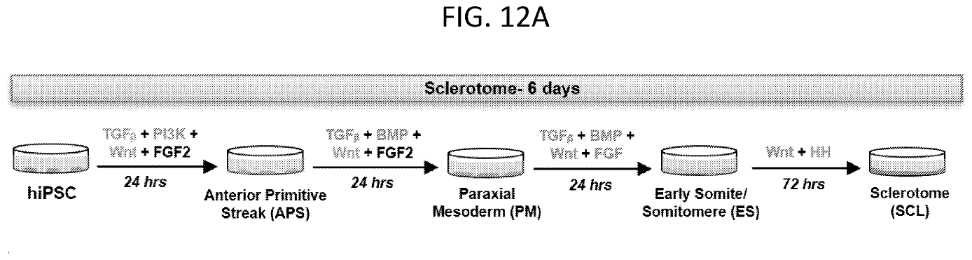

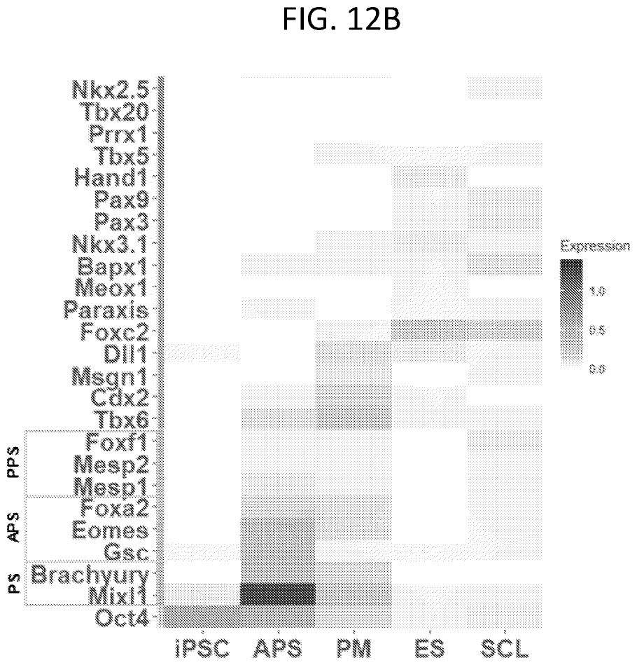

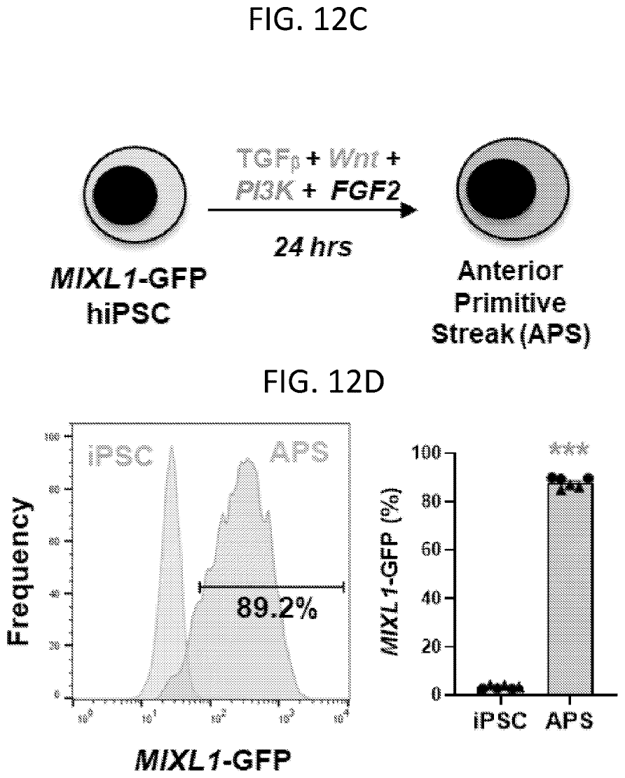

While hBMSCs/SSCs follow a default program leading to endochondral ossification in the absence of HyA-FMBs in vivo, reprogrammed cells like hiPSCs may escape this default program. Therefore, utilizing mechanistic knowledge from the HyA-FMB model system, a serum-free hiPSC differentiation protocol was established that employs BMP inhibition during initial differentiation, followed by BMP activation in pre-chondrogenic cells, with the goal of obtaining a stable chondrocyte-like cell for clinical translation. First, an initial 6-day sclerotome induction period first proposed in human ESCs (Loh et al., Cell, 166(2), 451-467, doi.org/10.1016/j.cell.2016.06.011, 2016) was utilized (FIGS. 5A and 12A, bar). Inhibiting PI3K and activating TGFβ, Wnt, and FGF signaling in hiPSCs for 24 hours resulted in the induction of primitive streak markers (BRACHYURY and MIXL1) with higher expression of anterior (GSC, EOMES, FOXA2) compared with posterior primitive streak markers (MESP1, MESP2, FOXF1) (FIG. 12B, bar). For confirmation, this same treatment in MIXL1-GFP hiPSCs produced ~90% GFP expression within 24 hours of differentiation (FIGS. 12C-12D). TGFβ and BMP pathway inhibition of anterior primitive streak cells for 48 hours, with concomitant targeting of Wnt and FGF pathways (see FIG. 12A), induced paraxial mesoderm markers (TBX6, CDX2, MSGN1, and DLL1) followed by genes associated with somitogenesis (FIG. 12B). Finally, inhibiting Wnt and activating Hedgehog pathways in somitomere cells induced sclerotome markers (FOXC2, BAPX1, and PAX9), along with minimal expression of lateral or cardiac mesoderm markers (FIG. 12B). RNA sequencing of sclerotome cells confirmed induction of FOXC2, PAX1, and PAX9, as well as IGFBP5 (see FIG. 6F). Thus, the sclerotome induction protocol characterized in hESCs (Loh et al., supra 2016) also produces efficient sclerotome differentiation in hiPSCs.

### Example 8

### BMP Activation in SOX9⁺ Purified Pre-Chondrogenic Cells Promotes Stable Chondrogenesis In Vitro

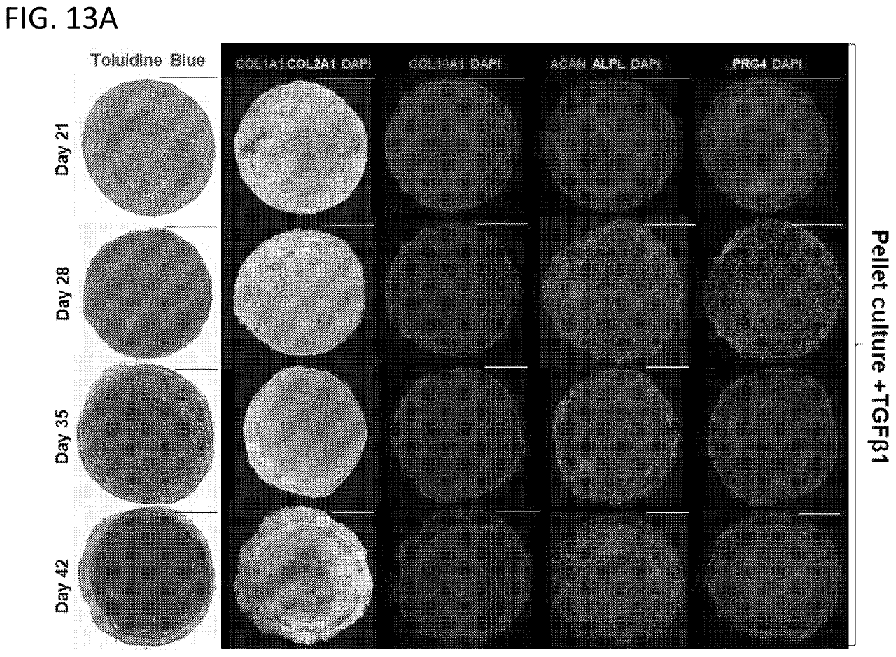

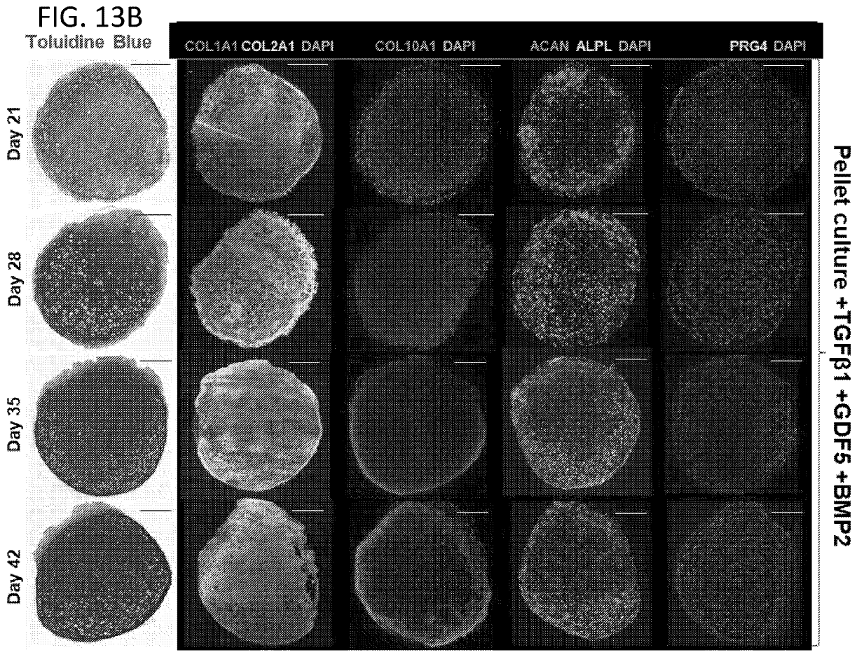

Since sclerotome cells are enriched for IGFBP5, BMP signaling was activated in a chondrogenic induction protocol (FIG. 5A, bar). As a negative control, supplementation of sclerotome-derived pellet cultures with TGFβ1 alone led to stable COL1A1 and COL2A1 expression across 42 days, with sporadic ACAN and minimal PRG4 expression (FIGS. 5B and 13A); these data confirm incomplete chondrogenesis in cultures treated with only TGFβ ligands (De Kinderen et al., supra, 2022) (see FIG. 3). Sclerotome-derived pellet cultures were supplemented with TGFβ1 and the BMP signaling activators, BMP-2 and GDF-5, which produced strong COL2A1 and ACAN expression, but a progressive increase in COL10A1 and ALPL across 42 days, suggesting improved chondrogenic differentiation but increased hypertrophy of hiPSC-derived chondroprogenitors (FIGS. 5B and 13B). Since BMP-ID signaling activates chondrogenic and osteogenic transcription early in hBMSCs/SSCs and may nurture distinct osteogenic and chondrogenic microenvironments (see FIG. 3 and FIGS. 10A-10P), it was hypothesized that BMP activation in a purified pre-chondrogenic subset of cells would produce a more homogenous and stable chondrogenic phenotype. To identify a pre-chondrogenic subset of cells, monolayer sclerotome cultures derived from SOX9-mCherry hiPSCs were treated with chondrogenic medium supplemented with TGFβ1, BMP-2, and GDF-5. Incucyte imaging revealed a progressive increase in SOX9-mCherry area and intensity in sclerotome cultures across 10 days of chondrogenic induction (FIG. 5C). Interestingly, groups of SOX9-mCherry⁺ cells began to condense after 6 days of chondrogenic induction in sclerotome cultures, and form loosely attached SOX9-mCherry^{bright} aggregates after 10 days while surrounding adherent cells showed little to no SOX9-mCherry expression (FIG. 5D). Therefore, these SOX9-mCherry^{bright} aggregates—hereafter termed chondrospheroids—were transferred to suspension cultures and BMP activation was continued selectively in this subgroup of pre-chondrogenic cells. Chondrospheroids transiently expressed COL1A1, followed by expression of strong COL2A1, ACAN, and PRG4 expression, starting from the exterior and moving inwards, with little to no COL10A1 expression (FIG. 5E); chondrospheroid formation and expression patterns were confirmed in hiPSCs without the SOX9 reporter and quantified over time (see FIG. 5B). Overall, non-selective BMP activation in all sclerotome cells induced hypertrophic chondrogenesis, yet selective BMP activation in a SOX9⁺ chondrogenic subset induced stable chondrogenesis in vitro.

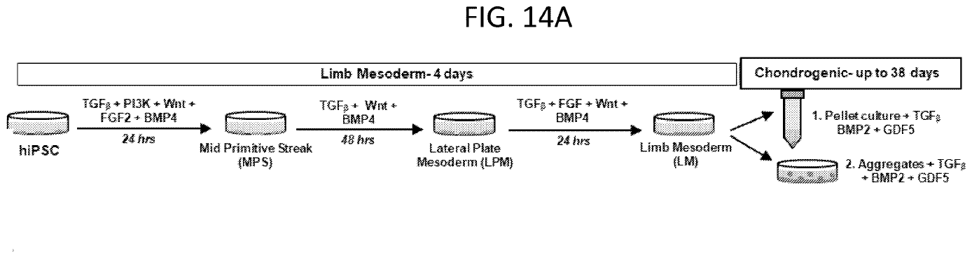

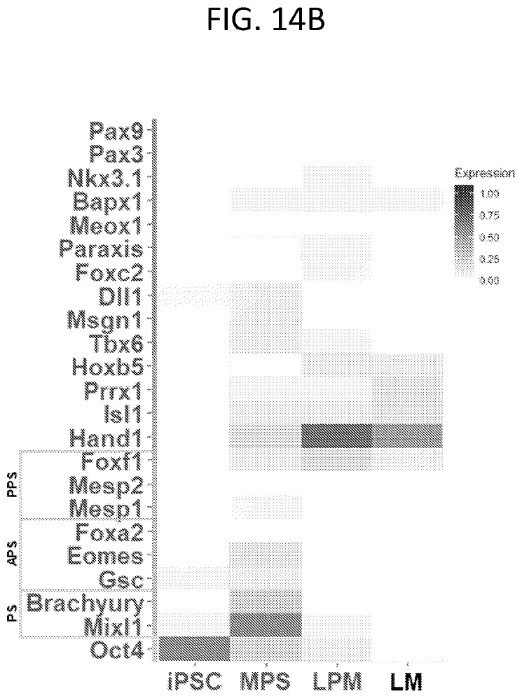

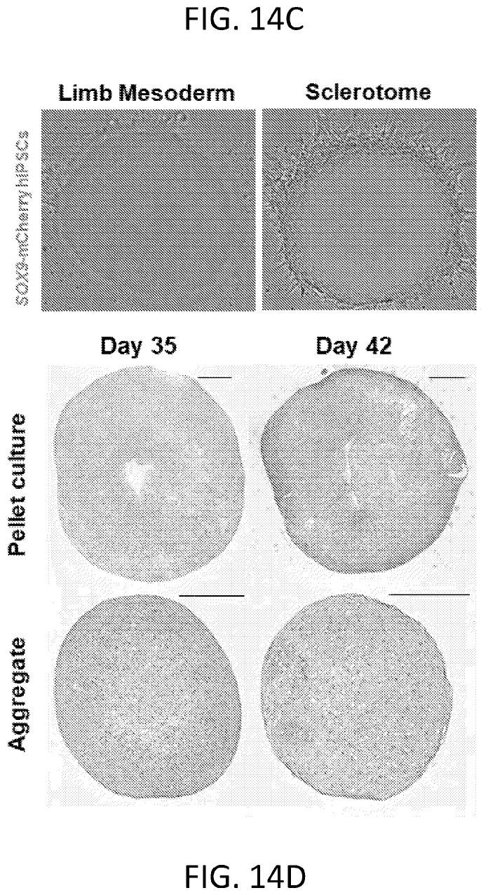

A limb (lateral plate) mesoderm induction period was also employed in hiPSCs prior to chondrogenic differentiation to examine chondrogenesis with chronic BMP activation. Specifically, supplementation of hiPSCs for 4 days with BMP-4 and compounds that target TGFβ, Wnt, and FGF signaling pathways led to expression markers of mid primitive streak, lateral plate mesoderm, and limb mesoderm (FIGS. 14A-14B), confirming previous studies (Loh et al., supra, 2016). Adding TGFβ1, BMP-2, and GDF-5 to limb mesoderm monolayer cultures led to the formation of loosely attached aggregates, similar to sclerotome cultures; however, SOX9-mCherry expression was significantly lower in limb mesoderm-derived aggregates (FIG. 14C). Moreover, little to no cartilage matrix was formed in limb mesoderm-derived pellet cultures and aggregates, indicated by Toluidine Blue staining at days 35 and 42 of differentiation (FIG. 14D), emphasizing the requirement of initial sclerotome induction (which inhibits BMP signaling) in efficient hiPSC chondrogenesis.

### Example 9

### Chondrospheroid Transcriptomes Show Efficient Chondrogenic Differentiation

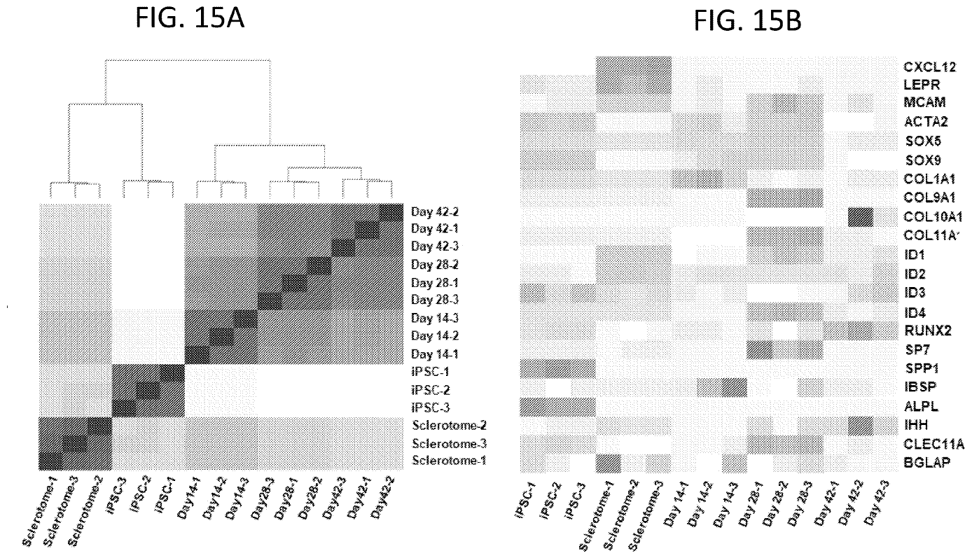

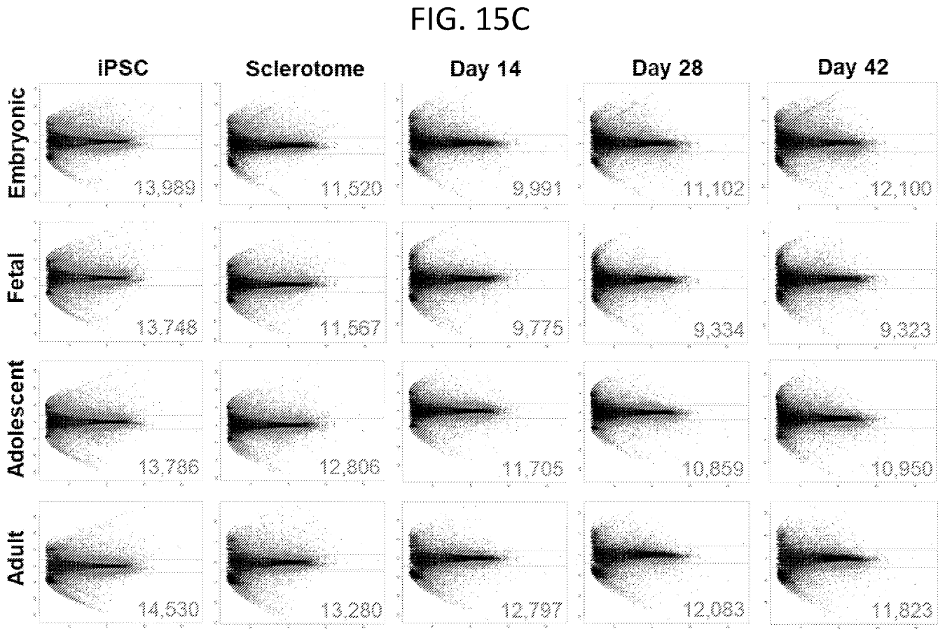

To confirm sclerotome and chondrogenic differentiation at the transcriptional level, RNA sequencing of sclerotome cells and sclerotome-derived chondrospheroids was performed at days 14, 28, and 42 of differentiation. Principal component analysis (PCA) (FIG. 6A) and hierarchical clustering (FIG. 15A) showed similarities among day 14, 28, and 42 chondrospheroids, but differences between chondrospheroids and sclerotome cells. Gene ontology (GO) analysis of sclerotome cells enriched for genes associated with tissue development and organ morphogenesis (FIG. 6B). with high expression of FOXC2, IGFBP5, MEOX1, PAX1, and PAX9 (FIG. 6F). Additionally, many primitive BMSC/SSC markers were highly expressed in sclerotome cells—such as CXCL12, LEPR, and PDGFRB—while others peaked later in differentiation, including MCAM and GREM1 (FIGS. 6F and 15B). Several HOX family genes were differentially expressed early in chondrogenic differentiation, as day 14 chondrospheroids were enriched for genes associated with embryonic skeletal system and organ development (FIGS. 6C and 6F). Day 28 and 42 chondrospheroids, which were the most similar, were enriched for genes involved in skeletal system development and morphogenesis; however, day 28 chondrospheroid expression showed an enrichment for collagen and extracellular matrix organization (FIG. 6D), evidenced by transiently high expression of COL2A1, COL3A1, COL9A1, COL11A1, and COL11A2 (FIGS. 6F and 15B). In contrast, day 42 chondrospheroids showed enrichment for genes involved in cartilage and connective tissue development (FIG. 6E), showing higher expression of COMP, GDF5, MGP, and PRG4 and sustained expression of ACAN, DCN, DPT. FMOD, and IGFBP7 (FIG. 6F). Notably, these transcriptional profiles matched protein expression (see FIGS. 5B and 5E) with early, transient COL1A1 expression; progressive expression of ACAN, and PRG4; uniformly low expression of COL10A1 (with the exception of one replicate); and absence of ALPL expression (FIGS. 6F and 15B). BMP activation in chondrospheroids led to the induction of ID genes with low expression of osteogenic genes (COL1A1, SPP1, IBSP, ALPL) similar to hBMSC/SSC-derived chondrocytes that comprise stable hyaline-like cartilage (see FIGS. 4A-D). Taken together, these results provide transcriptional confirmation of sclerotome and chondrogenic identity using a novel, serum-free chondrospheroid differentiation protocol with selective, timed BMP activation.

### Example 10

### Chondrospheroid Transcriptomes Reveal a Fetal-Like Chondrogenic Identity

To determine the chondrogenic maturity of cultured chondrospheroids, the datasets were normalized and compared with primary human embryonic (5-6 weeks-old), fetal (17 weeks-old), adolescent, and adult chondrocytes from a recent study (Ferguson et al., Nature Communications, 9(1), doi.org/10.1038/s41467-018-05573-y, 2018). Both PCA and hierarchical clustering confirmed the transcriptional similarity among adolescent and adult chondrocytes reported in the initial study (Ferguson et al., Nature Communications, 9(1), doi.org/10.1038/s41467-018-05573-y, 2018)(FIGS. 6G-H). PCA placed embryonic chondrocytes at the intersection of sclerotome and day 14 chondrospheroids with a deviation towards sclerotome cells; fetal chondrocytes were near day 28 and 42 chondrospheroids with day 42 chondrospheroids approaching adult chondrocyte populations (FIG. 6G). Hierarchical clustering grouped sclerotome cells with embryonic chondrocytes and all chondrospheroid datasets with fetal chondrocytes (FIG. 6H); consistently, Smear plot analysis confirmed the most transcriptional conservation among chondrospheroids and fetal chondrocytes (FIG. 15C). Therefore, the initial chondrogenic differentiation of sclerotome cells mimics an embryonic chondrogenic state, which progresses to a fetal-like state in day 28 and 42 chondrospheroids.

### Example 11

Chondral Transplantation of hiPSC/HyA-FMB Constructs Yields Stable Chondrogenesis

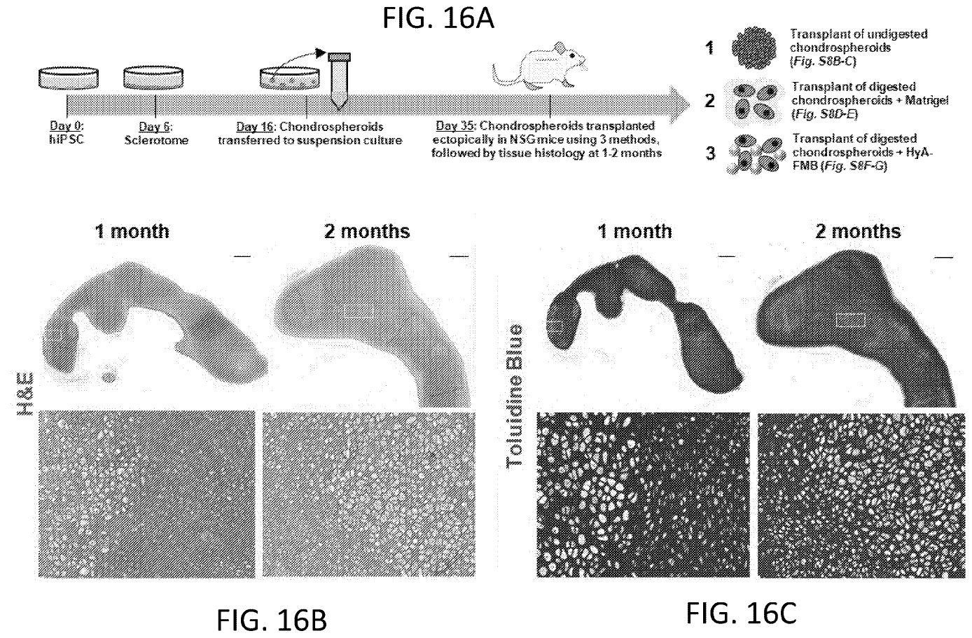

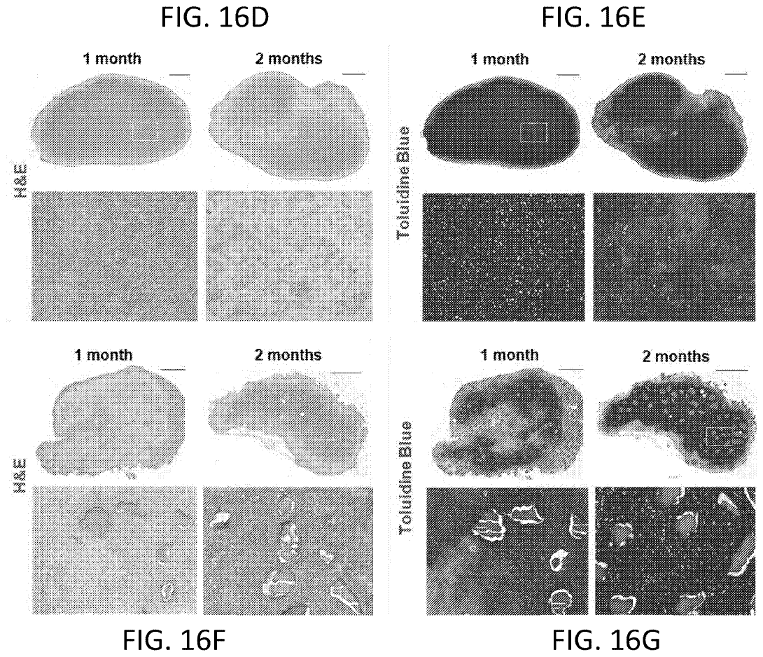

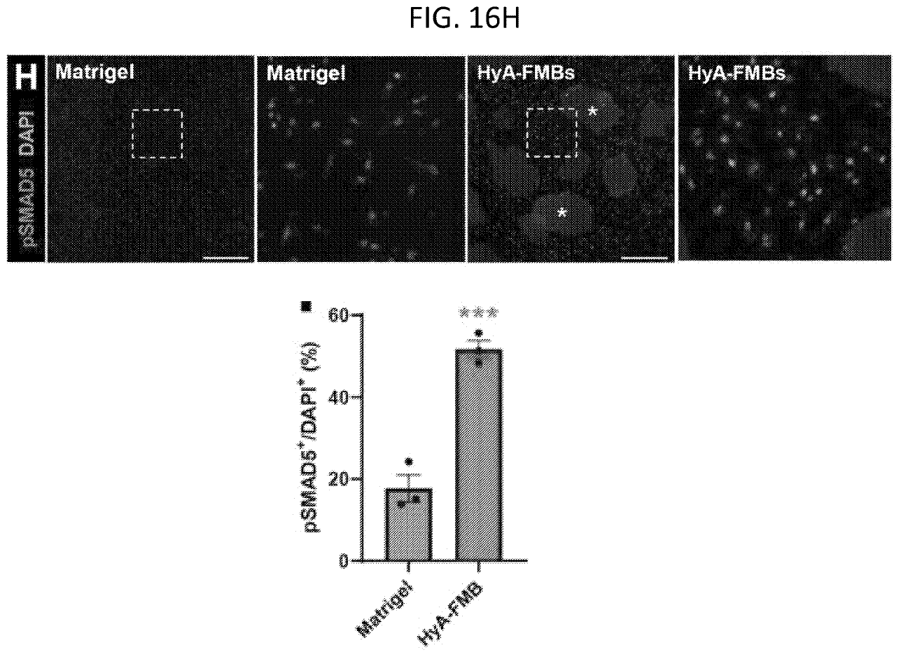

To examine stable chondrogenesis in transplanted chondrospheroids, subcutaneous transplantation was used to test three cell delivery methods (FIG. 16A). The first method involved the transplantation of undigested day 35 chondrospheroids, which led to patchy hypertrophy by 1 month and more widespread hypertrophy at 2 months (FIGS. 16B-16C). The latter two methods involved enzymatic digestion of day 35 chondrospheroids, and transplantation of isolated cells either in Matrigel or with HyA-FMB scaffolds. Chondrospheroid cells transplanted in Matrigel formed a uniform cartilage matrix by 1 month, which became patchy by 2 months with minimal evidence of hypertrophy (FIGS. 16D-16E). In contrast, HyA-FMB transplants formed a uniform metachromatic stain of cartilage matrix by Toluidine Blue at 2 months (FIGS. 16F-16G), suggesting improved chondrogenic stability in vivo. The improved chondrogenesis when cells derived from chondrospheroids were attached to HyA-FMBs as opposed to MATRIGEL™ is supported by the fact that pSMAD5 was present in the HyA-FBS transplants, but not in the MATRIGEL™ transplants as seen in FIG. 16H.

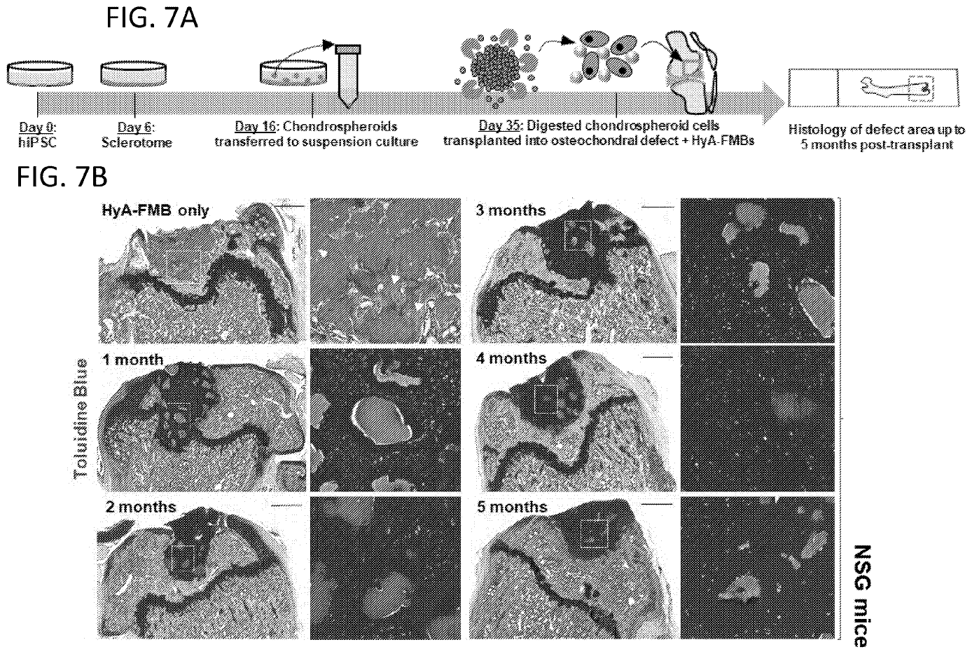

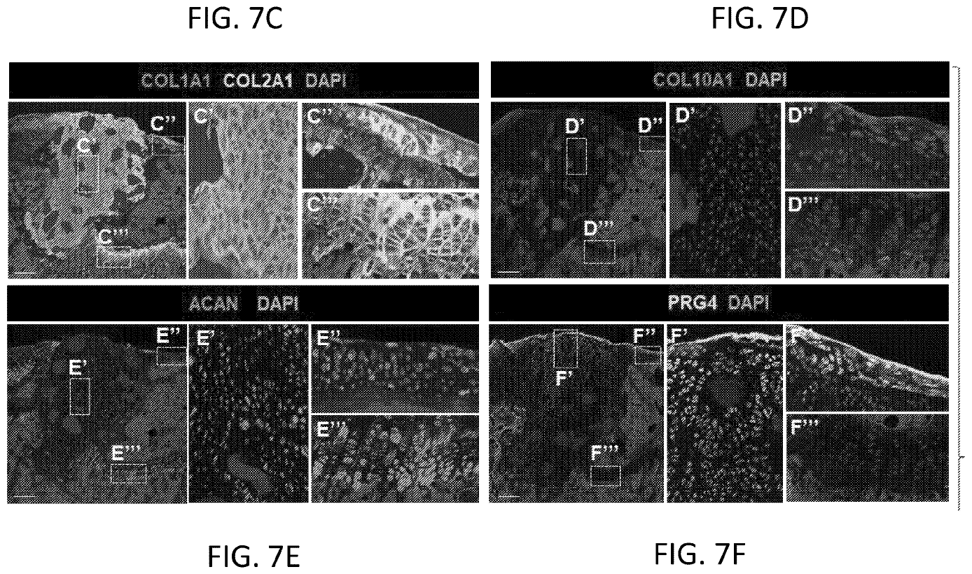

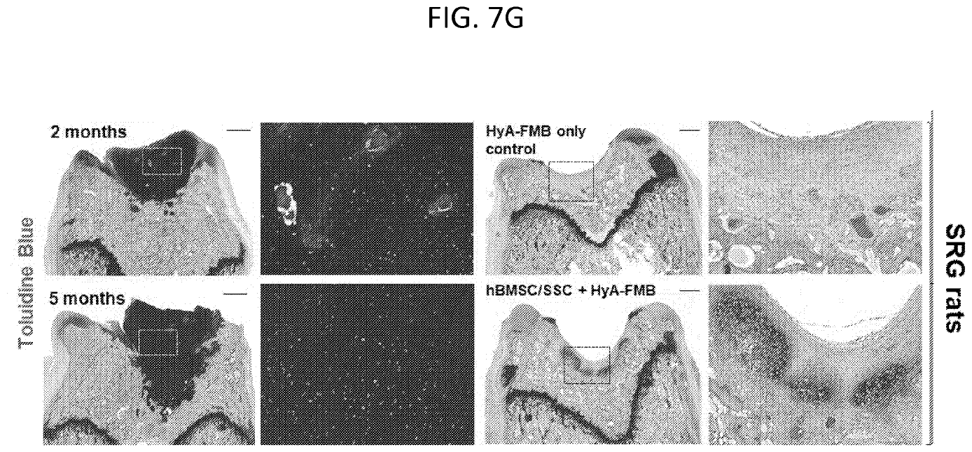

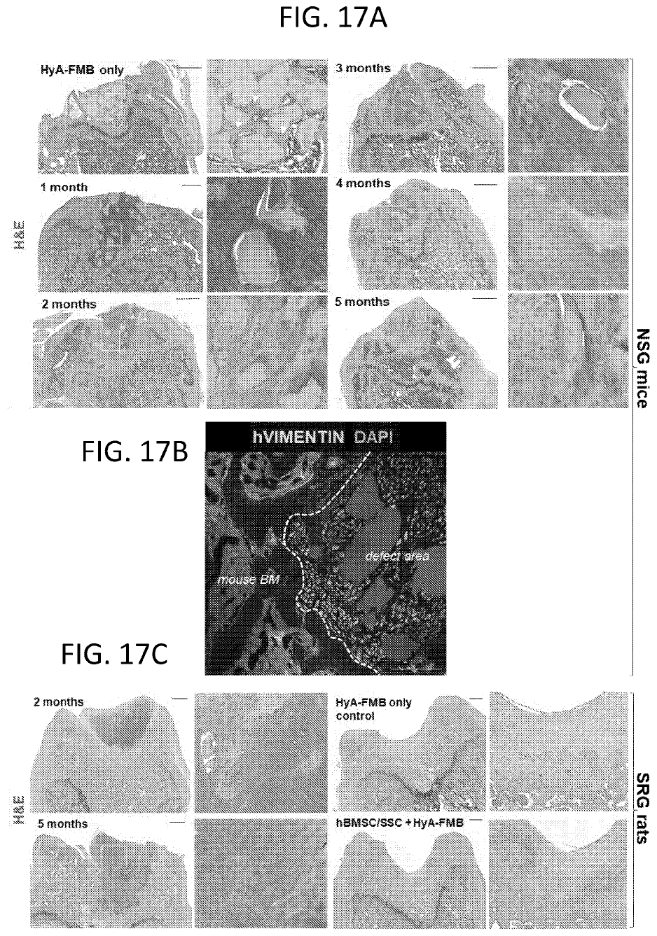

Digested chondrospheroid cells attached to HyA-FMBs were transplanted into articular cartilage defects in vivo (FIG. 7A). Day 35 chondrospheroid cells attached to HyA-FMBs formed stable, hyaline-like cartilage for up to 5 months upon chondral transplantation in NSG mice compared with controls which formed bone at the defect site (FIGS. 7B and 17A). These transplanted human cells, which were confirmed by hVIMENTIN expression (FIG. 17B), exhibited similar protein expression to surface articular chondrocytes and disparate expression to growth plate chondrocytes; i.e, immunofluorescence analyses showed uniform COL2A1 expression, no COL10A1 expression, widespread ACAN expression, and surface PRG4 expression in transplanted chondrospheroid cells, confirming an articular-like phenotype (FIGS. 7C-F). Similarly, chondral transplantation of day 35 chondrospheroid cells attached to HyA-FMBs in SRG rats produced dense metachromatic staining with Toluidine Blue after 5 months at the defect site, which proved superior to patchy, hypertrophic chondrogenesis and fibrocartilage formation seen with transplantation of hBMSC/SSC/HyA-FMB constructs and absent chondrogenesis in controls (FIGS. 7G and 17C). Overall, these data confirm stable chondrogenesis in two animal models with a defined, serum-free, articular-like cell source obtained from timely and selective BMP activation.

Thus, it was confirmed that hBMSCs/SSCs attached to HyA-FMBs generate stable, hyaline-like cartilage upon ectopic transplantation, and the HyA-FMB model was used to examine mechanisms that guide stable cartilage formation using scRNA-seq. HyA-FMBs promote early expression of collagenous and non-collagenous proteins, including COMP, DPT, IGFBP5, and MGP. BMP signaling is globally decreased in HyA-FMB organoids at days 1 and 3, but is restored and activated at days 5 and 10 in pre-chondrogenic cells that express high levels of IGFBP5 and MGP and low levels of hypertrophic and osteogenic markers. Consistently, chronic suppression of the BMP-ID axis led to a progressive increase in osteogenic transcription, while BMP activating genes were globally increased in stable, hyaline-like cartilage 8 weeks after ectopic transplantation. These results suggested that stable cartilage development is guided by initial BMP inhibition followed by BMP activation in pre-chondrogenic cells.

This mechanism was employed in a serum-free hiPSC differentiation protocol to generate stable articular chondrocyte-like cells. Following efficient differentiation to sclerotome cells enriched in IGFBP5, monolayer cultures were treated with the BMP activators, BMP-2 and GDF-5, led to the formation of loosely attached nodules with high SOX9 expression, termed chondrospheroids. BMP activation in purified chondrospheroids produced strong COL2A1, ACAN, and PRG4 expression with little to no COL10A1 expression by 42 days, yet BMP activation in all sclerotome cells led to the induction of hypertrophic and osteogenic markers, ALPL and COL10AL. Further, transplantation of chondrospheroid cells, which resembled a fetal articular chondrocyte-like phenotype in RNA sequencing studies, attached to HyA-FMBs into a chondral defect produced stable hyaline-like cartilage for up to 5 months in NSG mice and SRG rats. Overall, these studies signify the biphasic nature of BMP-ID signaling in the formation and maintenance of stable cartilage and provide an articular-like cell source for clinical and regenerative OA therapies.

hBMSCs/SSCs have the capacity to form bone, hematopoietic-supporting stroma, and adipocytes following ectopic transplantation, yet their chondrogenic capacity is fully appreciated in pellet cultures supplemented with TGFβ (Robey & Riminucci, 2020). TGFβs are expressed at the developing joint interzone; are known to stimulate proliferation and inhibit osteogenesis and adipogenesis in hBMSCs/SSCs; and promote chondrogenic cell fate in hBMSCs/SSCs after one day of exposure (Alliston et al., Nature Communications, 9(1). doi.org/10.1038/s41467-018-05573-y, 2001; Futrega et al., Communications Biology, 4(1), 1-12, doi.org/10.1038/s42003-020-01520-0, 2021; Kumar et al., Endocrinology, 153(1), 254-263, doi.org/10.1210/en.2011-1169, 2012; Spagnoli et al., Journal of Cell Biology, 177(6), 1105-1117, doi.org/10.1083/jcb.200611031, 2007). Consistently, it was shown that both control and HyA-FMB organoids require TGFβ supplementation for development of the glycan-rich cartilage matrix. However, TGFβ alone was insufficient in promoting a robust response in chondrogenic differentiation of hBMSCs/SSCs and hiPSCs, consistent with previous reports (De Kinderen et al., supra, 2022). About 90% of hBMSC/SSC-derived cells exposed to TGFβ1 expressed the osteogenic marker, IBSP, by day 10 of chondrogenic differentiation. Further, treatment of hiPSC-derived sclerotome cells with TGFβ1 alone was inefficient in chondrogenic differentiation, evidenced by stable COL1A1 and patchy ACAN expression. Examination of the TGFβ signaling pathway in early HyA-FMB organoids revealed increased expression of TGFs inhibitors (RBX1 and SKP1) and BMP inhibitors (GREM1 and NBL1), the latter of which remained elevated by day 3 of chondrogenic differentiation. At days 5 and 10, HyA-FMBs restored and activated BMP signaling in a pre-chondrogenic group of cells, which were enriched for IGFBP5 and MGP and expressed low levels of osteogenic and hypertrophic markers. These mechanistic studies are consistent with IGFBP5's role in preventing osteogenic transcription and mineralization during BMP-2-induced osteogenic differentiation (Mukherjee & Rotwein, Molecular Endocrinology, 22(5), 1238-1250, doi.org/10.1210/me.2008-0001, 2008).

Transplantation of undigested chondrospheroids led to progressive hypertrophy by 2 months, but adding HyA-FMBs to digested chondrospheroid cells prior to transplantation produced stable articular chondrocyte-like cells that express ACAN, COL2A1 and PRG4 and stable cartilage matrix for up to 5 months. Similarly, transplantation of pre-formed HyA-FMB organoids led to the formation of fibrocartilage and bone, but transplantation of hBMSCs/SSCs attached to HyA-FMBs without chondrogenic induction produced stable, hyaline-like cartilage when HyA-FMBs remained at the transplant site. These results emphasize the role of the HyA-FMBs in stable chondrogenesis in vivo and demonstrate superior cartilage formation when single cells are transplanted with HyA-FMBs rather than whole organoids and tissues. It is possible that hyaluronic acid present on scaffold is aiding in stable chondrocyte maturation of fetal-like cells similar to the events observed during joint cavitation (Archer et al., Birth Defects Research Part C—Embryo Today: Reviews, 69(2), 144-155, doi.org/10.1002/bdrc.10015, 2003; Waddell et al., Clinical Orthopaedics and Related Research, 465(465), 241-248, doi.org/10.1097/BLO.0b013e31815873f9, 2007). Further, larger tissues often contain diffusion gradients, leading to differences in signaling, extracellular matrix composition, and cell phenotype across the tissue (Futrega et al., Biomaterials, 62, 1-12. https://doi.org/10.1016/j.biomaterials.2015.05.013, 2015; Toda et al., Science, 361(6398), 156-162, doi.org/10.1126/science.aat0271, 2018). For this reason, digested cells or smaller microtissues with more homogeneity, similar to the microwell-mesh system previously described (Futrega et al., Communications Biology, 4(1), 1-12, doi.org/10.1038/s42003-020-01520-0, 2015), may provide superior clinical efficacy and reproducibility. Finally, transplantation of hiPSC-derived chondrospheroid cells proved superior to hBMSC/hSSC-derived cells, which may suggest the priming of hBMSCs/SSCs toward a differentiation pathway leading to endochondral ossification not present in hiPSC-derived cells (Somoza et al., Tissue Engineering—Part B: Reviews, 20(6), 596-608, doi.org/10.1089/ten.teb.2013.0771, 2014). The data show that TGFβ, BMP-2, and GDF-5 promote efficient chondrogenesis in SOX9+ chondrospheroids, which promote stable cartilage at the joint surface up to 5 months upon attachment to HyA-FMB scaffolds, providing a regenerative therapy for discases of the joints, such as OA.

### Example 12

### Materials and Methods

Chondrogenic Differentiation of hBMSCs/SSC-derived Organoids: To generate control organoids, 2.5×10⁵ hBMSCs/SSCs (passage 2) were suspended in 1 mL chondrogenic medium—which consisted of high glucose Dulbecco's Modified Eagle Medium (DMEM) with sodium pyruvate (ThermoFisher, Cat. No. 11995073), 1% insulin-transferrin-selenium (ThermoFisher, Cat. No. 51500056), 100 nM dexamethasone, 50 ug/mL L-ascorbic acid phosphate magnesium salt n-hydrate, 1% penicillin/streptomycin, and 10 ng/mL TGFβ1 (Peprotech, Cat. No. 100-21)—in 14 mL conical tubes. The same process was employed to generate HyA-FMB organoids, except each conical tube contained 3 mg of pre-sterilized HyA-FMBs (70% ethanol washes overnight), which were constructed according to established protocols (Kuznetsov et al., Stem Cells Translational Medicine, 26, 586-592. //doi.org/10.1016/j.joca.2018.02.293, 2019). For both control and HyA-FMB organoids, each tube was centrifuged at 193×g for 6 minutes, then incubated with loosened caps at 37° C., 5% CO₂ with medium replacements 2-3×/week.

### Tissue Processing and Toluidine Blue/H&E Staining:

Tissue Processing. hBMSC/SSC and hiPSC-derived tissues from in vitro studies were fixed in 4% formaldehyde at room temperature with mixing for 2 hours, then embedded in 3% agarose disks and placed in 70% ethanol for paraffin embedding. Mouse subcutaneous and chondral transplants were fixed overnight in 4% formaldehyde at 4° C. with mixing, and demineralized with 250 mM EDTA/dH2O solution (Quality Biological, Cat. No. 351-027-101) for 1.5-2 weeks at 4° C. with mixing. Rat chondral transplants were fixed for 2 days and demineralized for 4 weeks in the same conditions. Transplants were stored in 70% ethanol at 4° C. and embedded in paraffin within one week.

Toluidine Blue Staining. Tissues were sectioned at 7 μm and rehydrated in two washes of xylene for 5 minutes, 100% ethanol for 2 minutes, 95% ethanol for 2 minutes, and one wash in tap water for 2-3 minutes. Then, tissues were stained with 0.04% toluidine blue in acetate buffer for 4 minutes (Sigma, Cat. No. 89640), rinsed with water twice for 1 minute, and placed in xylene for mounting with Optic Mount I (Mercedes Scientific, Cat. No. MER7722).

H&E Staining. Rehydrated tissues were treated with the following reagents (all from Fischer Scientific): Hematoxylin 2 (Cat. No. 22050113)(1×3 minutes), tap water (2×2 minutes), Clarifier 2 (Cat. No. 22050117)1×1 minute), tap water (2×1 minute), Bluing reagent (Cat. No. 22050114)(1×1 minute), tap water (1×1 minute), 95% ethanol (1×1 minute), Eosin Y with Phloxine (Cat. No. 22050198)(1×40 seconds), 10 brief washes in 95% ethanol, 100% ethanol (1×1 minute), xylene (2×3 minutes), and mounting with Optic Mount I.

Single-cel RNA Sequencing and Analysis: Passage 2 hBMSCs/SSCs from donors 1-2 (see Table 1) were used to generate organoids for single cell RNA sequencing studies, and were shown to form bone, hematopoietic supporting-stroma, and adipocytes upon ectopic transplantation.

TABLE 1
hBMSC/SSC donors

| Donor | Age | Sex | Part of | Diagnosis | Experiment |
|---|---|---|---|---|---|
| 1 | 14 | Female | Right hip | Right hip | scRNA-seq, |
| | | | | dislocation/spastic | |
| 2 | 18 | Male | Femur | Myopathy | scRNA-seq |
| 3 | 8 | Male | Femur | Cerebral palsy | scRNA-seq |
| 4 | 4 | Female | Femur | Sickle cell anemia | Transplant, |
| 5 | 13 | Male | Right | Macrophage | ID inhibition |
| | | | maxilla | activation syndrome | |
| 6 | 52 | Male | Right iliac | Fibrous dysplasia | ID inhibition |

Control and HyA-FMB organoids from days 1-10 and ectopic HyA-FMB transplant were digested in high-glucose DMEM containing 0.2% collagenase II (ThermoFisher, Cat. No. 17101015), 0.1% hyaluronidase (Merck, Cat. No. H3506), and 0.1% dispase (Sigma, Cat. No. D4693) with gentle pipetting every 20-30 minutes. Enzymatic digestion was observed in 6 well-plates, and digestion was terminated when aggregates were completely dissolved, for up to 2 hours. Enzyme was inhibited with 20% FBS, and cells were filtered through 70 μm strainers and re-suspended in PBS supplemented with 0.04% high-quality bovine serum albumin (BSA, Miltenyi Biotec, Cat. No. 130-091-376) at 1000 cells/μL for single-cell capture. Only samples with >95% viability were submitted for single-cell capture, as measured by Vi-CELL BLU Cell Viability Analyzer (Beckman Coulter). Naïve hBMSC/SSC controls (from donor 3) required a 10-minute incubation with collagenase II on monolayer cultures prior to cell detachment and cell capture. Single-cell RNA sequencing was performed using 10× Genomics Chromium instruments. Reads were mapped using GRCh38-2020-A reference transcriptome, and libraries were processed using Cell Ranger version 7.0. The following quality control measurements were performed: Ambient RNA removal using SoupX (Young & Behjati, 2020), cell cycle regression using Seurat's Cell Cycle Scoring (satijalab.org/seurat/articles/cell_cycle_vignette), doublet identification and removal using scDblFinder (Germain, Robinson, Lun, Garcia Meixide, & Macnair, 2022), and removal of low-quality or dead cells that expressed fewer than 150-200 genes and >10-15% mitochondrial reads. Captures from the HyA-FMB ectopic transplant contained an abnormally high number of doublets, so only global transcriptional measurements were described from this dataset. The merging of datasets using anchoring techniques (satijalab.org/seurat/articles/integration_introduction), dimensionality reduction, cluster analysis and UMAP projection was performed using Seurat's R package with cluster resolution determined by Clustree (Zappia & Oshlack, GigaScience, 7(7), 1-9, doi.org/10.1093/gigascience/giy083, 2018). Pseudotime analyses of integrated datasets were performed using Monocle3 (Cao et al., Nature, 566(7745), 496-502, doi.org/10.1038/s41586-019-0969-x, 2019) with naïve hBMSC/SSC datasets chosen as the root/origin of each trajectory. Pathway analysis was performed using clusterProfiler and pathview, with focused analysis on KEGG signal transduction pathways (Wu et al., The Innovation, 2(3), 100141, doi.org/10.1016/j.xinn.2021.100141, 2021). Analyses from days 1-3 were presented as whole dataset comparisons, as the emergence of chondrogenic/osteogenic populations was not clearly delineated until day 5. Differentially expressed genes, which represented at least 25% of each cluster (p<0.05), were analyzed using gProfiler (biit.cs.ut.ee/gprofiler/gost) for functional enrichment analysis and cell cluster annotation.

### Immunofluorescence and Confocal Imaging

Staining Procedure. Paraffin-embedded tissues were used for immunofluorescence. Tissues were sectioned at 7 μm and heated for 1 hour at 60° C., then rehydrated in 2 washes of xylene (5 minutes), 100% ethanol (5 minutes), 95% ethanol (5 minutes), 70% ethanol (5 minutes), and distilled water (5 minute). Two different antigen retrieval methods were used: (1) for staining with COL1A1, COL2A1, COL10A1, PRG4, and hVIMENTIN, tissues were treated with 2 mg/mL hyaluronidase/PBS solution for 1 hour at 37° C., followed by a PBS wash and treatment with Pepsin (Millipore Sigma, Cat. No. R2283) for 5-10 minutes at 37° C.; (2) for staining with MGP, COMP, DPT, ACAN, and ALPL, tissues were incubated in citrate buffer (Millipore Sigma, Cat. No. C9999) for 1 hour at 70° C., followed by a 15-second wash with distilled water. Cytofix/Cytoperm buffer (BD, Cat. No. 554722) was used in some cases to enhance intracellular staining. Tissues were blocked in staining solution—PBS supplemented with 5% donkey serum (Sigma, Cat. No. D9663) and 0.5% IgePal (Sigma, Cat. No. I3021)—for 30 minutes, then stained with primary antibodies overnight at 4° C. On the following day, tissues were washed in PBS (4× for 5 minutes) and incubated with secondary antibodies for 1 hour at room temperature, followed by a PBS wash and mounting with Prolong Gold Antifade Mountant with DAPI (ThermoFisher, Cat. No. P36931). For negative controls, non-immune antibodies of the same isotype and host species as primary antibodies were incubated overnight at 4° C., followed by secondary staining. The following primary antibodies were used: Rabbit anti-MGP (Abcam, Cat. No. ab224367), Rabbit anti-COMP (Abcam, Cat. No. ab231977), Rabbit anti-DPT (ThermoFisher, Cat. No. PA514396), Rabbit anti-COL1A1 (Abcam, Cat. No. ab34710), Mouse anti-COL2A1 (Developmental Studies Hybridoma Bank, Cat. No. II-116B3), Rabbit anti-COL10A1 (synthesized in the Skeletal Biology Section in consultation with Dr. Larry W. Fisher, according to Kuznetsov et al., supra, 2019), Rabbit anti-ACAN (Millipore Sigma, Cat. No. AB1031), Goat anti-ALPL (ThermoFisher, Cat. No. PA547419), Rabbit anti-PRG4 (Abcam, Cat. No. ab28484), and Alexa 488 Goat anti-hVIMENTIN (R&D Systems, Cat. No. IC8104G). The following secondary antibodies were used: Alexa 546 Donkey anti-Rabbit (ThermoFisher, Cat. No. A10040), Alexa 488 Donkey anti-Mouse (ThermoFisher, Cat. No. R37114), and Alexa 488 Donkey anti-Goat (Abcam, Cat. No. ab150129).

Confocal Imaging. Tiled z-stacks were acquired using the Zeiss LSM 880 confocal microscope with Airyscan using the 20× objective and 20% overlap. Maximum intensity projections were merged in Fiji/Image J (National Institutes of Health) for image analysis and quantifications. Second harmonic generation images were acquired using a Nikon A1R+MP, two photon, resonant scanner with the 40× lens water objective. Live-cell imaging of SOX9-mCherry hiPSCs was performed using Essen Incucyte Zoom S3 with 2018B software.

### ID Inhibition and Knockdown Studies

Pan-ID inhibition. Passage 2 hBMSCs/SSCs from donors 5-6 were used to generate organoids for pan-ID inhibition studies (see Table 1). 2.5×10⁵ hBMSCs/SSCs were suspended in 1 mL chondrogenic medium (see above) treated with 30 μM AGX51. Treatment with AGX51 and ethanol vehicle was continued every 2-3 days for 28 days, and triplicates for each group were processed for qRT-PCR at days 3, 7, 14, 21, and 28.

ID1 Knockdown. Passage 2 hBMSCs/SSCs from donor 4 was used for knockdown studies (see Table 1). Short hairpin RNA (shRNA) against ID1 (siID1) and control short hairpin RNA (siGL), which were cloned into a lentiviral construct containing GFP according to previous reports (Wagner et al., Proceedings of the National Academy of Sciences of the United States of America, 103(16), 6338-6343, https://doi.org/10.1073/pnas.0508143103, 2006), and packaging constructs were obtained from the National Cancer Institute, Frederick, MD. Passage 14 HEK 293T cells were grown to ~70% confluency on 150 mm dishes and transfected with 50 μg transfer plasmid (siID1 or siGL), 30 μg packaging plasmid (HIV), 10 μg envelope plasmid (VSVG), and 420 μg polyethylenimine (PEI) in 10 mL Advanced DMEM (ThermoFisher, Cat. No. 12491-015) for a brief 5-minute incubation without serum, then diluted to 30 mL with Advanced DMEM supplemented with 10% FBS for 18 hours. The culture medium was replaced the following day, collected after 48 and 72 hours, and filtered using 0.45 μm PES filters (Merck, Cat. No. SLHAR3355). For transduction, hBMSCs/SSCs were incubated in αMEM supplemented with 20% FBS, 20 μg/mL protamine sulfate (APP Pharmaceuticals), and viral supernatant (1:5) for 72 hours. GFP⁺ transduced hBMSCs/SSCs were FACS-sorted, as noted below, and expanded for generation of organoids and chondrogenic differentiation up to 28 days.

Quantitative Real-Time PCR (qRT-PCR) and RNA Sequencing

qRT-PCR. Cartilage organoids were homogenized mechanically using plastic plungers in RNAse free microfuge tubes. Lysates were processed according to Qiagen's RNeasy Mini Kit with DNAse treatment (Cat. No. 74004), and RNA quality and concentration was measured using NanoDrop. cDNA was constructed using SuperScript III First-Strand Synthesis SuperMix (ThermoFisher, Cat. No. 11752050) with no Reverse Transcriptase added to negative controls. Samples were measured in triplicate using the Quantstudio 6 Flex system (Applied Biosystems), and ΔΔCT values were calculated using GAPDH as internal controls. Primer sets used for qRT-PCR are listed in Table 2.

| | SEQ ID NO | | SEQ ID NO | |
|---|---|---|---|---|
| | (Forward | | (Reverse | |
| Gene | Primer) | Forward (5′-3′) | Primer) | Reverse (5′-3′) |
| RUNX | SEQ ID NO: 1 | GGAGTGGACGAGGCAAG | SEQ ID NO: 2 | AGCTTCTGTCTGTGCCTTCTG |
| IBSP | SEQ ID NO: 3 | AACGATTTCCAGTTCAGG | SEQ ID NO: 4 | AGCCCAGTGTTGTAGCAGAA |
| COL1 | SEQ ID NO: 5 | CAGCCGCTTCACCTACAG | SEQ ID NO: 6 | TTTTGTATTCAATCACTGTCT |
| COL1 | SEQ ID NO: 7 | ACTCCCAGCACGCAGAA | SEQ ID NO: 8 | TGGGCCTTTTATGCCTGTGGG |
| SOX9 | SEQ ID NO: 9 | ACTCCTCCTCCGGCATGA | SEQ ID NO: 10 | GCTGCACGTCGGTTTTGG |
| ACAN | SEQ ID NO: 11 | TCGAGGACAGCGAGGCC | SEQ ID NO: 12 | TCGAGGGTGTAGCGTGTAGA |
| COL2 | SEQ ID NO: 13 | GGCAATAGCAGGTTCAC | SEQ ID NO: 14 | CGATAACAGTCTTGCCCCACT |
| OCT4 | SEQ ID NO: 15 | AGTGAGAGGCAACCTGG | SEQ ID NO: 16 | ACACTCGGACCACATCCTTC |
| MIXL1 | SEQ ID NO: 17 | GGTACCCCGACATCCACT | SEQ ID NO: 18 | TAATCTCCGGCCTAGCCAAA |
| BRAC | SEQ ID NO: 19 | TGCTTCCCTGAGACCCAG | SEQ ID NO: 20 | GATCACTTCTTTCCTTTGCAT |
| GSC | SEQ ID NO: 21 | GAGGAGAAAGTGGAGGT | SEQ ID NO: 22 | CTCTGATGAGGACCGCTTCTG |
| EOME | SEQ ID NO: 23 | CAACATAAACGGACTCA | SEQ ID NO: 24 | ACCACCTCTACGAACACATT |
| FOXA | SEQ ID NO: 25 | GGGAGCGGTGAAGATGG | SEQ ID NO: 26 | TCATGTTGCTCACGGAGGAG |
| MESP | SEQ ID NO: 27 | GAAGTGGTTCCTTGGCAG | SEQ ID NO: 28 | TCCTGCTTGCCTCAAAGTGT |
| MESP | SEQ ID NO: 29 | AGCTTGGGTGCCTCCTTA | SEQ ID NO: 30 | TGCTTCCCTGAAAGACATCA |
| FOXF | SEQ ID NO: 31 | AGCAGCCGTATCTGCACC | SEQ ID NO: 32 | CTCCTTTCGGTCACACATGCT |
| TBX6 | SEQ ID NO: 33 | AAGTACCAACCCCGCAT | SEQ ID NO: 34 | TAGGCTGTCACGGAGATGAA |
| CDX2 | SEQ ID NO: 35 | GGGCTCTCTGAGAGGCA | SEQ ID NO: 36 | CCTTTGCTCTGCGGTTCTG |
| MSGN | SEQ ID NO: 37 | CGGAATTACCTGCCACCT | SEQ ID NO: 38 | GGTCTGTGAGTTCCCCGATG |
| DLL1 | SEQ ID NO: 39 | ACTCCGCGTTCAGCAACC | SEQ ID NO: 40 | TGGGTTTTCTGTTGCGAGGTC |
| FOXC | SEQ ID NO: 41 | CCTCCTGGTATCTCAACC | SEQ ID NO: 42 | GAGGGTCGAGTTCTCAATCC |
| PARA | SEQ ID NO: 43 | GAGCTGAGGAGAGTCCC | SEQ ID NO: 44 | TGTGCCTCTCTCTAGGTCCA |
| MEOX | SEQ ID NO: 45 | TCTGAGCGCCAGGTCAA | SEQ ID NO: 46 | CTGAACTTGGAGAGGCTGTG |
| BAPX1 | SEQ ID NO: 47 | GATTTCAGGCCTGCTGGG | SEQ ID NO: 48 | TTTCGCACCCCTTGGTTACA |
| NKX3. | SEQ ID NO: 49 | CCAGCTCAGGTGACAAC | SEQ ID NO: 50 | CTTGGCCCCTTGTGCTTTTC |
| PAX3 | SEQ ID NO: 51 | CTCCACGCTCCGGATAGT | SEQ ID NO: 52 | ATCTTGTGGCGGATGTGGTT |
| PAX9 | SEQ ID NO: 53 | TGGTTATGTTGCTGGACA | SEQ ID NO: 54 | GGAAGCCGTGACAGAATGAC |
| HAND | SEQ ID NO: 55 | GTGCGTCCTTTAATCCTC | SEQ ID NO: 56 | GTGAGAGCAAGCGGAAAAG |
| ISL1 | SEQ ID NO: 57 | AGATTATATCAGGTTGTA | SEQ ID NO: 58 | ACACAGCGGAAACACTCGAT |
| TBX5 | SEQ ID NO: 59 | TACCACCACACCCATCAA | SEQ ID NO: 60 | ACACCAAGACAGGGACAGAC |
| PRRX1 | SEQ ID NO: 61 | TGATGCTTTTGTGCGAGA | SEQ ID NO: 62 | AGGGAAGCGTTTTTATTGGCT |
| HOXB | SEQ ID NO: 63 | AACTCCTTCTCGGGGCGT | SEQ ID NO: 64 | CATCCCATTGTAATTGTAGCC |
| TBX20 | SEQ ID NO: 65 | GGCGACGGAGAACACAA | SEQ ID NO: 66 | CTGGGCACAGGACGACTTC |
| NKX2. | SEQ ID NO: 67 | CAAGTGTGCGTCTGCCTT | SEQ ID NO: 68 | CAGCTCTTTCTTTTCGGCTCT |
| GAPD | SEQ ID NO: 69 | ATGGGGAAGGTGAAGGT | SEQ ID NO: 70 | TAAAAGCAGCCCTGGTGACC |

RNA Sequencing. RNA from hiPSCs, sclerotome cells, and chondrospheroids (days 14, 28, and 42) was isolated using Qiagen's RNeasy Mini Kit with DNAse treatment. mRNA libraries were prepared using the polyA TruSeq method (Illumina) and sequenced on an Illumina NextSeq500 configured for 100 paired-end reads. FASTQ files were pre-processed using the snakemake/5.6.0 utility and aligned using the STAR v2.7.3a aligner with mapping parameters derived from the GENCODE project. All samples had >50% alignment with <1% reads mapping to ribosomal transcripts. Sequencing depth at 50 million reads was shown to be sufficient in saturation curves. Quantification, normalization, PCA, hierarchical clustering, and differential expression analysis were performed using Bioconductor's edgeR package (Robinson, McCarthy, & Smyth, 2009). Top 100 differentially expressed genes from each dataset were assessed against top 5 gene ontology pathways in gProfiler (Raudvere et al., Nucleic Acids Research, 47(W1), W191-W198. doi.org/10.1093/nar/gkz369, 2019), which reduced the list to 103 relevant genes for plotting using the heatmap.2 function (cran.r-project.org/web/packages/gplots/index.html). The ComBat-Seq method (Yuging Zhang, Parmigiani, & Johnson, 2020) was employed to generate batch-corrected expression matrices for normalization of previously published datasets (Ferguson et al., supra, 2018) with chondrospheroid datasets.

### Ectopic and Chondral Transplantation

Ectopic Transplantation. Subcutaneous transplantation in male NSG rats were performed according to an established method (Kuznetsov et al., supra, 2019; Robey & Riminucci, Skeletal stem cells: tissue-specific stem/progenitor cells of cartilage, bone, stroma, and marrow adipocytes, In Principles of Bone Biology (4th ed., pp. 45-71). Academic Press., 2020) under an NIDCR ACUC-approved protocol. Briefly, the dorsal skin of the back was shaved and sterilized using alternating Betadyne and ethanol scrubs. Then, a 1.5 cm incision was made on the back, and hBMSC/SSC- and hiPSC-derived cells were placed in a subcutaneous pocket using a sterile spatula, with up to 4 transplants per mouse. Three methods of cell delivery were used for day 35 chondrospheroids. The first method involved the placement of undigested chondrospheroids directly into the subcutaneous pocket. The latter two methods involved the digestion of chondrospheroids using 0.2% collagenase II and 0.1% dispase in high glucose DMEM for up to 2 hours with gentle pipetting every 30 minutes, after which cells were washed and filtered through 70 μm strainers. The second approach involved the re-suspension of ~1.7×10′ cells in 100 μL Matrigel (Corning, Cat. No. 356237). The final approach involved incubation of ~1.7×10⁶ cells with 10 mg pre-sterilized HyA-FMBs for 1.5 hours at 37° C. with gentle rocking. In both cases, transplants were placed on ice for ~1-2 hours during transfer and preparation of surgical site, then placed into a subcutaneous pocket. Incisions were closed using resorbable sutures (Ethicon, 5-0 Monocryl, Cat. No. Y303H) in a horizontal mattress pattern and dissected 1-2 months later.

Chondral Transplantation. Passage 2 hBMSCs/SSCs from donors 1 and 4 (see Table 1) were detached using Trypsin/EDTA, and 3×10⁶ hBMSCs/SSCs were incubated with 30 mg pre-sterilized HyA-FMBs, according to previous studies (Kuznetsov et al., Stem Cells Translational Medicine, 26, 586-592, doi.org/10.1016/j.joca.2018.02.29, 2019). In some cases, hBMSCs/SSCs were subjected to chondrogenic differentiation for 10 days, as described above, prior to transplantation of pre-formed organoids. For hiPSC transplants, ~1.7×10⁶ cells were incubated with 10 mg HyA-FMBs, as noted above. Both immunocompromised NSG mice and SRG rats were used for chondral transplantation under a NIDCR ACUC-approved animal protocol, and both left and right knees were used, often with one knee serving as a HyA-FMB only control (no cells) or sham control (no cells or HyA-FMBs). Surgical draping (Vitality Medical, Cat. No. 005714) and autoclaved instruments, as well as alternating Betadyne and alcohol scrubs of the shaved surgical site, were employed to maintain sterility. An incision was made at the medial aspect of the knee through the overlying skin and joint capsule with the mouse in a supine position. Next, the patella was gently dislocated laterally using a 25G needle, exposing the trochlea of the femur, which is then stabilized with large, serrated tweezers clamped to a ring-stand. The Ideal micro-drill (CellPoint Scientific) was used to create a chondral defect using a 0.9 mm drill bit for mice and 2.1 mm drill bit for rats (FineScienceTools, Cat. No. 19007) on the trochlear surface, and a 0.5 mm micro-curette was used to introduce and pressurize hBMSCs/SSCs/HyA-FMB or hiPSC/HyA-FMB constructs into the defect. Finally, the patella was gently relocated; two vertical sutures (Ethicon, 5-0 Vicryl, Cat. No. J303H) were tied at the joint capsule and 2-3 sutures (Ethicon, 5-0 Monocryl, Cat. No. Y303H) were placed in a horizontal mattress pattern in the skin. Heat pads were placed underneath the surgical area and surgical recovery area to maintain core temperature, and animals were administered Buprenorphine (ZooPharm LLC) subcutaneously before surgery and as needed afterwards. Femurs were gently dissected at several timepoints and processed, as noted above.

Generation of MIXL1-GFP and SOX9-mCherry hiPSCs

MIXL1-GFP. The donor plasmid was synthesized and cloned into Puc57-Kan plasmid by Genscript Biotech Corp. (Piscataway, NJ). The donor contains the MIXL1 left homology arm, exon 2 with TGA stop codon removed and fused to neonGreen, rBGpA, CMVPuro, BGHpA and right homology arm. The donor has 5 bases changed to the 3′UTR to prevent cutting by CRISPR/Cas9 guides as follows with changes underlined: AGTGGATTCGGGAGAATTCGAGATAAGCTCTGAGAAGCCATGACTGACAGCCTGAGAGA (SEQ ID NO: 71). LentiCRISPR v2 (Addgene plasmid #52961) was used as the backbone to create the CRISPR-Cas9 plasmids. The following primers were used to clone the guides targeting the 3′UTR into the lentiCRISPR v2 according to Sanjana et. al., (Nature Methods, 11(8), 783-784. doi.org/10.1038/nmeth.3047, 2014): 5′-caccgTGAGGATTCTGGGAGAATTC-3′ (SEQ ID NO: 72, 1a-MIXL-CRISPR-3UTR) with 5′-aaacGAATTCTCCCAGAATCCTCAc-3′ (SEQ ID NO: 73, 1b-MIXL-CRISPR-3UTR); 5′-caccgAGAATTCGGGATAAGCTCTG-3′ (SEQ ID NO: 74, 2a-MIXL-CRISPR-3UTR) with 5′-aaacCAGAGCTTATCCCGAATTCTc-3′ (SEQ ID NO: 75, 2b-MIXL-CRISPR-3UTR).

SOX9-mCherry. The SOX9-mCherry donor plasmid was inserted into Puc57 plasmid by GenScript Biotech Corp. (Piscataway, NJ). The TGA stop codon in exon 3 of the SOX9 coding region was replaced with GGC, followed by mCherry in frame. The synthesized product was inserted into Xba1 to HindIII sites of Puc57. The CMV-puro-BGHpA, flanked by loxP sites (also synthesized by GenScript), was cloned into the Pac1 to Spe1 sites of the SOX9-mCherry donor to confer selection capability and excision if necessary. The donor was modified to prevent cutting by CRISPR/Cas9 guides as follows with changes underlined: TATACGAAGTTATACTAGTGGAGGCCTCCCACGA (SEQ ID NO: 76). LentiCRISPR v2 was a gift from Feng Zhang (Addgene plasmid #52961). The following primers were used to clone the guides into the LentiCRISPRv2 according to Sanjana et. al (2014): 5′-caccgCAGCTCACTCGACCTTGAGG-3′ (SEQ ID NO: 77, FWD) with 5′-aaacCCTCAAGGTCGAGTGAGCTGc-3′ (SEQ ID NO: 78, REV)[CRISPR Set 1], and 5′-caccgCTTGAGGAGGCCTCCCACGA-3′ (SEQ ID NO: 79, FWD) with 5′-aaacTCGTGGGAGGCCTCCTCAAGc-3′ (SEQ ID NO: 80, REV)[CRISPR Set 2].

Transfection and Screening. NCRM NL5 hiPSCs were grown in Nutristem (Stemgent) for 1-2 weeks prior to transfection with AMAXA mouse ES transfection kit (A-023), 5 μg donor and 5 μg CRISPR/Cas9 (2.5 μg each). Five to six days after transfection, the colonies were treated with 0.25 to 0.5 μg/ml puromycin (Thermofisher Scientific) for 2 to 3 days. Colonies were expanded and retreated with 0.25 to 0.5 μg/ml puromycin for 3 days for additional selection. Individual colonies were picked and expanded in E8 medium. During passaging, some cells from each clone were used to test for the correct insertion of the donor plasmid. The following PCR primers were used to screen for correct insertion: 5′-AAAAGGGGGCTGTCCAGTGTGT-3′ (SEQ ID NO: 81, FWD-SOX9scrn-Ex3-2330, outside SOX9 donor region) with 5′-AGCCCTCCATGTGCACCTTGAA-3′ (SEQ ID NO: 82, REV-mCherryscrn-1045, inside SOX9 donor region); 5′-GAAATTGCATCGCATTGTCTGAGTAGG-3′ (SEQ ID NO: 83, FWD BGHpA, inside MIXL1 donor region) and 5′-5537 TTGCATAGCTGTCCTGCAGG-3′ (SEQ ID NO: 84, 3Rev-MX-RHA-scrn, outside MIXL1 donor region).

hiPSC Differentiation

Sclerotome Differentiation. hiPSCs were passaged at 1:12-1:20 as evenly-distributed small aggregates onto Vitronectin-coated 6-well plates. After one day, Essential 8 medium was replaced with CDM2 medium (Loh et al., supra, 2016)—which consisted of 50% IMDM medium (ThermoFisher, Cat. No. 31980030), 50% F12 medium (ThermoFisher, Cat. No. 31765035), 1 mg/mL pre-dissolved polyvinyl alcohol (Sigma, Cat. No. P8136), 1% lipid concentrate (ThermoFisher, Cat. No. 11905031), 450 μM monothioglycerol (Sigma, Cat. No. M6145), 1% insulin-transferrin-selenium, and 1% penicillin/streptomycin. Differentiation to sclerotome was achieved using selective pathway activators and inhibitors supplemented in CDM2 medium, according to Loh et al. supra, (2016). At day 1, hiPSC cultures were treated with 30 ng/mL Activin A (R&D systems, Cat. No. 338-AC-050/CF), 4 μM CHIR99021 (R&D Systems, Cat. No. 4423), 20 ng/mL FGF2 (R&D Systems, Cat. No. 233-FB), and 100 nM PIK90 (Millipore Sigma, Cat. No. 528117) for 24 hours to induce anterior primitive streak. At day 2, cultures were treated with 4 μM SB-431542 (Millipore Sigma, Cat. No. S4317), 3 μM CHIR99021, 250 nM LDN-193189 (Sigma, Cat. No. SML0559), and 20 ng/mL FGF2 for 24 hours to induce paraxial mesoderm. At day 3, cultures were treated with 4 μM SB-431542, 250 nM LDN-193189, 1 μM C59 (Cellagen Technology, Cat. No. C7641-2s), and 500 nM PD173074 (R&D Systems, Cat. No. 3044) for 24 hours to induce early somite/somitomere. At days 4-6, cultures were treated with 2 μM purmorphamine (ReproCell, Cat. No. 04-009) and 1 μM C59 for 72 hours to induce sclerotome; purmorphamine was shown to be an effective substitute for the commercially-synthesized Hedgehog agonist 21K, according to Wu et al. (Nature Communications, 12(1). doi.org/10.1038/s41467-020-20598-y, 2021) Each day, cultures were washed with CDM2 medium prior to adding fresh differentiation medium.

Limb Mesoderm Differentiation. Differentiation of hiPSCs to limb mesoderm was also achieved using selective pathway activators and inhibitors supplemented in CDM2 medium (Loh et al., Cell, 166(2), 451-467, doi.org/10.1016/j.cell.2016.06.011. 2016). At day 1, hiPSC cultures were treated with 30 ng/mL Activin A (R&D systems, Cat. No. 338-AC-050/CF), 6 μM CHIR99021 (R&D Systems, Cat. No. 4423), 20 ng/mL PGF2 (R&D Systems, Cat. No. 233-FB), 100 nM PIK90 (Millipore Sigma, Cat. No. 528117), and 40 ng/mL BMP-4 (R&D systems, Cat. No. 314-BP-050) for 24 hours to induce mid primitive streak. At day 2, cultures were treated with 4 μM SB-431542 (Millipore Sigma, Cat. No. S4317), 30 ng/mL BMP-4, and 1 μM C59 (Cellagen Technology, Cat. No. C7641-2s) for 48 hours to induce lateral plate mesoderm. At day 4, cultures were treated with 4 μM SB-431542, 40 ng/mL BMP-4, 3 μM CHIR99021, 500 nM PD173074 (R&D Systems, Cat. No. 3044) for 24 hours to induce limb mesoderm. Each day, cultures were washed with CDM2 medium prior to adding fresh differentiation medium.

Chondrogenic Differentiation. At day 7, three chondrogenic differentiation strategies were employed from sclerotome cultures derived from NCRM NL5 hiPSC and SOX9-mCherry hiPSC cell lines. The first two strategies involved cell detachment using Accutase, and the pelleting of 5×10⁵ sclerotome cells at 193×g in 1 mL chondrogenic medium—consisting of high glucose Dulbecco's Modified Eagle Medium (DMEM) with 1 mM sodium pyruvate, 1% insulin-transferrin-selenium, 100 nM dexamethasone, 50 ug/ml L-ascorbic acid phosphate magnesium salt n-hydrate, 1% penicillin/streptomycin—in 14 mL conical tubes. In the first approach, pellet cultures were supplemented with 10 ng/mL TGFβ1, and in the second approach cultures were treated with 10 ng/mL TGFβ1, 10 ng/mL BMP-2 (Peprotech, Cat. No. AF-120), and 10 ng/mL GDF-5 (Peprotech, Cat. No. 120-01); in both cases, medium was changed 2-3×/week. The final approach, which is referred to as the formation of chondrospheroids, involved treatment of monolayer sclerotome cultures with chondrogenic medium supplemented with 10 ng/mL TGFβ1, 10 ng/mL BMP-2, and 10 ng/mL GDF-5, with daily medium replacements. After 5-15 days of chondrogenic treatment, developing nodules were loosely adhered or completely detached from monolayer cultures; these chondrospheroids were either gently detached or collected via pipetting and transferred to 14 mL conical tubes (suspension culture) with 1 mL chondrogenic medium supplemented with 1Ong/mL TGFβ1, 1Ong/mL BMP-2, and 10 ng/mL GDF-5, with medium replacements 2-3×/week. If cultures delaminated before day 15, the chondrospheroids were gently removed by gentle pipetting and transferred to suspension culture for continued chondrogenic treatment, as noted above. Tissues were collected at several timepoints and processed for histology, sequencing, and transplantation, as noted above.

Flow Cytometry and FACS: For knockdown studies, hBMSCs/SSCs were treated with collagenase II for 10 minutes at 37° C., then detached using Trypsin/EDTA and washed in FACS buffer-PBS supplemented with 2% FBS, 2 mM EDTA, and 1% penicillin/streptomycin. Live, transduced hBMSCs/SSCs (GFP⁺7AAD⁻) were FACS-sorted using a Sony SH800. For MIXL1-GFP measurements, day 1 anterior primitive streak cells were detached using Accutase and resuspended in PBS supplemented with 0.2% BSA. Cells were acquired using BD LSR Fortessa1 with analysis using FlowJo (Tree Star).

Quantification and Statistical Analysis: Area measurements from maximum intensity projections were performed according to Gadomski et al. (Cell Stem Cell, 29(4), 528-544.e9, doi.org/10.1016/j.stem.2022.02.008, 2022). Following confocal acquisition, the channel of interest was isolated using “Split Channel” and quantified using “Color Threshold” in Fiji/Image J Software (National Institutes of Health). Measurements of positive staining area and total tissue area were obtained, and positive staining area was calculated as a percentage of total tissue area, which did not include HyA-FMB particles. Data are shown as mean±standard error of the mean (SEM) and are representative of at least two independent experiments, unless otherwise noted, with N values representing biological or technical replicates, which are outlined in the figure legends. Unpaired two-tailed t tests were used for two-group comparisons with α=0.05. Significant differences between groups were indicated as: *p<0.05, **p<0.01, ***p<0.001. Statistical analyses and graphics were performed with Microsoft Excel and GraphPad Prism 8 software.

In view of the many possible aspects to which the principles of our invention may be applied, it should be recognized that illustrated aspects are only examples of the invention and should not be considered a limitation on the scope of the invention. Rather, the scope of the invention is defined by the following claims. We therefore claim as our invention all that comes within the scope and spirit of these claims.

We claim:

1. An isolated non-natural human chondrocyte that has decreased expression of Type X Collagen (COL10A1) as compared with hypertrophic adult chondrocytes, and wherein the non-natural human chondrocyte is derived from an induced pluripotent stem cell (iPSC).

2. The isolated non-natural human chondrocyte of claim 1, wherein
a) expression of at least one of secondary ossification center associated regulator of chondrocyte maturation (SNORC), leucine zipper protein (LUZP)2, and stathmin (STMN)2 is increased as compared to adult and fetal human chondrocytes; and/or
b) expression of at least one of CD5, CD27, CD53, tumor necrosis factor superfamily member (TNFRSF)1B, and myogenin (MYOG) is decreased as compared to adult and fetal human chondrocytes.

3. The isolated non-natural human chondrocyte of claim 1 or claim 2, wherein the non-natural human chondrocyte does not undergo hypertrophy for at least 42 days in vitro.

4. The isolated non-natural human chondrocyte of any one of claims 1-3, wherein the non-natural human chondrocyte does not undergo hypertrophy for at least 5 months when transplanted in vivo.

5. A chondrospheroid comprising the non-natural human chondrocyte of any one of claims 1-4.

6. A conjugate comprising the isolated non-natural human chondrocyte of any one of claims 1-5, linked to a solid carrier.

7. The conjugate of claim 6, wherein the solid carrier comprises fibrin.

8. The conjugate of claim 7, wherein the solid carrier is a fibrin microbead.

9. A method of producing the non-natural human chondrocyte of any one of claims 1-4, comprising:
differentiating a chondrospheroid into chondrocytes using an effective amount of TGF, a BMP, and/or a BMPR1A/B agonist, thereby producing chondrocytes.

10. The method of claim 9, further comprising
treating sclerotome cells with an effective amount of a transforming growth factor (TGF)β, a bone morphogenic protein (BMP), and/or a bone morphogenic protein receptor (BMPR)1A/B agonist, thereby producing the chondrospheroid.

11. The method of claim 9 or claim 10, wherein the TGFβ is TGFβ1 or TGFβ3.

12. The method of any one of claims 9-11, and wherein the BMP is BMP-2 or BMP-4.

13. The method of any one of claims 9-12, wherein the BMPR1A/B agonist is growth differentiation factor (GDF)-5, BMP-4, BMP-5, BMP-6, BMP-7, BMP-8A, BMP-8B, BMP-10, BMP-11, GDF-6, GDF-7, or anti-Müllerian hormone (AMH).

14. The method of any one of claims 9-13, wherein the TGFβ is TGFβ1, the BMP is BMP-2, and the BMPR1A/B agonist is GDF-5.

15. The method of claim 14, comprising differentiating the sclerotome cells using a chondrogenic medium comprising 10 ng/mL TGFβ1, 10 ng/mL BMP-2, and 10 ng/mL GDF-5.

16. The method of claim 15, wherein the method comprises:
a) differentiating human induced pluripotent stem cells into anterior primitive streak cells;
b) differentiating the anterior primitive streak cells to paraxial mesoderm cells;
c) differentiating the paraxial mesoderm cells into early somite cells; and
d) differentiating the early somite cells into the sclerotome cells.

17. The method of claim 16, comprising:
a) differentiating the human induced pluripotent stem cells into the anterior primitive streak cells comprises treating the human induced pluripotent cells with an effective amount of Activin A, an effective amount of PI3K inhibitor, a Wnt activator, and FGF2;
b) differentiating the anterior primitive streak cells into the paraxial mesoderm cells with an effective amount of a TGFβ inhibitor, a BMP inhibitor, a Wnt activator and FGF2;
c) differentiating the paraxial mesoderm cells into the early somite cells using an effective amount of a TGFβ inhibitor, a BMP inhibitor, a Wnt inhibitor, and a FGF/ERK inhibitor; and/or
d) differentiating the early somite cells into the sclerotome cells using an effective amount of a Wnt inhibitor and Hedgehog activator.

18. The method of any one of claims 9-17 further comprising digesting the chondrospheroid into single cells.

19. The method of claim 18, wherein digesting the chondrospheroid comprises treating the chondrospheroid with an effective amount of an enzyme to produce non-natural human chondrocytes, wherein expression of: a) at least one of SNORC, LUZP2, and STMN2 is increased in the non-natural human chondrocytes as compared to adult and fetal human chondrocytes; and/or b) at least one of CD5, CD27, CD53, TNFRSF1B, and MYOG is decreased in the non-natural human chondrocytes as compared to adult and fetal human chondrocytes.

20. A method for promoting cartilage growth and/or repair, comprising
administering locally to a site in a subject in need thereof, a therapeutically effective amount of the conjugate of any one of claims 6-8,
thereby producing stable cartilage locally at the site in the subject, wherein the subject has, or is at risk of having, cartilage damage or degradation at the site.

21. The method of claim 20, wherein the cartilage is stable for more than 5 months in the subject.

22. The method of claim 20 or 21, wherein a) expression of at least one of SNORC, LUZP2, and STMN2 is increased in the non-natural human chondrocytes as compared to adult and fetal human chondrocytes; and/or b) expression of at least one of CD5, CD27, CD53, TNFRSF1B, and MYOG is decreased in the non-natural human chondrocytes as compared to adult and fetal human chondrocytes.

23. The method of any one of claims 20-22, wherein administration is via injection to the site.

24. The method of claim 23, wherein the injection is intra-articular or a trans-osseous.

25. The method of any one of claims 20-24, wherein administration is via minimally invasive surgical procedure.

26. The method of any one of claims 19-24, wherein the administration is to a joint.

27. The method of claim 26, wherein the joint is a knee, shoulder, wrist or hip joint.

28. The method of any one of claim 20-27, wherein the subject is at risk of developing, or has, osteoarthritis, cartilage defect, osteochondritis dissecans, osteochondrodysplasia, or cartilage injury.

29. The method of any one of claims 19-26, wherein the subject has bone damage.


SEQUENCE LISTING (84 sequences)

SEQ ID NO: 1 - DNA, 17 bp, Synthetic construct
  source 1..17: mol_type = other DNA
ggagtggacgaggcaag

SEQ ID NO: 2 - DNA, 21 bp, Synthetic construct
  source 1..21: mol_type = other DNA
agcttctgtctgtgccttctg

SEQ ID NO: 3 - DNA, 21 bp, Synthetic construct
  source 1..21: mol_type = other DNA
aacgatttccagttcagggca

SEQ ID NO: 4 - DNA, 20 bp, Synthetic construct
  source 1..20: mol_type = other DNA
agcccagtgttgtagcagaa

SEQ ID NO: 5 - DNA, 18 bp, Synthetic construct
  source 1..18: mol_type = other DNA
cagccgcttcacctacag

SEQ ID NO: 6 - DNA, 21 bp, Synthetic construct
  source 1..21: mol_type = other DNA
ttttgtattcaatcactgtct

SEQ ID NO: 7 - DNA, 17 bp, Synthetic construct
  source 1..17: mol_type = other DNA
actcccagcacgcagaa

SEQ ID NO: 8 - DNA, 21 bp, Synthetic construct
  source 1..21: mol_type = other DNA
tgggccttttatgcctgtggg

SEQ ID NO: 9 - DNA, 19 bp, Synthetic construct
  source 1..19: mol_type = other DNA
actcctcctccggcatgag

SEQ ID NO: 10 - DNA, 18 bp, Synthetic construct
  source 1..18: mol_type = other DNA
gctgcacgtcggttttgg

SEQ ID NO: 11 - DNA, 17 bp, Synthetic construct
  source 1..17: mol_type = other DNA
tcgaggacagcgaggcc

SEQ ID NO: 12 - DNA, 22 bp, Synthetic construct
  source 1..22: mol_type = other DNA
tcgagggtgtagcgtgtagaga

SEQ ID NO: 13 - DNA, 17 bp, Synthetic construct
  source 1..17: mol_type = other DNA
ggcaatagcaggttcac

SEQ ID NO: 14 - DNA, 21 bp, Synthetic construct
  source 1..21: mol_type = other DNA
cgataacagtcttgccccact

SEQ ID NO: 15 - DNA, 17 bp, Synthetic construct
  source 1..17: mol_type = other DNA
agtgagaggcaacctgg

SEQ ID NO: 16 - DNA, 20 bp, Synthetic construct
  source 1..20: mol_type = other DNA
acactcggaccacatccttc

SEQ ID NO: 17 - DNA, 18 bp, Synthetic construct
  source 1..18: mol_type = other DNA
ggtaccccgacatccact

SEQ ID NO: 18 - DNA, 20 bp, Synthetic construct
  source 1..20: mol_type = other DNA
taatctccggcctagccaaa

SEQ ID NO: 19 - DNA, 18 bp, Synthetic construct
  source 1..18: mol_type = other DNA
tgcttccctgagacccag

SEQ ID NO: 20 - DNA, 25 bp, Synthetic construct
  source 1..25: mol_type = other DNA
gatcacttctttcctttgcatcaag

SEQ ID NO: 21 - DNA, 17 bp, Synthetic construct
  source 1..17: mol_type = other DNA
gaggagaaagtggaggt

SEQ ID NO: 22 - DNA, 21 bp, Synthetic construct
  source 1..21: mol_type = other DNA
ctctgatgaggaccgcttctg

SEQ ID NO: 23 - DNA, 17 bp, Synthetic construct
  source 1..17: mol_type = other DNA
caacataaacggactca

SEQ ID NO: 24 - DNA, 20 bp, Synthetic construct
  source 1..20: mol_type = other DNA
accacctctacgaacacatt

SEQ ID NO: 25 - DNA, 17 bp, Synthetic construct
  source 1..17: mol_type = other DNA
gggagcggtgaagatgg

SEQ ID NO: 26 - DNA, 20 bp, Synthetic construct
  source 1..20: mol_type = other DNA
tcatgttgctcacggaggag

SEQ ID NO: 27 - DNA, 18 bp, Synthetic construct
  source 1..18: mol_type = other DNA
gaagtggttccttggcag

SEQ ID NO: 28 - DNA, 20 bp, Synthetic construct
  source 1..20: mol_type = other DNA
tcctgcttgcctcaaagtgt

SEQ ID NO: 29 - DNA, 18 bp, Synthetic construct
  source 1..18: mol_type = other DNA
agcttgggtgcctcctta

SEQ ID NO: 30 - DNA, 20 bp, Synthetic construct
  source 1..20: mol_type = other DNA
tgcttccctgaaagacatca

SEQ ID NO: 31 - DNA, 18 bp, Synthetic construct
  source 1..18: mol_type = other DNA
agcagccgtatctgcacc

SEQ ID NO: 32 - DNA, 21 bp, Synthetic construct
  source 1..21: mol_type = other DNA
ctcctttcggtcacacatgct

SEQ ID NO: 33 - DNA, 17 bp, Synthetic construct
  source 1..17: mol_type = other DNA
aagtaccaaccccgcat

SEQ ID NO: 34 - DNA, 20 bp, Synthetic construct
  source 1..20: mol_type = other DNA
taggctgtcacggagatgaa

SEQ ID NO: 35 - DNA, 17 bp, Synthetic construct
  source 1..17: mol_type = other DNA
gggctctctgagaggca

SEQ ID NO: 36 - DNA, 19 bp, Synthetic construct
  source 1..19: mol_type = other DNA
cctttgctctgcggttctg

SEQ ID NO: 37 - DNA, 18 bp, Synthetic construct
  source 1..18: mol_type = other DNA
cggaattacctgccacct

SEQ ID NO: 38 - DNA, 20 bp, Synthetic construct
  source 1..20: mol_type = other DNA
ggtctgtgagttccccgatg

SEQ ID NO: 39 - DNA, 18 bp, Synthetic construct
  source 1..18: mol_type = other DNA
actccgcgttcagcaacc

SEQ ID NO: 40 - DNA, 21 bp, Synthetic construct
  source 1..21: mol_type = other DNA
tgggttttctgttgcgaggtc

SEQ ID NO: 41 - DNA, 18 bp, Synthetic construct
  source 1..18: mol_type = other DNA
cctcctggtatctcaacc

SEQ ID NO: 42 - DNA, 20 bp, Synthetic construct
  source 1..20: mol_type = other DNA
gagggtcgagttctcaatcc

SEQ ID NO: 43 - DNA, 17 bp, Synthetic construct
  source 1..17: mol_type = other DNA
gagctgaggagagtccc

SEQ ID NO: 44 - DNA, 20 bp, Synthetic construct
  source 1..20: mol_type = other DNA
tgtgcctctctctaggtcca

SEQ ID NO: 45 - DNA, 17 bp, Synthetic construct
  source 1..17: mol_type = other DNA
tctgagcgccaggtcaa

SEQ ID NO: 46 - DNA, 20 bp, Synthetic construct
  source 1..20: mol_type = other DNA
ctgaacttggagaggctgtg

SEQ ID NO: 47 - protein, 18 aa, Synthetic construct
GATTTCAGGCCTGCTGGG

SEQ ID NO: 48 - DNA, 20 bp, Synthetic construct
  source 1..20: mol_type = other DNA
tttcgcaccccttggttaca

SEQ ID NO: 49 - DNA, 17 bp, Synthetic construct
  source 1..17: mol_type = other DNA
ccagctcaggtgacaac

SEQ ID NO: 50 - DNA, 20 bp, Synthetic construct
  source 1..20: mol_type = other DNA
cttggccccttgtgcttttc

SEQ ID NO: 51 - DNA, 18 bp, Synthetic construct
  source 1..18: mol_type = other DNA
ctccacgctccggatagt

SEQ ID NO: 52 - DNA, 20 bp, Synthetic construct
  source 1..20: mol_type = other DNA
atcttgtggcggatgtggtt

SEQ ID NO: 53 - DNA, 18 bp, Synthetic construct
  source 1..18: mol_type = other DNA
tggttatgttgctggaca

SEQ ID NO: 54 - DNA, 20 bp, Synthetic construct
  source 1..20: mol_type = other DNA
ggaagccgtgacagaatgac

SEQ ID NO: 55 - DNA, 18 bp, Synthetic construct
  source 1..18: mol_type = other DNA
gtgcgtcctttaatcctc

SEQ ID NO: 56 - DNA, 19 bp, Synthetic construct
  source 1..19: mol_type = other DNA
gtgagagcaagcggaaaag

SEQ ID NO: 57 - DNA, 18 bp, Synthetic construct
  source 1..18: mol_type = other DNA
agattatatcaggttgta

SEQ ID NO: 58 - DNA, 20 bp, Synthetic construct
  source 1..20: mol_type = other DNA
acacagcggaaacactcgat

SEQ ID NO: 59 - DNA, 18 bp, Synthetic construct
  source 1..18: mol_type = other DNA
taccaccacacccatcaa

SEQ ID NO: 60 - DNA, 20 bp, Synthetic construct
  source 1..20: mol_type = other DNA
acaccaagacagggacagac

SEQ ID NO: 61 - DNA, 18 bp, Synthetic construct
  source 1..18: mol_type = other DNA
tgatgcttttgtgcgaga

SEQ ID NO: 62 - DNA, 21 bp, Synthetic construct
  source 1..21: mol_type = other DNA
agggaagcgtttttattggct

SEQ ID NO: 63 - DNA, 18 bp, Synthetic construct
  source 1..18: mol_type = other DNA
aactccttctcggggcgt

SEQ ID NO: 64 - DNA, 21 bp, Synthetic construct
  source 1..21: mol_type = other DNA
catcccattgtaattgtagcc

SEQ ID NO: 65 - DNA, 17 bp, Synthetic construct
  source 1..17: mol_type = other DNA
ggcgacggagaacacaa

SEQ ID NO: 66 - DNA, 19 bp, Synthetic construct
  source 1..19: mol_type = other DNA
ctgggcacaggacgacttc

SEQ ID NO: 67 - DNA, 18 bp, Synthetic construct
  source 1..18: mol_type = other DNA
caagtgtgcgtctgcctt

SEQ ID NO: 68 - DNA, 21 bp, Synthetic construct
  source 1..21: mol_type = other DNA
cagctctttcttttcggctct

SEQ ID NO: 69 - DNA, 17 bp, Synthetic construct
  source 1..17: mol_type = other DNA
atggggaaggtgaaggt

SEQ ID NO: 70 - DNA, 20 bp, Synthetic construct
  source 1..20: mol_type = other DNA
taaaagcagccctggtgacc

SEQ ID NO: 71 - DNA, 60 bp, Synthetic construct
  source 1..60: mol_type = other DNA
agtggattctgggagaattcgagataagctctgagaagccatgactgacagcctgagaga

SEQ ID NO: 72 - DNA, 25 bp, Synthetic construct
  source 1..25: mol_type = other DNA
caccgtgaggattctgggagaattc

SEQ ID NO: 73 - DNA, 25 bp, Synthetic construct
  source 1..25: mol_type = other DNA
aaacgaattctcccagaatcctcac

SEQ ID NO: 74 - DNA, 25 bp, Synthetic construct
  source 1..25: mol_type = other DNA
caccgagaattcgggataagctctg

SEQ ID NO: 75 - DNA, 25 bp, Synthetic construct
  source 1..25: mol_type = other DNA
aaaccagagcttatcccgaattctc

SEQ ID NO: 76 - DNA, 34 bp, Synthetic construct
  source 1..34: mol_type = other DNA
tatacgaagttatactagtggaggcctcccacga

SEQ ID NO: 77 - DNA, 25 bp, Synthetic construct
  source 1..25: mol_type = other DNA
caccgcagctcactcgaccttgagg

SEQ ID NO: 78 - DNA, 25 bp, Synthetic construct
  source 1..25: mol_type = other DNA
aaaccctcaaggtcgagtgagctgc

SEQ ID NO: 79 - DNA, 25 bp, Synthetic construct
  source 1..25: mol_type = other DNA
caccgcttgaggaggcctcccacga

SEQ ID NO: 80 - DNA, 25 bp, Synthetic construct
  source 1..25: mol_type = other DNA
aaactcgtgggaggcctcctcaagc

SEQ ID NO: 81 - DNA, 22 bp, Synthetic construct
  source 1..22: mol_type = other DNA
aaaagggggctgtccagtgtgt

SEQ ID NO: 82 - DNA, 22 bp, Synthetic construct
  source 1..22: mol_type = other DNA
agccctccatgtgcaccttgaa

SEQ ID NO: 83 - DNA, 27 bp, Synthetic construct
  source 1..27: mol_type = other DNA
gaaattgcatcgcattgtctgagtagg

SEQ ID NO: 84 - DNA, 20 bp, Synthetic construct
  source 1..20: mol_type = other DNA
ttgcatagctgtcctgcagg